(19) United States
(12) Patent Application Publication
(10) Pub. No.: US 2006/0148721 A1
(43) Pub. Date: Jul. 6, 2006

(54) Combination therapy for the treatment of dyslipidemia

(76) Inventor: Ngozi E. Erondu, Englishtown, NJ (US)

Correspondence Address: MERCK AND CO., INC, P O BOX 2000, RAHWAY, NJ, 07065-0907, US

(21) Appl. No.: 10/555,194

(22) PCT Filed: Jun. 2, 2004
(86) PCT No.: PCT/US04/17120

§ 371 Date: Nov. 1, 2005

Related U.S. Application Data
(60) Provisional application No. 60/476,387, filed on Jun. 6, 2003.

(51) Int. Cl. A61K 31/506; A61K 31/497; A61K 31/7024; A61K 31/4747; A61K 31/353

(52) U.S. Cl. 514/23; 514/255.05; 514/278; 514/256; 514/454

(57) ABSTRACT

The present invention relates to compositions comprising an anti-obesity agent and an anti-dyslipidemic agent useful for the treatment of dyslipidemia, dyslipidemia associated with obesity and dyslipidemia-related disorders. The present invention further relates to methods of treating or preventing obesity, and obesity-related disorders, in a subject in need thereof by administering a composition of the present invention. The present invention further provides for pharmaceutical compositions, medicaments, and kits useful in carrying out these methods.

### BACKGROUND OF THE INVENTION

Disorders of lipid metabolism, or dyslipidemias, include various conditions characterized by abnormal concentrations of one or more lipids (i.e. cholesterol and triglycerides), and/or apolipoproteins (i.e., apolipoproteins A, B, C and E), and/or lipoproteins (i.e., the macromolecular complexes formed by the lipid and the apolipoprotein that allow lipids to circulate in blood, such as LDL, VLDL and IDL). Hyperlipidemia is associated with abnormally high levels of lipids, LDL and VLDL cholesterol, and/or triglycerides. Cholesterol is mostly carried in Low Density Lipoproteins (LDL), and this component is commonly known as the “bad” cholesterol because it has been shown that elevations in LDL-cholesterol correlate closely to the risk of coronary heart disease. A smaller component of cholesterol is carried in the High Density Lipoproteins and is commonly known as the “good” cholesterol. In fact, it is known that the primary function of HDL is to accept cholesterol deposited in the arterial wall and to transport it back to the liver for disposal through the intestine. Although it is desirable to lower elevated levels of LDL cholesterol, it is also desirable to increase levels of HDL cholesterol. Generally, it has been found that increased levels of HDL are associated with lower risk for coronary heart disease (CHD). See, for example, Gordon, et al., Am. J. Med., 62, 707-714 (1977); Stampfer, et al., N. England J. Med., 325, 373-381 (1991); and Kannel, et al., Ann. Internal Med., 90, 85-91 (1979). An example of an HDL raising agent is nicotinic acid, a drug with limited utility because doses that achieve HDL raising are associated with undesirable effects, such as flushing.

Dyslipidemias were originally classified by Fredrickson according to the combination of alterations mentioned above. The Fredrickson classification includes 6 phenotypes (i.e., I, IIa, IIb, III, IV and V) with the most common being the isolated hypercholesterolemia (or type IIa) which is usually accompained by elevated concentrations of total and LDL cholesterol. The initial treatment for hypercholesterolemia is often aimed at avoiding risk factors such as obesity, and at modifying the diet to one low in fat and cholesterol, coupled with appropriate physical exercise, followed by drug therapy when LDL-lowering goals are not met by diet and exercise alone.

A second common form of dyslipidemia is the mixed or combined hyperlipidemia or type IIb and III of the Fredrickson classification. This dyslipidemia is often prevalent in patients with type 2 diabetes, obesity and metabolic syndrome. In this dyslipidemia there are modest elevations of LDL-cholesterol, accompanied by more pronounced elevations of small dense LDL-cholesterol particles, VLDL and/or IDL (i.e., triglyceride rich lipoproteins), and total triglycerides. In addition, concentrations of HDL are often low.

The risk of atherosclerosis and coronary artery or carotid artery disease, and therefore the risk of having a heart attack or stroke, increases as the total cholesterol level increases. Atherosclerosis refers to a disease in which the wall of an artery becomes thicker and less elastic. In atherosclerosis, fatty material accumulates under the inner lining of the arterial wall. Atherosclerosis can affect the arteries of the brain, heart, kidneys, other vital organs, and the arms and legs. When atherosclerosis develops in the arteries that supply the brain (carotid arteries), a stroke may occur; when it develops in the arteries that supply the heart (coronary arteries), a heart attack may occur. Arteries affected with atherosclerosis lose their elasticity, an as the atheromas (patchy thickening in the inner lining of the artery) grow, the arteries narrow. With time, the atheromas collect calcium deposits, may become brittle, and may rupture. Blood may then enter a ruptured atheroma, making it larger, so that it narrows the artery even more. A ruptured atheroma also may spill its fatty contents and trigger the formation of a blood clot (thrombus). The clot may further narrow or even occlude the artery, or it may detach and float downstream where it causes an occlusion (embolism).

Causes of high cholesterol levels include a diet high in saturated fats and cholesterol; cirrhosis, poorly controlled diabetes, underactive thyroid gland, overactive pituitary gland, kidney failure, porphyria, and heredity. Causes of high triglyceride levels include excess calories in diet, severe uncontrolled diabetes, kidney failure, acute alcohol abuse, certain drugs, and heredity.

Obesity is one of the factors contributing to high levels of certain lipids, such as VLDL and LDL, as a result, the initial treatment for overweight patients with high cholesterol or triglyceride levels is to lose weight. Levels of fatty material such as cholesterol and triglycerides may also be controlled with a variety of drugs. Hydroxymethylglutaryl-coenzyme A (HMG-CoA) reductase inhibitors block the synthesis of cholesterol and enhance the removal of low density lipoproteins from the bloodstream. HMG-CoA synthase inhibitors inhibit the biosynthesis of hydroxymethylglutaryl-coenzyme A from acetyl-coenzyme A. Lipoprotein synthesis inhibitors reduce the rate of very low density lipoprotein production. Bile acid sequestrants, or absorbers, bind bile acids in the intestine and enhance low density lipoprotein removal from the bloodstream. Renin angiotensin system inhibitors may also decrease the formation of atherosclerotic plaques. Cholesterol absorption inhibitors inhibit intestinal cholesterol absorption by blocking the movement of cholesterol from the intestinal lumen into enterocytes of the small intestinal wall. Cholesteryl ester transfer protein (CETP) inhibitors inhibit the CETP mediated transport of various cholesteryl esters and triglycerides from HDL to LDL and VLDL. Other compounds reduce serum cholesterol levels primarily by mechanisms of action such as squalene synthetase inhibition, acyl coenzyme A—cholesterol acyl transferase (ACAT) inhibition, triglyceride synthesis inhibition, MTP inhibition, bile acid sequestration, and transcription modulation, such as agonists or antagonists of nuclear hormones.

Additionally, three sub-types of peroxisome proliferator activated receptor (PPAR) have been discovered and described; they are peroxisome proliferator activated receptor alpha (PPARα), peroxisome proliferator activated receptor gamma (PPARγ), and peroxisome proliferator activated receptor delta (PPARδ). Fibric acid derivatives such as clofibrate, fenofibrate, benzafibrate, ciprofibrate, beclofibrate and etofibrate, as well as gemfibrozil, each of which are PPARα ligands and/or activators, produce a substantial reduction in plasma triglycerides as well as some increase in HDL. The effects on LDL cholesterol are inconsistent and might depend upon the compound and/or the dyslipidemic phenotype. For these reasons, this class of compounds has been primarily used to treat hypertriglyceridemia (i.e, Fredrickson Type IV and V) and/or mixed hyperlipideniua.

It has been disclosed in WO97/28149 that agonists of PPARδ are useful in raising HDL plasma levels. WO97/27857, 97/28115, 97/28137 and 97/27847 disclose compounds that are useful as antidiabetic, antiobesity, anti-atherosclerosis and antihyperlipidemic agents, and which may exert their effect through activation of PPARs. PPARα agonists should improve the lipid profile and alleviate dyslipidemias by reducing elevated LDL levels and elevated triglyceride levels and/or increasing HDL levels.

Left ventricular hypertrophy (LVH), which is characterized by thickening of the left ventricular wall, is due to the response of the heart to chronic pressure or volume overload. Left ventricular hypertrophy is defined as a left ventricular mass index exceeding 131 g/m² of the body surface area in men, and 100 g/m² in women (Savage et al., The Framingham Study, Circulation, 75 (1 Pt 2): 26-33 (1987).

Left ventricular hypertrophy is independently associated with increased incidence of cardiovascular disease, such as congestive heart failure, ischaemic heart disease, cardiovascular and all-cause mortality, sudden death, and stroke. Regression of left ventricular hypertrophy has been associated with a reduction in cardiovascular risk. It has also been found that the incidence of morbid events in patients with progression of left ventricular hypertrophy is greater than in patients with regression of left ventricular hypertrophy.

Dyslipidemia and a serum cholesterol ester fatty acid composition indicating a high dietary intake of saturated and monounsaturated fats, as well as, obesity and hypertension, at age 50 have been shown to be predictive of the prevalence of LVH at age 70 for men (Sundstroem, J. et al., Circulation, February, 836-840 (2001). The impact of obesity, dyslipidemia, and the evidence of high dietary intake of saturated fats on left ventricular hypertrophy was found to be independent of the history of ischemic heart disease, valvular disease, and the use of antihypertensive medication. Associations have also been found between left ventricular hypertrophy and metabolic syndrome (Lind, L. et al., J Hypertens. 13:433-38 (1995).

Metabolic syndrome, also known as syndrome X, is characterized by insulin resistance, along with abdominal obesity, hyperinsulinemia, high blood pressure, low HDL and high VLDL. Although the causal relationship between the various components of metabolic syndrome remains to be confirmed, insulin resistance appears to play an important role (Requen, G. M., et al., N. Eng. J. Med. 334:374-381 (1996); Despres, J-P., et al., N. Engl. J. Med. 334:952-957 (1996); Wajchenberg, B. L., et al., Diabetes/Metabolism Rev. 10: 19-29 (1994)). Metabolic syndrome patients are at increased risk of developing the cardiovascular complications listed above.

Obesity, which can be defined as a body weight more than 20% above the ideal body weight, is a major health concern in Western societies. It is estimated that about 97 million adults in the United States are overweight or obese. Obesity is the result of a positive energy balance, as a consequence of increased ratio of caloric intake to energy expenditure. The molecular factors regulating food intake and body weight balance are incompletely understood. [B. Staels et al., J. Biol. Chem. 270(27), 15958 (1995); F. Lonnquist et al., Nature Medicine 1(9), 950 (1995)]. Although the genetic and/or environmental factors leading to obesity are poorly understood, several genetic factors have been identified.

Epidemiological studies have shown that increasing degrees of overweight and obesity are important predictors of decreased life expectancy. Obesity causes or exacerbates many health problems, both independently and in association with other diseases. The medical problems associated with obesity, which can be serious and life-threatening, include type 2 diabetes mellitus, hypertension, elevated plasma insulin concentrations, insulin resistance, dyslipidemias, hyperlipidemia, metabolic syndrome, endometrial, breast, prostate, kidney, and colon cancer, osteoarthritis, respiratory complications, such as obstructive sleep apnea, gallstones, arterioscelerosis, heart disease, abnormal heart rhythms, and heart arrythmias (Kopelman, P. G., Nature 404, 635-643 (2000)).

Abdominal obesity has been linked with a much higher risk of coronary artery disease, and with three of its major risk factors: high blood pressure, diabetes that starts in adulthood, and high levels of fats (lipids) in the blood. Losing weight dramatically reduces these risks. Abdominal obesity is further closely associated with glucose intolerance, hyperinsulinemia, hypertriglyceridemia, and other disorders associated with metabolic syndrome (syndrome X), such as raised high blood pressure, decreased levels of high density lipoproteins (HDL) and increased levels of very low density lipoproteins (VLDL) (Montague et al., Diabetes, 2000, 49: 883-888).

Obesity is also associated with metabolic syndrome, cardiac hypertrophy, in particular left ventricular hypertrophy, premature death, and with a significant increase in mortality and morbidity from stroke, myocardial infarction, congestive heart failure, coronary heart disease, and sudden death.

Obesity and obesity-related disorders are often treated by encouraging patients to lose weight by reducing their food intake or by increasing their exercise level, thereby increasing their energy output. A sustained weight loss of 5% to 10% of body weight has been shown to improve the co-morbidities associated with obesity and can lead to improvement of obesity-related disorders such as diabetes, left ventricular hypertrophy, osteoarthritis, and pulmonary and cardiac dysfunction.

Weight loss drugs used for the treatment of obesity include orlistat (Davidson, M. H. et al. (1999) JAMA 281:23542), dexfenfluramine (Guy Grand, B. et al. (1989) Lancet 2:1142-5), sibutramine (Bray, G. A. et al. (1999) Obes. Res. &:189-98) and phentermine (Douglas, A. et al. (1983) Int. J. Obes. 7:591-5). However, the side effects of these drugs and anti-obesity agents may limit their use. Dexfenfluramine was withdrawn from the market because of suspected heart valvulopathy; orlistat is limited by gastrointestinal side effects; and the use of sibutramine is limited by its cardiovascular side effects, which have led to reports of deaths and its withdrawal from the market in Italy.

There is a continuing need for new methods of treating dyslipidemia, dyslipidemia associated with obesity and dyslipidemia-related disorders. There is currently no effective treatment for obesity, obesity related disorders, metabolic syndrome, cardiac hypertrophy and left ventricular hypertrophy.

The present invention addresses this problem by providing a combination therapy comprising of at least one anti-obesity agent and at least one anti-dyslipidemic agent for the treatment of dyslipidemia, dyslipidemia associated with obesity, and dyslipidemia-related disorders. The combination of an anti-obesity agent and an anti-dyslipidemic agent, at their respective clinical doses, is expected to be more effective than treatment with either agent alone. Treatment with a combination of an anti-obesity agent and an anti-dyslipidemic agent at sub-clinical doses is expected to produce clinical efficacy with fewer side effects than treatment with either single agent at the monotherapy clinical dose. As a result, combination therapy is more likely to achieve the desired medical benefits without the trial and error involved in prescribing each agent individually during primary care.

The present invention also provides a method for treating, controlling, or preventing obesity and obesity-related disorders, such as cardiac hypertrophy and left ventricular hypertrophy.

The present invention further provides a method for synergistically treating, controlling, or preventing metabolic syndrome comprised of administering the compositions of the present invention in combination with an anti-hypertensive agent and/or an anti-diabetic agent to a subject in need thereof. Metabolic syndrome is a multi-factorial disease characterized by obesity, diabetes, hypertension and dyslipidemia. Due to the polygenic nature of the metabolic syndrome etiology, it is predicted that the combination therapies of the present invention will be more effective than currently available monotherapies in treating or reducing the risk of metabolic syndrome. Combinations of different agents with different modes of action, e.g., a combination of an anti-obesity agent, an anti-dyslipidemic agent, an anti-diabetic agent, and an anti-hypertensive agent, will achieve a better outcome relative to monotherapies using agents with only one mode of action. Additionally, combination therapy is more likely to achieve the desired medical benefits without the trial and error of prescribing each agent alone in primary care.

### SUMMARY OF THE INVENTION

The present invention provides compositions comprising at least one anti-obesity agent and at least one anti-dyslipidemic agent useful in the treatment, control and/or prevention of dyslipidemia, dyslipidemia associated with obesity, and dyslipidemia-related disorders, obesity and obesity-related disorders.

The present invention provides compositions comprising an anti-obesity agent selected from the group consisting of: a 5HT (serotonin) transporter inhibitor, a NE (norepinephrine) transporter inhibitor, a CB-1 (cannabinoind-1 receptor) antagonist/inverse agonist, a ghrelin antibody, a ghrelin antagonist, a H3 (histamine H3) antagonist/inverse agonist, a MCH1R (melanin concentrating hormone 1R) antagonist, a MCH2R (melanin concentrating hormone 2R) agonist/antagonist, a NPY1 (neuropeptide Y Y1) antagonist, a NPY2 (neuropeptide Y Y2) agonist, a NPY5 (neuropeptide Y Y5) antagonist, leptin, a leptin derivative, an opioid antagonist, an orexin antagonist, a BRS3 (bombesin receptor subtype 3) agonist, a CCK-A (cholecystokinin-A) agonist, a CNTF (ciliary neurotrophic factor), a CNTF derivative, a GHS (growth hormone secretagogue receptor) agonist, 5HT2c (serotonin receptor 2c) agonist, a Mc3r (melanocortin 3 receptor) agonist, a Mc4r (melanocortin 4 receptor) agonist, a monoamine reuptake inhibitor, a serotonin reuptake inhibitor, a GLP-1 (glucagon-like peptide 1) agonist, topiramate, phytopharm compound 57, an ACC2 (acetyl-CoA carboxylase-2) inhibitor, a β3 (beta adrenergic receptor 3) agonist, a DGAT1 (diacylglycerol acyltransferase 1) inhibitor, a DGAT2 (diacylglycerol acyltransferase 2)inhibitor, a FAS (fatty acid synthase) inhibitor, a PDE (phosphodiesterase) inhibitor, a thyroid hormone β agonist, an UCP-1 (uncoupling protein 1), 2,.or 3 activator, an acyl-estrogen, a glucocorticoid antagonist, an 11β HSD-1 (11-beta hydroxy steroid dehydrogenase type 1) inhibitor, a SCD-1 (stearoyl-CoA desaturase-1) inhibitor, a dipeptidyl peptidase IV (DP-IV) inhibitor, a lipase inhibitor, a fatty acid transporter inhibitor, a dicarboxylate transporter inhibitor, a glucose transporter inhibitor, a phosphate transporter inhibitor, and pharmaceutically acceptable salts and esters thereof.

The present invention provides compositions comprising an anti-dyslipidemic agent selected from the group consisting of:

(1) a bile acid sequesterant,

(2) a HMG-CoA reductase inhibitor,

(3) a HMG-CoA synthase inhibitor,

(4) a cholesterol absorption inhibitor,

(5) an acyl coenzyme A-cholesterol acyl transferase (ACAT) inhibitor,

(6) a cholesteryl ester transfer protein (CETP) inhibitor,

(7) a squalene synthetase inhibitor,

(8) an anti-oxidant,

(9) a PPAR α agonist,

(10) a FXR receptor modulator,

(11) a LXR receptor agonist,

(12) a lipoprotein synthesis inhibitor,

(13) a renin angiotensin system inhibitor,

(14) a microsomal triglyceride transport inhibitor,

(15) a bile acid reabsorption inhibitor,

(16) a PPARδ agonist,

(17) a triglyceride synthesis inhibitor,

(18) a transcription modulator,

(19) a squalene epoxidase inhibitor,

(20) a low density lipoprotein (LDL) receptor inducer,

(21) a platelet aggregation inhibitor,

(22) a 5-LO or FLAP inhibitor,

(23) a PPARδ partial agonist, and

(24) niacin or a niacin receptor agonist,

and pharmaceutically acceptable salts and esters thereof.

The compositions of the present invention are expected to be efficacious in the treatment of lipid disorders, such as dyslipidemia, hyperlipidemia, hypercholesterolemia, impaired fasting glucose, hyperinsulinemia, hyper-triglyceridemia, low levels of high density lipoprotein cholesterol, high levels of plasma low density lipoprotein cholesterol, high levels of non-high density lipoprotein cholesterol and/or total cholesterol, high levels of plasma triglycerides. The compositions of the present invention are also expected to be efficacious for treating, preventing or reducing the risk of developing atherosclerosis, as well as for halting or slowing the progression of atherosclerotic disease once it has become clinically evident. The compositions of the present invention are also useful in the treatment, control and/or prevention of coronary artery or carotid artery disease, heart attack, and stroke.

The compositions of the present invention are also useful in the treatment, control and/or prevention of obesity, overeating, binge eating, bulimia, elevated plasma insulin concentrations, insulin resistance, glucose tolerance intolerance, lipid disorders, low HDL levels, high LDL levels; hyperglycemia, neoplastic conditions, such as endometrial, breast, prostate, kidney, and colon cancer, osteoarthritis, obstructive sleep apnea, gallstones, abnormal heart rhythms, heart arrythmias, atherosclerosis, myocardial infarction, congestive heart failure, sudden death, impaired fertility, ovarian hyperandrogenism, (polycystic ovary disease), craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia.

Neuropeptide Y (NPY), via G protein-coupled NPY Y5 receptors (NPY5), is implicated in the development of cardiac hypertrophy, and left ventricular hypertrophy, during chronic stimulation of the sympathetic system by potentiating α-adrenergic signals. Recent studies have shown that agonism of the NPY5 receptor in rodent cardiac myocytes may mediate hypertrophy (Bell, D. et al., J-Pharmacol-Exp-Ther. 303: 581-91 (2002)).

NPY5 antagonists are expected to be beneficial in the treatment and/or prevention of cardiac hypertrophy. Furthermore, combination therapy with an anti-dyslipidemic agent and a NPY5 antagonist is beneficial for the treatment of cardiac hypertrophy, particularly left ventricular hypertrophy, associated with dyslipidemia and obesity. The combination of an anti-dyslipidemic agent and a NPY5 antagonist will lower weight and treat, reduce or prevent cardiac hypertrophy leading to improved safety due to reduced cardiac side effects and improved cardiac function in obese dyslipidemic patients. The combination of a NPY5 antagonist and an anti-dyslipidemic agent is expected to treat, reduce and/or prevent the cardiac hypertrophy, in particular the left ventricular hypertrophy, in a subject in need thereof with greater efficacy than either compound alone.

The compositions of the present invention are further useful in the treatment, control and/or prevention of cardiac hypertrophy, particularly left ventricular hypertrophy.

The compositions of the present invention are further useful in the treatment, control and/or prevention of metabolic syndrome.

The present invention is also concerned with treatment of these conditions, and the use of the compositions of the present invention for manufacture of a medicament useful for treating these conditions.

The invention is also concerned with pharmaceutical compositions comprising an anti-obesity agent and an anti-dyslipidemic agent, as active ingredients.

The present invention is also concerned with the use of an anti-obesity agent and an anti-dyslipidemic agent, for the manufacture of a medicament for the treatment of dyslipidemia, dyslipidemia associated with obesity, and dyslipidemia-related disorders, obesity, and obesity-related disorders, which comprises an effective amount of the anti-obesity agent and the anti-dyslipidemic agent, together or separately.

The present invention is also concerned with a product containing an anti-obesity agent and an anti-dyslipidemic agent, as a combined preparation for simultaneous, separate or sequential use in dyslipidemia, dyslipidemia associated with obesity, and dyslipidemia -related disorders, obesity, and obesity-related disorders.

The present invention also relates to the treatment of dyslipidemia, dyslipidemia associated with obesity, dyslipidemia-related disorders, obesity, and obesity-related disorders with a combination of an anti-obesity agent and an anti-dyslipidemic agent, which may be administered separately.

The invention also relates to combining separate pharmaceutical combinations into a kit form.

### DETAILED DESCRIPTION OF THE INVENTION

The present invention provides compositions comprising at least one anti-obesity agent and at least one anti-dyslipidemic agent useful in the treatment or prevention of dyslipidemia, dyslipidemia associated with obesity, dyslipidemia-related disorders, obesity and obesity-related disorders.

The methods and compositions of the present invention comprise an anti-obesity agent. The anti-obesity agent useful in the compositions of the present invention may be any agent useful to decrease food intake known in the art. The anti-obesity agent may be peptidal or non-peptidal in nature, however, the use of a non-peptidal agent is preferred. For convenience, the use of an orally active anti-obesity agent is also preferred.

In one embodiment of the present invention, the anti-obesity agent useful in the compositions of the present invention is selected from the group consisting of:

(1) a 5HT transporter inhibitor,

(2) a NE transporter inhibitor,

(3) a CB-1 antagonist/inverse agonist,

(4) a ghrelin antibody,

(5) a ghrelin antagonist,

(6) a H3 antagonist/inverse agonist,

(7) a MCH1R antagonist,

(8) a MCH2R agonist/antagonist,

(9) a NPY1 antagonist,

(10) a NPY2 agonist,

(11) a NPY5 antagonist,

(12) leptin,

(13) a leptin derivative,

(14) an opioid antagonist,

(15) an orexin antagonist,

(16) a BRS3 agonist,

(17) a CCK-A agonist,

(18) a CNTF,

(19) a CNTF derivative,

(20) a GHS agonist,

(21) 5HT2c agonist,

(22) a Mc3r agonist,

(23) a Mc4r agonist,

(24) a monoamine reuptake inhibitor,

(25) a serotonin reuptake inhibitor,

(26) GLP-1 agonist,

(27) DP-IV inhibitor,

(28) topiramate,

(29) phytopharm compound 57,

(30) an ACC2 inhibitor,

(31) a β3 agonist,

(32) a DGAT1 inhibitor,

(33) a DGAT2 inhibitor,

(34) a FAS inhibitor,

(35) a PDE inhibitor,

(36) a thyroid hormone β agonist,

(37) an UCP-1, 2, or 3 activator,

(38) an acyl-estrogen,

(39) a glucocorticoid antagonist,

(40) an 11β HSD-1 inhibitor,

(41) a SCD-1 inhibitor,

(42) a lipase inhibitor,

(43) a fatty acid transporter inhibitor,

(44) a dicarboxylate transporter inhibitor,

(45) a glucose transporter inhibitor, and

(46) a phosphate transporter inhibitor;

and pharmaceutically acceptable salts and esters thereof. In another embodiment of the present invention, the anti-obesity agent is selected from the group consisting of:

(1) a 5HT transporter inhibitor;

(2) a NE transporter inhibitor;

(3) a CB-1 antagonist/inverse agonist;

(4) a ghrelin antagonist;

(5) a H3 antagonist/inverse agonist;

(6) a MCH1R antagonist;

(7) a MCH2R agonist/antagonist;

(8) a NPY1 antagonist;

(9) a NPY2 agonist;

(10) a NPY5 antagonist;

(11) an opioid antagonist;

(12) an orexin antagonist;

(13) a BRS3 agonist;

(14) a CCK-A agonist;

(15) a CNTF;

(16) a CNTF derivative;

(17) a GHS agonist;

(18) 5HT2c agonist;

(19) a Mc3r agonist;

(20) a Mc4r agonist;

(21) a monoamine reuptake inhibitor;

(22) a serotonin reuptake inhibitor;

(23) GLP-1 agonist;

(24) DP-IV inhibitor;

(25) topiramate;

(26) phytopharm compound 57;

(27) an ACC2 inhibitor;

(28) a β3 agonist;

(29) a DGAT1 inhibitor;

(30) a DGAT2 inhibitor;

(31) a FAS inhibitor;

(32) a PDE inhibitor;

(33) a thyroid hormone β agonist;

(34) an UCP-1, 2, or 3 activator;

(35) an acyl-estrogen;

(36) a glucocorticoid antagonist;

(37) an 11β HSD-1 inhibitor;

(38) a SCD-1 inhibitor;

(39) a lipase inhibitor;

(40) a fatty acid transporter inhibitor;

(41) a dicarboxylate transporter inhibitor; and

(42) a glucose transporter inhibitor;

and pharmaceutically acceptable salts and esters thereof.

In a class of this embodiment, the anti-obesity agent is a CB-1 antagonist/inverse agonist, and pharmaceutically acceptable salts or esters thereof. In a subclass of this class, the CB-1 antagonist/inverse agonist is selected from rimonabant, and pharmaceutically acceptable salts or esters thereof.

In another class of this embodiment, the anti-obesity agent is an opioid antagonist, and pharmaceutically acceptable salts or esters thereof. In a subclass of this class, the opioid antagonist is selected from nalmefene, and pharmaceutically acceptable salts or esters thereof.

In another class of this embodiment, the anti-obesity agent is a CNTF derivative, and pharmaceutically acceptable salts or esters thereof. In subclass of this class, the CNTF derivative is selected from axokine, and pharmaceutically acceptable salts or esters thereof.

In another class of this embodiment, the anti-obesity agent is a monoamine reuptake inhibitor, and pharmaceutically acceptable salts or esters thereof. In a subclass of this class, the monoamine reuptake inhibitor is selected from sibutramine, and pharmaceutically acceptable salts and esters thereof.

In another class of this embodiment, the anti-obesity agent is an acyl-estrogen, and pharmaceutically acceptable salts or esters thereof. In a subclass of this class, the acyl-estrogen, is selected from oleoyl-estrone, and pharmaceutically acceptable salts or esters thereof.

In a class of this embodiment, the anti-obesity agent is a lipase inhibitor, and pharmaceutically acceptable salts or esters thereof, In a subclass of this embodiment, the lipase inhibitor is orlistat, and the pharmaceutically acceptable salts thereof.

In another class of this embodiment, the anti-obesity agent is a NPY2 agonist, and pharmaceutically acceptable salts or esters thereof. In a subclass of this class, the NPY2 agonist is selected from the group consisting of peptide YY (PYY), and PYY₃₋₃₆, and pharmaceutically acceptable salts thereof. In another subclass of this class, the NPY2 agonist is PYY₃₋₃₆, and pharmaceutically acceptable salts thereof.

In another class of this embodiment, the anti-obesity agent is a NPY5 antagonist, and pharmaceutically acceptable salts or esters thereof.

In a subclass of this class, the NPY5 antagonists useful in the present invention are represented by the compounds of structural Formula I: 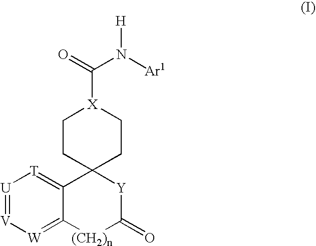
and pharmaceutically acceptable salts and esters thereof, wherein
Ar¹ is selected from the group consisting of:
(1) aryl, and
(2) heteroaryl,
   wherein the aryl and heteroaryl groups are unsubstituted or optionally substituted with a substituent selected from the group consisting of:
   (a) halogen,
   (b) nitro,
   (c) lower alkyl,
   (d) halo(lower)alkyl,
   (e) hydroxy(lower)alkyl,
   (f) cyclo(lower)alkyl,
   (g) lower alkenyl,
   (h) lower alkoxy,
   (i) halo(lower)alkoxy,
   (j) lower alkylthio,
   (k) carboxyl,
   (l) lower alkanoyl,
   (m) lower alkoxycarbonyl,
   (n) lower alkylene optionally substituted with oxo, and
   (o) -Q-Ar²;
      Ar² is selected from the group consisting of
(1) aryl, and
(2) heteroaryl,
   wherein aryl and heteroaryl are unsubstituted or optionally substituted with a substituent selected from the group consisting of:
   (a) halogen,
   (b) cyano,
   (c) lower alkyl,
   (d) halo(lower)alkyl,
   (e) hydroxy(lower)alkyl,
   (f) hydroxy,
   (g) lower alkoxy,
   (h) halo(lower)alkoxy,
   (i) lower alkylamino,
   (j) di-lower alkylamino,
   (k) lower alkanoyl, and
   (l) aryl;
      n is 0 or 1;
      Q is selected from the group consisting of a single bond or carbonyl;
      T, U, V and W are each independently selected from the group consisting of
(1) nitrogen, and
(2) methine,
wherein the methine group is unsubstituted or optionally substituted with a substituent selected from the group consisting of
   (a) halogen,
   (b) lower alkyl,
   (c) hydroxy, and
   (d) lower alkoxy; and
      wherein at least two of T, U, V, and W are methine;
      X is selected from the group consisting of
(1) nitrogen, and
(2) methine; and
   Y is selected from the group consisting of
(1) imino, unsubstituted or optionally substituted with lower alkyl, and
(2) oxygen.

In a sub-class of this subclass, the NPY5 antagonist is selected from the group consisting of:

(1) N-(4-benzoylphenyl)-3-oxospiro[isoindoline-1,4′-piperidine]-1′-carboxamide,

(2) 3-oxo-N-(5-phenyl-2-pyrazinyl)spiro[isoindoline-1,4′-piperidine]-1′-carboxamide,

(3) N-(7-methyl-2-quinolyl)-3-oxospiro[isoindoline-1,4′-piperidine]-1′-carboxamide,

(4) N-(4-benzoylphenyl)-2-methyl-3-oxospiro[isoindoline-1,4′-piperidine]-1′-carboxamide,

(5) N-(4-benzoylphenyl)-3,4-dihydro-3-oxospiro[isoquinoline-1(2H),4′-piperidine]-1′-carboxamide,

(6) 3,4-dihydro-3-oxo-N-(5-phenyl-2pyrazinyl)spiro-[isoquinoline-1(2H),4′-piperidine)-1-carboxamide,

(7) 3,4-dihydro-N-(7-methyl-2-quinolyl)-3-oxospiro-[isoquinoline-1(2H),4′-piperidine]-1-carboxamide,

(8) N-(4-acetylphenyl)-3,4-dihydro-3-oxospiro-[isoquinoline-1(2H),4′-piperidine]-1′-carboxamide,

(9) 3,4-dihydro-3-oxo-N-[1-(2-quinolyl)-4-imidazolyl)-spiro[isoquinoline-1(2H),4′-piperidine]-1′-carboxamide,

(10) 3,4-dihydro-3-oxo-N-(5-oxo-5,6,7,8-tetrahydro-2-naphthyl)spiroisoquinoline-(2H),4′-piperidine]-1′-carboxamide,

(11) 3,4-dihydro-N-[5-(2-methyl-1-propenyl)-2-pyrazinyl]-3-oxospiro[isoquinoline-1(2H),4′-piperidine-1′-carboxamide,

(12) 3,4-dihydro-3-oxo-N-(3-phenyl-5-isoxazolyl)spiro-[isoquinoline-1(2H),4′-piperidine]-1′-carboxamide,

(13) N-[1-(7-benzo[b]furanyl)-4-imidazolyl]-3,4-dihydro-3-oxospiro[isoquinoline-1(2H),4′-piperidine]-1′-carboxamide,

(14) N-[-(3-difluoromethoxyphenyl)-4-imidazolyl]-3,4-dihydro-3-oxospiro[isoquinoline-1(2H),4′-piperidine]-1′-carboxamide,

(15) 3,4-dihydro-3-oxo-N-[4-(2-pyridylcarbonyl)phenyl]-spiro[isoquinoline-(2H),4′-piperidine]-1′-carboxamide,

(16) N-(3,4-dichlorophenyl)-3,4-dihydro-3-oxospiro-[isoquinoline-1(2H),4′-piperidine)-1′-carboxamide,

(17) N-[1-(3-chlorophenyl)-4-imidazolyl]-3,4-dihydro-3-oxospiro[isoquinoline-1(2H),4′-piperidine]-1′-carboxamide,

(18) 3,4-dihydro-3-oxo-N-(5-phenyl-2-thiazolyl)spiro-[isoquinoline-1(2H),4′-piperidine]-1′-carboxamide,

(19) 3,4-dihydro-3-oxo-N-[5-(2-pyridyl)-2-pyrazinyl]spiro-[isoquinoline-1(2H),4′-piperidine]-1′-carboxamide,

(20) 3,4-dihydro-N-(4-methyl-2-benzothiazolyl)-3-oxospiro-[isoquinoline-1(2H),4′-piperidine]-1′-carboxamide,

(21) N-(5-chloro-2-benzoxazolyl)-3,4-dihydro-3-oxospiro-[isoquinoline-1(2H),4′-piperidine]-1′-carboxamide,

(22) N-(4-benzoylphenyl)-3-oxospiro[isobenzofuran-1(3H),4′-piperidine]-1′-carboxamide,

(23) 3-oxo-N-(5-phenyl-2-pyrazinyl)-spiro[isobenzofuran-1(3H),4′-piperidine]-1′-carboxamide,

(24) N-(7-methyl-2-quinolyl)-3-oxospiro[isobenzofuran-1(3H),4′-piperidine]-1′-carboxamide,

(25) 3-oxo-N-(3-phenyl-5-isoxazolyl)spiro[isobenzofuran-1(3H),4′-piperidine]-1′-carboxamide,

(26) 3-oxo-N-(7-trifluoromethylpyrido[3,2-b]pyridin-2-yl)spiro[isobenzofuran-1(3H),4′-piperidine]-1′-carboxamide,

(27) 3-oxo-N-(5-phenyl-2-pyrimidinyl)spiro[isobenzofuran-1(3H),4′-piperidine]-1′-carboxamide,

(28) 3-oxo-N-[1-(3-quinolyl)-4-imidazolyl]spiro[isobenzofuran-1(3H),4′-piperidine)-1′-carboxamide,

(29) 3-oxo-N-(5-phenyl-3-pyrazolyl)spiro[isobenzofuran-1(3H),4′-piperidine]-1′-carboxamide,

(30) N-[5-(4-chlorophenyl)-3-pyrazolyl]-3-oxospiro-[isobenzofuran-1(3H),4′-piperidine]-1′-carboxamide,

(31) 3-oxo-N-[5-(3-quinolyl)-3-pyrazolyl]spiro[isobenzofuran-1(3H),4′-piperidine]-1′-carboxamide,

(32) N-[5-(3-fluorophenyl)-2-pyrimidinyl]-3-oxospiro-[isobenzofuran-1(3H),4′-piperidine]-1′-carboxamide,

(33) 3-oxo-N-[5-(3-trifluoromethylphenyl)-2-pyrimidinyl]-spiro[isobenzofuran-1(3H),4′-piperidine]-1′-carboxamide,

(34) N-[5-(3-chlorophenyl)-2-pyrimidinyl]-3-oxospiro-[isobenzofuran-1(3H),4′-piperidine]-1′-carboxamide,

(35) N-(7-difluoromethoxypyrido[3,2-b]pyridin-2-yl)-3-oxospiro[isobenzofuran-1(3H),4′-piperidine]-1′-carboxamide,

(36) 3-oxo-N-(5-phenyl-1,2,4-thiadiazol-3-yl)spiro-[isobenzofuran-1(3H),4′-piperidine]-1′-carboxamide,

(37) N-{1-[3-(2-hydroxyethyl)phenyl]4-imidazolyl}-3-oxospiro-[isobenzofuran-1(3H),4′-piperidine]-1′-carboxamide,

(38) N-[4-(1-ethyl-2-imidazolyl)phenyl]-3-oxospiro-[isobenzofuran-1(3H),4′-piperidine]-1′-carboxamide,

(39) N-[1-(3-methoxyphenyl)-4-imidazolyl]-3-oxospiro-[isobenzofuran-1(3H),4′-piperidine]-1′-carboxamide,

(40) 6-fluoro-3-oxo-N-(5-phenyl-2-pyrazinyl)spiro-[isobenzofuran-1(3H),4′-piperidine]-1′-carboxamide,

(41) 6-fluoro-3-oxo-N-(5-phenyl-2-pyrimidinyl)spiro-[isobenzofuran-1(3H),4′-piperidine]-1′-carboxamide,

(42) 5-fluoro-3-oxo-N-(5-phenyl-2-pyrazinyl)spiro[isobenzofuran-1(3H),4′-piperidine]-1′-carboxamide,

(43) 5-fluoro-3-oxo-N-(5-phenyl-2-pyrimidinyl)spiro-[isobenzofuran-1(3H),4′-piperidine]-1′-carboxamide,

(44) N-(4-benzoylphenyl)-3,4-dihydro-3-oxospiro[1H-2-benzopyran-1,4′-piperidine]-1′-carboxamide,

(45) 3,4-dihydro-3-oxo-N-(5-phenyl-2-pyrazinyl)spiro[1H-2-benzopyran-1,4′-piperidine]-1′-carboxamide,

(46) N-(5-benzoyl-2-pyrazinyl)-3,4-dihydro-3-oxospiro[1H-2-benzopyran-1,4′-piperidine]-1′-carboxamide,

(47) trans-N-(4-benzoylphenyl)-3′-oxospiro[cyclohexane-1,1′(3′H)-isobenzofuran]4-carboxamide,

(48) trans-3′-oxo-N-(5-phenyl-2-pyrazinyl)spiro[cyclohexane-1,1′(3′H)-isobenzofuran]4-carboxamide,

(49) trans-3′-oxo-N-(1-phenyl-4-imidazolyl)spiro[cyclohexane-1,1′(3′H)-isobenzofuran]-4-carboxamide,

(50) trans-3′-oxo-N-(5-phenyl-2-pyrimidinyl)spiro[cyclohexane-1,1′(3′H)-isobenzofuran]4-carboxamide,

(51) trans-N-[1-(3,5-difluorophenyl)4-imidazolyl]-3′-oxospiro-[cyclohexane-1,1′(3′H)-isobenzofuran]-4-carboxamide,

(52) trans-3′-oxo-N-(5-phenyl-3-pyrazolyl)spiro[cyclohexane-1,1′(3′H)-isobenzofuran]4-carboxamide,

(53) trans-N-[1-(2-fluorophenyl)-4-imidazolyl]-3′-oxospiro-[cyclohexane-1,1′(3′H)-isobenzofuran]-4-carboxamide,

(54) trans-N-(4-acetyl-3-trifluoromethylphenyl)-3′-oxospiro-[cyclohexane-1,1′(3′H)-isobenzofuran)-4-carboxamide,

(55) trans-3′-oxo-N-[1-3-quinolyl)-4-imidazolyl)-spiro[cyclohexane-1,1′(3′H)-isobenzofuran]-4-carboxamide,

(56) trans-N-[1-(3-cyanophenyl)-4-imidazolyl]-3′-oxospiro-[cyclohexane-1,1′(3′H)-isobenzofuran]-4-carboxamide,

(57) trans-N-(4-benzoylphenyl)-3-oxospiro[4-azaisobenzofuran-1(3H),1′-cyclohexane]-4′-carboxamide,

(58) trans-3-oxo-N-(5-phenyl-2-pyrazinyl)spiro[4-azaisobenzofuran-1(3H),1′-cyclohexane]-4′-carboxamide,

(59) trans-3-oxo-N-(3-phenyl-5-isoxazolyl)spiro[4-azaisobenzofuran-1(3H), 1′-cyclohexane]-4′-carboxamide,

(60) trans-3-oxo-N-(5-phenyl-2-pyrimidinyl)spiro[4-azaisobenzofuran-(3H),1′-cyclohexane]4′-carboxamide,

(61) trans-N-(4-benzoylphenyl)-3-oxospiro[5-azaisobenzofuran-1(3H),1′-cyclohexane]-4′-carboxamide,

(62) trans-N-(4-benzoylphenyl)-3-oxospiro[6-azaisobenzofuran-1(3H),1′-cyclohexane]4′-carboxamide,

(63) N-[5-(4-hydroxyphenyl)-2-pyrazinyl]-3-oxospiro[isobenzofuran-1(3H),4′-piperidine)-1′-carboxamide,

(64) N-[5-(3-hydroxyphenyl)-2-pyrazinyl]-3-oxospiro[isobenzofuran-1(3H),4′-piperidine]-1′-carboxamide,

(65) 4-fluoro-3-oxo-N-(5-phenyl-2-pyrimidinyl)spiro[isobenzofuran-1(3H),4′-piperidine]-1′-carboxamide,

(66) 7-fluoro-3-oxo-N-(5-phenyl-2-pyrimidinyl)spiro[isobenzofuran-1(3H),4′-piperidine]-1′-carboxamide,

(67) 6-ethyl-3-oxo-N-(5-phenyl-2-pyrazinyl)spiro[isobenzofuran-1(3H),4′-piperidine]-1′-carboxamide,

(68) 6-hydroxy-3-oxo-N-(5-phenyl-2-pyrazinyl)spiro[isobenzofuran-1(3H),4′-piperidine]-1′-carboxamide,

(69) trans-3-oxo-N-(5-phenyl -2-pyrimidinyl)spiro[5-azaisobenzofuran-1(3H),1′-cyclohexane]4′-carboxamide,

(70) trans-N-[5-(3-fluorophenyl)-2-pyrimidinyl]-3-oxospiro[5-azaisobenzofuran-1(3H),1′-cyclohexane]4′-carboxamide,

(71) trans-N-[5-(2-fluorophenyl)-2-pyrimidinyl]-3-oxospiro[5-azaisobenzofuran-1(3H),1′-cyclohexane]4′-carboxamide,

(72) trans-3-oxo-N-(4-phenyl-2-oxazolyl)spiro[5-azaisobenzofuran-1(3H),1′-cyclohexane]-4′-carboxamide,

(73) trans-N-[5-(2-methylphenyl)-2-pyrimidinyl]-3-oxospiro[5-azaisobenzofuran-1(3H),1′-cyclohexane]4′-carboxamide,

(74) trans-N-[5-(3-methylphenyl)-2-pyridinyl-3-oxospiro[5-azaisobenzofuran-1(3H),1′-cyclohexane]4′-carboxamide,

(75) trans-N-[5-(3-fluoromethoxyphenyl)-2-pyrimidinyl]-3-oxospiro[5-azaisobenzofuran-1(3H),1′-cyclohexane]-4′-carboxamide,

(76) trans-N-[5-(3-fluoromethylphenyl)-2-pyrimidinyl]-3-oxospiro[5-azaisobenzofuran-1(3H), 1′-cyclohexane]-4′-carboxamide,

(77) trans-N-[5-(3-fluoro-5-methoxyphenyl)-2-pyrimidinyl]-3-oxospiro[5-azaisobenzofuran-1(3H),1′-cyclohexane]-4′-carboxamide,

(78) trans-N-[5-(2-fluoro-5-methylphenyl)-2-pyrimidinyl]-3-oxospiro[5-azaisobenzofuran-1(3H),1′-cyclohexane]-4′-carboxamide,

(79) trans-N-[4-(3-fluoromethoxyphenyl)-2-oxazolyl]-3-oxospiro[5-azaisobenzofuran-1(3H),1′-cyclohexane]-4′-carboxamide,

(80) trans-N-[5-(3-hydroxymethylphenyl)-2-pyrimidinyl]-3-oxospiro[5-azaisobenzofuran-1(3H), 1′-cyclohexane]4′-carboxamide,

(81) trans-N-[5-(3-hydroxyphenyl)-2-pyrimidinyl]-3-oxospiro[5-azaisobenzofuran-1(3H),1′-cyclohexane]-4′-carboxamide,

(82) trans-3-oxo-N-(5-phenyl-2-pyrimidinyl)spiro[6-azaisobenzofuran-1(3H),1′-cyclohexane]-4′-carboxamide,

(83) trans-N-[5-(3-fluoromethylphenyl)-2-pyrimidinyl]-3-oxospiro[6-azaisobenzofuran-1(3H),1′-cyclohexane]-4′-carboxamide,

(84) trans-N-[5-(3-fluoromethoxyphenyl)-2-pyrimidinyl]-3-oxospiro[6-azaisobenzofuran-1(3H),1′-cyclohexane]-4′-carboxamide,

(85) trans-3-oxo-N-(6-phenyl-1,2,4-triazin-3-yl)spiro[6-azaisobenzofuran-1(3H),1′-cyclohexane]4′-carboxamide,

(86) trans-N-[5-(2-difluoromethoxyphenyl)-3-pyrazolyl]-3-oxospiro[6-azaisobenzofuran-1(3H),1′-cyclohexane]-4′-carboxamide,

(87) trans-N-[5-(3-difluoromethoxyphenyl)-3-pyrazolyl]-3-oxospiro[6-azaisobenzofuran-1(3H),1′-cyclohexane]4′-carboxamide,

(88) trans-N-[5-3-fluorophenyl)-3-pyrazolyl]-3-oxospiro[6-azaisobenzofuran-1(3H),1′-cyclohexane]-4′-carboxamide,

(89) trans-N-[5-(4-fluorophenyl)-3-pyrazolyl]-3-oxospiro[6-azaisobenzofuran-1(3H),1′-cyclohexane]-4′-carboxamide,

(90) trans-N-(4-benzoylphenyl)-3-oxospiro[7-azaisobenzofuran-1(3H),1′-cyclohexane]4′-carboxamide,

(91) trans-N-[1-(3,5-difluorophenyl)-4-imidazolyl]-3-oxospiro[7-azaisobenzofuran-1(3H),1′-cyclohexane]4′-carboxamide,

(92) trans-3-oxo-N-[2-phenyl-4-pyridyl]spiro[7-azaisobenzofuran-1(3H),1′-cyclohexane]-4′-carboxamide,

(93) trans-3-oxo-N-(1-phenyl-4-pyrazolyl)spiro[7-azaisobenzofuran-1(3H),1′-cyclohexane]-4′-carboxamide,

(94) trans-3-oxo-N-(1-phenyl-3-pyrrolyl)spiro[7-azaisobenzofuran-1(3H),1′-cyclohexane]4′-carboxamide,

(95) trans-N-[1-(4-fluorophenyl)-3-pyrazolyl]-3-oxospiro[7-azaisobenzofuran-1(3H),1′-cyclohexane]-4′-carboxamide,

(96) trans-3-oxo-N-(1-phenyl-3-pyrazolyl)spiro[4-azaisobenzofuran-1(3H),1′-cyclohexane]-4′-carboxamide,

(97) trans-3-oxo-N-(1-phenyl-4-pyrazolyl)spiro[4-azaisobenzofuran-1(3H),1′-cyclohexane]-4′-carboxamide,

(98) trans-N-[1-(3-fluorophenyl)-4-pyrazolyl]-3-oxospiro[4-azaisobenzofuran-1(3H),1′-cyclohexane]-4′-carboxamide,

(99) trans-3-oxo-N-(1-phenyl-3-pyrazolyl)spiro[6-azaisobenzofuran-1(3H),1′-cyclohexane]-4′-carboxamide,

(100) trans-N-[1-(4-fluorophenyl)-3-pyrazolyl]-3-oxospiro[6-azaisobenzofuran-1(3H),1′-cyclohexane]-4′-carboxamide,

(101) trans-N-[1-(2-fluorophenyl)-3-pyrazolyl]-3-oxospiro[6-azaisobenzofuran-1(3H),1′-cyclohexane]-4′-carboxamide,

(102) trans-3-oxo-N-(5-phenyl-1,2,4-thiadiazol-3-yl)spiro[6-azaisobenzofuran-1(3H),1′-cyclohexane]-4′-carboxamide,

(103) trans-3-oxo-N-(5-phenyl-3-isoxazolyl)spiro[6-azaisobenzofuran-1(3H),1′-cyclohexane]-4′-carboxamide,

(104) trans-3-oxo-N-(6-phenyl-3-pyridyl)spiro[6-azaisobenzofuran-1(3H),1′-cyclohexane]4′-carboxamide,

(105) trans-3-oxo-N-(2-phenyl-3-thiazolyl)spiro[6-azaisobenzofuran-1(3H),1′-cyclohexane]-4′-carboxamide,

(106) trans-3-oxo-N-(2-phenyl-1,2,3-triazol-4-yl)spiro[6-azaisobenzofuran-1(3H),1′-cyclohexane]4′-carboxamide,

and pharmaceutically acceptable salts and esters thereof.

In another sub-class of this class, the NPY5 antagonist is selected from the group consisting of

(1) trans-3-oxo-N-(5-phenyl-2-pyrimidinyl)spiro[4-azaisobenzofuran-1(3H),1′-cyclohexane]4′-carboxamide;

(2) trans-N-[5-(2-methylphenyl)-2-pyrimidinyl]-3-oxospiro[5-azaisobenzofuran-1(3H),1′-cyclohexane]-4′-carboxamide;

(3) trans-3-oxo-N-(5-phenyl-2-pyrimidinyl)spiro[6-azaisobenzofuran-1(3H),1′-cyclohexane]-4′-carboxamide;

(4) trans-3-oxo-N-(1-phenyl-4-pyrazolyl)spiro[7-azaisobenzofuran-1(3H),1′-cyclohexane]4′-carboxamide;

(5) trans-N-[1-(3-fluorophenyl)-4-pyrazolyl]-3-oxospiro[4-azaisobenzofuran-1(3H), 1′-cyclohexane]4′-carboxamide; and

(6) trans-N-[1-(2-fluorophenyl)-3-pyrazolyl]-3-oxospiro[6-azaisobenzofuran-1(3H),1′-cyclohexane-4′-carboxamide;

and pharmaceutically acceptable salts and esters thereof.

In another sub-class of this class, the NPY5 antagonist is trans-3-oxo-N-(5-phenyl-2-pyrimidinyl)spiro[4-azaisobenzofuran-1(3H),1′-cyclohexane]4′-carboxamide, and pharmaceutically acceptable salts and esters thereof.

In another sub-class of this class, the NPY5 antagonist is trans-N-[5-(2-methylphenyl)-2-pyrimidinyl]-3-oxospiro[5-azaisobenzofuran-1(3H),1′-cyclohexane]-4′-carboxamide, and pharmaceutically acceptable salts and esters thereof.

In another sub-class of this class, the NPY5 antagonist is trans-3-oxo-N-(5-phenyl-2-pyrimidinyl)spiro[6-azaisobenzofuran-1(3H),1′-cyclohexane]4′-carboxamide, and pharmaceutically acceptable salts and esters thereof.

In another sub-class of this class, the NPY5 antagonist is trans-3-oxo-N-(1-phenyl-4-pyrazolyl)spiro[7-azaisobenzofuran-1(3H),1′-cyclohexane]4′-carboxamide, and pharmaceutically acceptable salts and esters thereof.

In another sub-class of this class, the NPY5 antagonist is trans-N-[1-(3-fluorophenyl)-4-pyrazolyl]-3-oxospiro[4-azaisobenzofuran-1(3H),1′-cyclohexane]-4′-carboxamide, and pharmaceutically acceptable salts and esters thereof.

In another sub-class of this class, the NPY5 antagonist is trans-N-[1-(2-fluorophenyl)-3-pyrazolyl]-3-oxospiro[6-azaisobenzofuran-1(3H),1-cyclohexane]-4′-carboxamide, and pharmaceutically acceptable salts and esters thereof.

In yet another sub-class of this class, the NPY5 antagonist is 3-oxo-N-(5-phenyl-2-pyrazinyl)-spiro[isobenzofuran-1(3H),4′-piperidine-1′-carboxamide, and pharmaceutically acceptable salts and esters thereof.

The NPY5 antagonists of formula I and their preparation are disclosed in U.S. Pat. Nos. 6,326,375; 6,335,345; and International Publication No. WO 01/14376.

In another subclass of this class of the present invention, the NPY5 antagonist useful in the present invention is represented by the compound of structural Formula II: 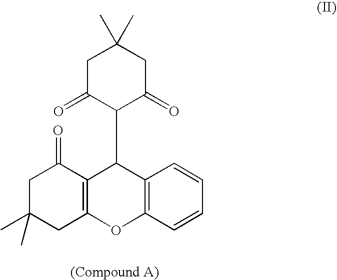
and pharmaceutically acceptable salts, esters and tautomers thereof.

The NPY5 antagonist of Formula II (Compound A) and its preparation are disclosed in J. Organic Chemistry, vol. 31, No. 5, p. 1639 (1966); and U.S. Pat. No. 6,258,837.

In another embodiment of the present invention, the anti-obesity agent is selected from the group consisting of: (1) aminorex; (2) amphechloral; (3) amphetamine; (4) benzphetamine; (5) bupropion; (6) chlorphentermine; (7) clobenzorex; (8) cloforex; (9) clominorex; (10) clortermine; (11) cyclexedrine; (12) dexfenfluramine; (13) dextroamphetamine; (14) diethylpropion; (15) diphemethoxidine; (16) N-ethylamphetamine; (17) fenbutrazate; (18) fenfluramine; (19) fenisorex; (20) fenproporex; (21) fludorex; (22) fluminorex; (23) furfurylmethylamphetamine; (24) levamfetamine; (25) levophacetoperane; (26) mazindol; (27) mefenorex; (28) metamfepramone; (29) methamphetamine; (30) norpseudoephedrine; (31) pentorex; (32) phendimetrazine; (33) phenmetrazine; (34) phentermine; (35) phenylpropanolamine; (36) picilorex; and (37) zonisamide; and pharmaceutically acceptable salts thereof.

In a class of this embodiment, the anti-obesity agent is selected from the group consisting of: dexfenfluramine, fenfluramine, and phentermine, and pharmaceutically acceptable salts thereof.

The methods and compositions of the present invention comprise an anti-dyslipidemic agent. The anti-dyslipidemic agent useful in the compositions of the present invention may be any agent useful to treat dyslipidemia known in the art. The anti-dyslipidemic agent may be peptidal or non-peptidal in nature, however, the use of a non-peptidal agent is preferred. For convenience, the use of an orally active anti-dyslipidemic agent is also preferred.

The anti-dyslipidemic agent useful in the compositions of the present invention is selected from the group consisting of:

(1) bile acid sequesterant,

(2) a HMG-CoA reductase inhibitor,

(3) a HMG-CoA synthase inhibitor,

(4) a cholesterol absorption inhibitor,

(5) an acyl coenzyme A-cholesterol acyl transferase (ACAT) inhibitor,

(6) a cholesteryl ester transfer protein (CETP) inhibitor,

(7) a squalene synthetase inhibitor,

(8) an anti-oxidant,

(9) a PPAR α agonist,

(10) a FXR receptor antagonist,

(11) a LXR receptor agonist,

(12) a lipoprotein synthesis inhibitor,

(13) a renin angiotensin system inhibitor,

(14) a microsomal triglyceride transport inhibitor,

(15) a bile acid reabsorption inhibitor,

(16) a PPARδ agonist,

(17) a triglyceride synthesis inhibitor,

(18) a transcription modulator,

(19) a squalene epoxidase inhibitor,

(20) a low density lipoprotein (LDL) receptor inducer,

(21) a platelet aggregation inhibitor,

(22) a 5-LO or FLAP inhibitor,

(23) a PPAR δ partial agonist, and

(24) niacin or a niacin receptor agonist,

and pharmaceutically acceptable salts and esters thereof.

In one embodiment of the present invention, the anti-dyslipidemic agent is selected from the group consisting of:

(1) bile acid sequesterant;

(2) a HMG-CoA reductase inhibitor;

(3) a HMG-CoA synthase inhibitor;

(4) a cholesterol absorption inhibitor;

(5) an acyl coenzyme A cholesterol acyl transferase (ACAT) inhibitor;

(6) a cholesteryl ester transfer protein inhibitor;

(7) a squalene synthetase inhibitor;

(8) an anti-oxidant,

(9) a PPARα agonist;

(10) a lipoprotein synthesis inhibitor; and

(11) a HMG-CoA reductase transcription inhibitor;

and pharmaceutically acceptable salts and esters thereof.

In another embodiment, the anti-dyslipidemic agent is a bile acid sequesterant, and pharmaceutically acceptable salts or esters thereof. In a class of this embodiment, the bile acid sequesterant is selected from the group consisting of cholestyramine, colestipol, and dialkylaminoalkyl derivatives of a cross-linked dextran; and pharmaceutically acceptable salts and esters thereof.

In another embodiment, the anti-dyslipideimic agent is a HMG-CoA reductase inhibitor, and pharmaceutically acceptable salts or esters thereof. In a class of this embodiment, the HMG-CoA reductase inhibitor is selected from the group consisting of atorvastatin, cerivastatin, itavastatin, fluvastatin, lovastatin, pravastatin, rivastatin, simvastatin, and ZD4522; and pharmaceutically acceptable salts and esters thereof. In a subclass of this class, the HMG-CoA reductase inhibitor is simvastatin; and pharmaceutically acceptable salts and esters thereof. In another subclass of this class, the HMG-CoA reductase inhibitor is lovastatin; and pharmaceutically acceptable salts and esters thereof.

In another embodiment, the antidyslipidemic agent is a HMG-CoA synthase inhibitor, and pharmaceutically acceptable salts or esters thereof.

In another embodiment, the anti-dyslipidemic agent is a cholesterol absorption inhibitor, and pharmaceutically acceptable salts or esters thereof. In a class of this embodiment, the cholesterol absorption inhibitor is selected from the group consisting of beta-sitosterol, ezetimibe, and tiqueside; and pharmaceutically acceptable salts and esters thereof. In a subclass of this class, the cholesterol absorption inhibitor is ezetimibe; and pharmaceutically acceptable salts and esters thereof.

In another embodiment, the anti-dyslipidemic agent is a composition comprising a cholesterol absorption inhibitor, and pharmaceutically acceptable salts or esters thereof; and a HMG-CoA reductase inhibitor, and pharmaceutically acceptable salts or esters thereof. In a class of this embodiment, the composition comprises ezetimibe, and pharmaceutically acceptable salts and esters thereof; and simvastatin, and pharmaceutically acceptable salts and esters thereof. In another class of this embodiment, the anti-dyslipidemic agent is vytorin®.

In another embodiment, the anti-dyslipidemic agent is an acyl coenzyme A-cholesterol acyl transferase (ACAT) inhibitor, and pharmaceutically acceptable salts or esters thereof. In a class of this embodiment, the acyl coenzyme A -cholesterol acyl transferase (ACAT) inhibitor is selected from the group consisting of avasimibe, eflucimibe, KY505 and SMP 797; and pharmaceutically acceptable salts and esters thereof.

In another embodiment, the anti-dyslipidemic agent is a cholesteryl ester transfer protein (CETP) inhibitor, and pharmaceutically acceptable salts or esters thereof.

In another embodiment, the anti-dyslipidemic agent is a squalene synthetase inhibitor, and pharmaceutically acceptable salts or esters thereof.

In another embodiment, the anti-dyslipidemic agent is an anti-oxidant, and pharmaceutically acceptable salts or esters thereof. In a class of this embodiment, the anti-oxidant is selected from the group consisting of probucol; and pharmaceutically acceptable salts and esters thereof.

In another embodiment, the anti-dyslipidemic agent is a PPAR α agonist, and pharmaceutically acceptable salts or esters thereof. In a class of this embodiment, the PPARα agonist is selected from the group consisting of beclofibrate, benzafibrate, ciprofibrate, clofibrate, etofibrate, fenofibrate, and gemfibrozil; and pharmaceutically acceptable salts and esters thereof.

In another embodiment, the anti-dyslipidemic agent is a lipoprotein synthesis inhibitor, and pharmaceutically acceptable salts or esters thereof. In a class of this embodiment, the lipoprotein synthesis inhibitor is selected from the group consisting of niacin or nicotinic acid; and pharmaceutically acceptable salts and esters thereof.

In another embodiment, the antidyslipidemic agent is a FXR receptor antagonist, and pharmaceutically acceptable salts or esters thereof.

In another embodiment, the anti-dyslipidemic agent is a LXR receptor agonist, and pharmaceutically acceptable salts or esters thereof.

In another embodiment, the anti-dyslipidemic agent is a renin angiotensin system inhibitor, and pharmaceutically acceptable salts or esters thereof.

In another embodiment, the anti-dyslipidemic agent is a PPARδ agonist, and pharmaceutically acceptable salts or esters thereof.

In another embodiment, the anti-dyslipidemic agent is a HMG-CoA reductase transcription inhibitor, and pharmaceutically acceptable salts or esters thereof.

In another embodiment, the anti-dyslipidemic agent is a triglyceride synthesis inhibitor, and pharmaceutically acceptable salts or esters thereof.

In another embodiment, the anti-dyslipidemic agent is a transcription modulator, and pharmaceutically acceptable salts or esters thereof.

In another embodiment, the anti-dyslipidemic agent is a microsomal triglyceride transport inhibitor, and pharmaceutically acceptable salts or esters thereof.

In another embodiment, the anti-dyslipidemic agent is a bile acid reuptake inhibitor, and pharmaceutically acceptable salts or esters thereof.

In another embodiment, the anti-dyslipidemic agent is a squalene epoxidase inhibitor, and pharmaceutically acceptable salts or esters thereof.

In another embodiment, the anti-dyslipidemic agent is a low density lipoprotein (LDL) receptor inducer, and pharmaceutically acceptable salts or esters thereof.

In another embodiment, the anti-dyslipidemic agent is a platelet aggregation inhibitor, and pharmaceutically acceptable salts or esters thereof.

In another embodiment, the anti-dyslipidemic agent is a 5-LO or FLAP inhibitor, and pharmaceutically acceptable salts or esters thereof.

In another embodiment, the anti-dyslipidemic agent is a PPAR δ partial agonist, and pharmaceutically acceptable salts or esters thereof.

In another embodiment, the anti-dyslipidemic agent is niacin or a niacin receptor agonist, and pharmaceutically acceptable salts or esters thereof.

The present invention relates to a method of treating, controlling, or preventing dyslipidemia in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling, or preventing hyperlipidemia in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling, or preventing low HDL cholesterol in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling, or preventing high LDL cholesterol in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling, or preventing hypertriglyceridemia in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling, or preventing dyslipidemia associated with obesity in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling, or preventing a dyslipidemia-related disorder in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling, or preventing hypercholesterolemia, atherosclerosis, low HDL cholesterol levels, high LDL cholesterol levels, hyperlipidemia, hypertriglyceridemia in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling or preventing hypercholesterolemia, in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling or preventing hypertriglyceridemia, in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling or preventing lipid disorders, hyperlipidemia, or low HDL, in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

Another aspect of the invention provides lipid management in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling or preventing atherosclerosis, in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

It is understood that the sequellae of atherosclerosis (angina, claudication, heart attack, stroke, etc.) are thereby treated.

The present invention relates to a method of treating, controlling or preventing the onset of atherosclerosis in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method for reducing atherosclerotic progression in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling or preventing atherosclerosis, in a subject in need thereof comprising administration of
(a) a therapeutically effective amount of an NPY5 antagonist, and pharmaceutically acceptable salts and esters thereof; and
(b) a therapeutically effective amount of a HMG-CoA reductase inhibitor selected from the group consisting of atorvastatin, cerivastatin, itavastatin, fluvastatin, lovastatin, pravastatin, rivastatin, simvastatin, and ZD4522, and pharmaceutically acceptable salts and esters thereof;
   to a subject in need of such treatment.

The present invention relates to a method of treating, controlling or preventing atherosclerosis, in a subject in need thereof comprising administration of
(a) a therapeutically effective amount of an NPY5 antagonist, and pharmaceutically acceptable salts and esters thereof; and
(b) a therapeutically effective amount of a cholesterol absorption inhibitor selected from the group consisting of beta-sitosterol, ezetimibe, and tiqueside, and pharmaceutically acceptable salts and esters thereof;
   to a subject in need of such treatment.

The present invention relates to a method of treating, controlling or preventing atherosclerosis, in a subject in need thereof comprising administration of
(a) a therapeutically effective amount of an NPY5 antagonist, and pharmaceutically acceptable salts and esters thereof; and
(b) a therapeutically effective amount of a bile acid sequesterant selected from the group consisting of cholestyramine, colestipol, and dialkylaminoalkyl derivatives of a cross-linked dextran, and pharmaceutically acceptable salts and esters thereof;
   to a subject in need of such treatment.

The present invention relates to a method of treating, controlling or preventing atherosclerosis, in a subject in need thereof comprising administration of
(a) a therapeutically effective amount of an NPY5 antagonist, and pharmaceutically acceptable salts and esters thereof; and
(b) a therapeutically effective amount of an acyl coenzyme A cholesterol acyl transferase (ACAT) inhibitor selected from the group consisting of avasimibe, eflucimibe, KYS05, and SMP 797, and pharmaceutically acceptable salts and esters thereof;
   to a subject in need of such treatment.

The present invention relates to a method of treating, controlling or preventing atherosclerosis, in a subject in need thereof comprising administration of
(a) a therapeutically effective amount of an NPY5 antagonist, and pharmaceutically acceptable salts and esters thereof; and
(b) a therapeutically effective amount of an anti-oxidant selected from the group consisting of probucol, and pharmaceutically acceptable salts and esters thereof;
   to a subject in need of such treatment.

The present invention relates to a method of treating, controlling or preventing atherosclerosis, in a subject in need thereof comprising administration of
(a) a therapeutically effective amount of an NPY5 antagonist, and pharmaceutically acceptable salts and esters thereof; and
(b) a therapeutically effective amount of a PPAR α agonist selected from the group consisting of beclofibrate, benzafibrate, ciprofibrate, clofibrate, etofibrate, fenofibrate, and gemfibrozil, and pharmaceutically acceptable salts and esters thereof;
   to a subject in need of such treatment.

The present invention relates to a method of treating, controlling or preventing atherosclerosis, in a subject in need thereof comprising administration of
(a) a therapeutically effective amount of an NPY5 antagonist, and pharmaceutically acceptable salts and esters thereof; and
(b) a therapeutically effective amount of a lipoprotein synthesis inhibitor selected from the group consisting of niacin and nicotinic acid, and pharmaceutically acceptable salts and esters thereof;
   to a subject in need of such treatment.

The present invention relates to a method of treating, controlling or preventing cardiac hypertrophy, particularly left ventricular hypertrophy, in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating, controlling or preventing metabolic syndrome in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention also relates to a method of treating, controlling or preventing metabolic syndrome in a subject in need thereof comprising administration of
(a) a therapeutically effective amount of an anti-obesity agent, and pharmaceutically acceptable salts and esters thereof;
(b) a therapeutically effective amount of an anti-dyslipidemic agent, and pharmaceutically acceptable salts and esters thereof; and
(c) a therapeutically effective amount of an anti-hypertensive and/or a therapeutically effective amount of an anti-diabetic agent;
to a subject in need of such treatment.

The present invention relates to a method of treating or preventing obesity in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
   to a subject in need of such treatment.

The present invention relates to a method of treating or preventing an o in a subject in need thereof comprising administration of
(a) a therapeutically effective amount of an anti-obesity agent, and pharmaceutically acceptable salts and esters thereof; and
(b) a therapeutically effective amount of an anti-dyslipidemic agent, and pharmaceutically acceptable salts and esters thereof;
to a subject in need of such treatment.

The present invention also relates to pharmaceutical compositions, and medicaments useful for carrying out these methods.

The present invention relates to the use of an anti-obesity agent, and pharmaceutically acceptable salts and esters thereof; and an anti-dyslipidemic agent, and pharmaceutically acceptable salts and esters thereof; for the manufacture of a medicament for treatment, control, or prevention of dyslipidemia which comprises an effective amount of an anti-obesity agent and an effective amount of an anti-dyslipidemic agent, together or separately.

The present invention relates to the use of an anti-obesity agent, and pharmaceutically acceptable salts and esters thereof; and an anti-dyslipidemic agent, and pharmaceutically acceptable salts and esters thereof; for the manufacture of a medicament for treatment, control or prevention of a dyslipidemia-related disorder which comprises an effective amount of an anti-obesity agent and an effective amount of an anti-dyslipidemic agent, together or separately.

The present invention relates to the use of an anti-obesity agent, and pharmaceutically acceptable salts and esters thereof; and an anti-dyslipidemic agent, and pharmaceutically acceptable salts and esters thereof; for the manufacture of a medicament for treatment, control, or prevention of obesity which comprises an effective amount of an anti-obesity agent and an effective amount of an anti-dyslipidemic agent, together or separately.

The present invention relates to the use of an anti-obesity agent, and pharmaceutically acceptable salts and esters thereof; and an anti-dyslipidemic agent, and pharmaceutically acceptable salts and esters thereof; for the manufacture of a medicament for treatment, control, or prevention of an obesity-related disorder which comprises an effective amount of an anti-obesity agent and an effective amount of an anti-dyslipidemic agent, together or separately.

The present invention relates to the use of an anti-obesity agent, and pharmaceutically acceptable salts and esters thereof; and an anti-dyslipidemic agent, and pharmaceutically acceptable salts and esters thereof; for the manufacture of a medicament for treatment, control, or prevention of metabolic syndrome which comprises an effective amount of an anti-obesity agent and an effective amount of an anti-dyslipidemic agent, together or separately.

The present invention relates to the use of an anti-obesity agent, and pharmaceutically acceptable salts and esters thereof; and an anti-dyslipidemic agent, and pharmaceutically acceptable salts and esters thereof; and one or more anti-diabetic agents and/or anti-hypertensive agents, for the manufacture of a medicament for treatment, control, or prevention of metabolic syndrome which comprises an effective amount of an anti-obesity agent and an effective amount of an anti-dyslipidemic agent, and an anti-diabetic agent and/or an anti-hypertensive agent, together or separately.

The present invention relates to the use of an anti-obesity agent, and pharmaceutically acceptable salts and esters thereof; and an anti-dyslipidemic agent, and pharmaceutically acceptable salts and esters thereof; for the manufacture of a medicament for treatment, control, or prevention of cardiac hypertrophy, particularly left ventricular hypertrophy, which comprises an effective amount of an anti-obesity agent and an effective amount of an anti-dyslipidemic agent, together or separately.

The present invention further relates to a product containing an anti-obesity agent, and pharmaceutically acceptable salts and esters thereof; and an anti-dyslipidemic agent, and pharmaceutically acceptable salts and esters thereof; as a combined preparation for simultaneous, separate or sequential use in dyslipidemia or a dyslipidemia-related disorder.

The present invention further relates to a product containing an anti-obesity agent, and pharmaceutically acceptable salts and esters thereof; and an anti-dyslipidemic agent, and pharmaceutically acceptable salts and esters thereof; as a combined preparation for simultaneous, separate or sequential use in obesity, or an obesity-related disorder.

The present invention further relates to a product containing an anti-obesity agent, and pharmaceutically acceptable salts and esters thereof; and an anti-dyslipidemic agent, and pharmaceutically acceptable salts and esters thereof; as a combined preparation for simultaneous, separate or sequential use in metabolic syndrome.

The present invention further relates to a product containing an anti-obesity agent, and pharmaceutically acceptable salts and esters thereof; and an anti-dyslipidemic agent, and pharmaceutically acceptable salts and esters thereof; and an anti-diabetic agent and/or an anti-hypertensive agent as a combined preparation for simultaneous, separate or sequential use in metabolic syndrome.

The present invention further relates to a product containing an anti-obesity agent, and pharmaceutically acceptable salts and esters thereof; and an anti-dyslipidemic agent, and pharmaceutically acceptable salts and esters thereof; as a combined preparation for simultaneous, separate or sequential use in cardiac hypertrophy, particularly left ventricular hypertrophy.

The invention further provides pharmaceutical compositions comprising an anti-obesity agent, and pharmaceutically acceptable salts and esters thereof, and an anti-dyslipidemic agent, and pharmaceutically acceptable salts and esters thereof, as active ingredients.

Anti-Obesity Agents

One of ordinary skill in the art can readily identify the agents useful in the compositions and methods of the present invention. Anti-obesity agents that are appetite suppressants can be evaluated in rodents according to the procedures described in: Daniels, A. J. et al., Regulatory Peptides, 106:47-54 (2002); Halaas, J. L. et. al., Science, 269: 543-546 (1995); and Strack, A. M., Obesity Research, 10:173-81 (2002). Anti-obesity agents that are metabolic rate enhancers are routinely evaluated in rodents (Atgie, C., Comp. Biochem. Physiol. A. Mol. Integr. Physiol. 119:629-36 (1998); Himms-Hagan, J., American J. Physiology, 266:R1371-82 (1994)), and, even when inactive in rodents, are tested in additional species such as dog and monkey before ultimately being tested in humans (Connacher, A. A. et. al., Int'l J. Obesity, 16: 685-694 (1992); Connacher, A. A. et. al., Am. J. Clin. Nutr., 55: 258S-261S (1992); Connacher, A. A. et. al., Brit. Med. J., 296: 1217-1220 (1998)). The utility of metabolic rate enhancers is supported by experiments with mice, in which the RII-beta gene has been deleted, that were shown to be resistant to diet induced obesity (D. E. Cummings et al. Nature 382: 622-626 (1996)). Anti-obesity agents that are nutrient absorption inhibitors can be evaluated in: Badr M. Z. and Chen, T. S., Toxicology, 34:333-40 (1985); Sorribas, V., J. Pharm. Pharmacol., 44:1030-2 (1992).

As used herein, the term “appetite suppressant” includes compounds that reduce total food intake by more than 5%, or reduce caloric intake or selectively reduce intake of specific components of the diet such as carbohydrates or fats by more than 5%. As used herein, the term “metabolic rate enhancer” includes compounds which, when administered to a subject, act to increase the metabolic rate of the subject, particularly those agents which increase metabolic rate by at least 5%, preferably 10%, most preferably 20% in 24 hour energy expenditure, and/or increase the oxidation of fatty acids relative to carbohydrates when administered to the subject. As used herein, the term “nutrient absorption inhibitor” includes compounds that inhibit the absorption of more than 10% of the nutrients.

Among the anti-obesity agents useful in the compositions of this invention are those identified, with dosing information, in the Physician's Desk reference, Edition 56 (2002). Generally, the anti-obesity agents are administered at a dosage ranging from about 0.0001 mg/kg to about 100 mg/kg of body weight, preferably from about 0.001 mg/kg to about 50 mg/kg of body weight. The term anti-obesity agent also includes within its meaning the compounds within the following therapeutic classes, or a combination of any of these compounds (e.g. a lipase inhibitor with an NPY5 antagonist):

Serotonin (5HT) transport inhibitors useful in this invention include, but are not limited to, paroxetine, fluoxetine, fenfluramine, fluvoxamine, sertraline, and imipramine.

Norepinephrine (NE) transport inhibitors useful in this invention include, but are not limited to, GW 320659, despiramine, talsupram, and nomifensine.

Cannabinoid receptor 1 (CB-1) antagonist/inverse agonists useful in the present invention include: U.S. Pat. Nos. 5,532,237, 4,973,587, 5,013,837, 5,081,122, 5,112,820, 5,292,736, 5,624,941 and U.S. Pat. No. 6,028,084; and PCT Application Nos. WO 96/33159, WO 98/33765, W098/43636, WO98/43635, WO 01/09120, WO98/31227, WO98/41519, WO98/37061, WO00/10967, WO00/10968, WO97/29079, WO99/02499, WO 01/58869, WO 02/076949, WO 01/64632, WO 01/64633, WO 01/64634, and WO 03/007887; and EPO Application No. EP-658546. Specific CB-1 antagonists/inverse agonists useful in the present invention include, but are not limited to, rimonabant (Sanofi Synthelabo), SR-147778 (Sanofi Synthelabo), BAY 65-2520 (Bayer), and SLV 319 (Solvay).

CCK-A agonists useful in the present invention include GI 181771, and SR 146,131.

Dipeptidyl peptidase IV (DP-IV) inhibitors, such as isoleucine thiazolidide; NVP-DPP728; P32198; and LAF 237, P 3298, TSL 225, valine pyrrolidide, TMC-2A/2B/2C, CD-26 inhibitors, FE 999011, P9310/K364, VIP 0177, DPP4, SDZ 274444; and the compounds disclosed in WO 03/004498 (16 Jan. 2003); WO 03/004496 (16 Jan. 2003); EP 1 258 476 (20 Nov. 2002); WO 02/083128 (24 Oct. 2002); WO 02/062764 (15 Aug. 2002); WO 03/000250 (3 Jan. 2003) 03/002530 (9 Jan. 2003); WO 03/002531 (9 Jan. 2003); WO 03/002553 (9 Jan. 2003); 03/002593 (9 Jan. 2003); WO 03/000180 (3 Jan. 2003); and WO 03/000181 (3 Jan. 2003).

Ghrelin antagonists useful in the present invention, include those in WO 01/87335, and WO 02/08250.

GLP-1 agonists useful in the present invention include exendin-3 and exendin-4, and those disclosed in US 2003087821 and NZ 504256.

Histamine 3 (H3) antagonist/inverse agonists useful in the present invention include: PCT Application No. WO 02/15905; and O-[3-(1H-imidazol4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm.(Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)). Specific H3 antagonists/inverse agonists useful in the present invention include, but are not limited to, thioperamide, 3-(1H-imidazol4-yl)propyl N-(4-pentenyl)carbamate, clobenpropit, iodophenpropit, imoproxifan, GT2394 (Gliatech), and A331440.

Melanin-concentrating hormone 1 receptor (MCH1R) antagonists and melanin-concentrating hormone 2 receptor (MCH2R) agonist/antagonists useful in the present invention include PCT Patent Application Nos. WO 01/82925, WO 01/87834, WO 02/06245, WO 02/04433, and WO 02/51809; and Japanese Patent Application No. JP 13226269. Specific MCH1R antagonists useful in the present invention include, but are not limited to, T-226296 (Takeda), SB 568849, and SNAP 7941.

Neuropeptide Y1 (NPY1) antagonists useful in the present invention, include: U.S. Pat. No. 6,001,836; and PCT Application Nos. WO 96/14307, WO 01/23387, WO 99/51600, WO 01/85690, WO 01/85098, WO 01/85173, and WO 01/89528. Specific examples of NPY1 antagonists useful in the present invention include, but are not limited to, BIBP3226, J-115814, BIBO 3304, LY-357897, CP-671906, and GI-264879A.

Neuropeptide Y2 (NPY2) agonists useful in the present invention, include, but are not limited to, peptide YY (PYY), and PYY3-36, peptide YY analogs, PYY agonists, and the compounds disclosed in WO 03/026591, WO 03/057235, and WO 03/027637.

Neuropeptide Y5 (NPY5) antagonists useful in the present invention, include, but are not limited to, the compounds described in: U.S. Pat. Nos. 6,140,354, 6,191,160, 6,258,837, 6,313,298, 6,337,332, 6,329,395, and 6,340,683; U.S. Pat. Nos. 6,326,375; 6,329,395; 6,337,332; 6,335,345; European Patent Nos. EP-01010691, and EP-01044970; and PCT International Patent Publication Nos. WO 97/19682, WO 97/20820, WO 97/20821, WO 97/20822, WO 97/20823, WO 98/27063, WO 00/107409, WO 00/185714, WO 00/185730, WO 00/64880, WO 00/68197, WO 00/69849, WO 01/09120, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/20488, WO 02/22592, WO 02/48152, WO 02/49648, and WO 01/14376. Specific NPY 5 antagonists useful in the combinations of the present invention, include, but are not limited to GW-569180A, GW-594884A, GW-587081X, GW-548118X; FR 235,208; FR226928, FR 240662, FR252384; 1229U91, GI-264879A, CGP71683A, LY-377897, LY366377, PD-160170, SR-120562A, SR-120819A, JCF-104, and H409/22. Additional specific NPY 5 antagonists useful in the combinations of the present invention, include, but are not limited to the compounds described in Norman et al., J. Med. Chem. 43:4288-4312 (2000).

Leptin includes, but is not limited to, recombinant human leptin (PEG-OB, Hoffman La Roche) and recombinant methionyl human leptin (Amgen). Leptin derivatives (e.g., truncated forms of leptin) useful in the present invention include: U.S. Pat. Nos. 5,552,524; 5,552,523; 5,552,522; 5,521,283; and PCT International Publication Nos. WO 96/23513; WO 96/23514; WO 96/23515; WO 96/23516; WO 96/23517; WO 96/23518; WO 96/23519; and WO 96/23520.

Opioid antagonists useful in the present invention include: PCT Application No. WO 00/21509. Specific opioid antagonists useful in the present invention include, but are not limited to, nalmefene (Revex®), 3-methoxynaltrexone, naloxone, and naltrexone.

Orexin antagonists useful in the present invention include: PCT Patent Application Nos. WO 01/96302, WO 01/68609, WO 02/51232, WO 02/51838, and WO 03/023561. Specific orexin antagonists useful in the present invention include, but are not limited to, SB-334867-A.

Acyl-estrogens useful in the present invention include oleoyl-estrone (del Mar-Grasa, M. et al., Obesity Research, 9:202-9 (2001)).

Cholecystokinin-A (CCK-A) agonists useful in the present invention include U.S. Pat. No. 5,739,106. Specific CCK-A agonists include, but are not limited to, AR-R 15849, GI 181771, JMV-180, A-71378, A-71623 and SR146131.

Specific ciliary neurotrophic factors (CNTF) useful in the present invention include, but are not limited to, GI-181771 (Glaxo-SmithKline); SR146131 (Sanofi Synthelabo); butabindide; PD170,292, PD 149164 (Pfizer). CNTF derivatives useful in the present invention include, but are not limited to, axokine (Regeneron); and PCT Application Nos. WO 94/09134, WO 98/22128, and WO 99/43813.

Growth hormone secretagogue (GHS) agonists useful in the present invention include: U.S. Pat. No. 6358951, and U.S. Patent Application Nos. 2002/049196 and 2002/022637; and PCT Application Nos. WO 01/56592, and WO 02/32888. Specific GHS agonists include, but are not limited to, NN703, hexarelin, MK-0677, SM-130686, CP-424,391, L-692,429 and L-163,255.

HT2c agonists useful in the present invention include: U.S. Pat. No. 3,914,250; and PCT Application Nos. WO 02/36596, WO 02/48124, WO 02/10169, WO 01/66548, WO 02/44152; WO 02/51844, WO 02/40456, and WO 02/40457. Specific 5HT2c agonists useful in this invention include, but are not limited to, BVT933, DPCA37215, IK264; PNU 22394; WAY161503, R-1065, and YM 348.

Mc4r agonists useful in the present invention include: PCT Application Nos. WO 99/64002, WO 00/74679, WO 01/991752, WO 01/74844, WO 01/70708, WO 01/70337, WO 01/91752, WO 02/059095, WO 02/059107, WO 02/059108, WO 02/059117, WO 02/12166, WO 02/11715, WO 02/12178, WO 02/15909, WO 02/068387, WO 02/068388, WO 02/067869, WO 03/007949, and WO 03/009847. Specific Mc4r agonists useful in the present invention include CHIR86036 (Chiron); ME-10142, and ME-10145 (Melacure).

Monoamine reuptake inhibitors useful in the present invention include: PCT Application Nos. WO 01/27068, and WO 01/62341. Specific monoamine reuptake inhibitors useful in the present invention include, but are not limited to, sibutramine (Meridia®/Reductil®) disclosed in U.S. Pat. Nos. 4,746,680, 4,806,570, and 5,436,272, and U.S. Patent Publication No. 2002/0006964. The present invention encompasses sibutratmine as a racemic mixture, as optically pure isomers (+) and (−), or a pharmaceutically acceptable salt, solvent, hydrate, clathrate or prodrug thereof; particularly sibutramine hydrochloride monohydrate.

Phytopharm compound 57 is also known as CP 644,673.

Serotonin reuptake inhibitors, and releasers, useful in the present invention include: dexfenfluramine, fluoxetine, and other serotonin reuptake inhibitors, including, but not limited to, those in U.S. Pat. No. 6,365,633; and PCT Patent Application Nos. WO 01/27060, and WO 01/162341.

11β HSD-1 inhibitor useful in the present invention include, but are not limited to, BVT 3498, BVT 2733, and those compounds disclosed in WO 01/90091, WO 01/90090, WO 01/90092, which are incorporated by reference herein in their entirety.

Uncoupling Protein (UCP-1, UCP-2, and UCP-3) activators useful in the present invention include: PCT Patent Application No. WO 99/00123. Specific uncoupling protein (UCP-1, UCP-2, and UCP-3) activators useful in the present invention include, but are not limited to, phytanic acid, 4-[(E)-2-(5,6,7,8-tetrahydro-5,5,8,8-tetramethyl-2-napthalenyl)-1-propenyl]benzoic acid (TTNPB), and retinoic acid.

β3 adrenergic receptor (β3) agonists useful in the present invention include: U.S. patent application Nos. 5,705,515, and U.S. Pat. No. 5,451677; and PCT Patent Application Nos. WO 01/74782, and WO 02/32897. Specific β3 agonists useful in the present invention include, but are not limited to, AD9677/TAK677 (Dainippon/Takeda), CL-316,243, SB 418790, BRL-37344, L-796568, BMS-196085, BRL-35135A, CGP12177A, BTA-243, GW 427353, Trecadrine, Zeneca D7114, and SR 59119A.

Thyroid hormone β agonists useful in the present invention include: PCT Application No. WO 02/15845; and Japanese Patent Application No. JP 2000256190. Specific thyroid hormone β agonists useful in the present invention include, but are not limited to, KB-2611 (KaroBioBMS).

Specific fatty acid synthase (FAS) inhibitors useful in the present invention, include, but are not limited to, Cerulenin and C75.

Specific phosphodieterase (PDE) inhibitors useful in the present invention, include, but are not limited to, theophylline, pentoxifylline, zaprinast, sildenafil, amrinone, milrinone, cilostamide, rolipram, and cilomilast.

Lipase inhibitors useful in the present invention include, but are not limited to, those disclosed in PCT Application No. WO 01/77094, and U.S. Pat. Nos. 4,598,089. 4,452,813, 5,512,565, 5,391,571, 5,602,151, 4,405,644, 4,189,438, and 4,242,453. Specific lipase inhibitors useful in the present invention include, but are not limited to, tetrahydrolipstatin (orlistat/Xenical®), Triton WR1339, RHC80267, lipstatin, teasaponin, and diethylumbelliferyl phosphate, FL-386, WAY-121898, Bay-N-3176, valilactone, esteracin, ebelactone A, ebelactone B, RHC 80267, stereoisomers thereof, and pharmaceutically acceptable salts of said compounds and stereoisomers.

Topiramate (Topimax®), indicated as an anti-convulsant and an anti-convulsant, has been shown to increase weight loss.

Zonisamide (Zonegran®), indicated as an anti-pileptic, has been shown to lead to weight loss.

Anti-Dyslipidemic Agents

One of ordinary skill in the art can readily identify the anti-dyslipidemic agents useful in the compositions and methods of the present invention. Anti-dystipidemic agents can be evaluated in a guinea pig model according to the procedures described in: Sullivan, M. et al., Lab. Anim. Sci. 43:575-778 (1993); Aoki, T., et al., Arzneimittelforschung 51:197-203, (2001).

Anti-dyslipidemic compounds useful in this invention, with dosing information, are identified in the Physician's Desk reference, Edition 56 (2002). Generally, the anti-dyslipidemic agents are administered at a dosage ranging from about 0.0001 mg/kg to about 100 mg/kg of body weight, preferably from about 0.001 mg/kg to about 50 mg/kg of body weight. The term “anti-dyslipidemic agent” includes within its meaning the compounds within the following therapeutic classes, or a combination of any of these compounds (e.g. a HMG-CoA reductase inhibitor with a cholesterol absorption inhibitor):

(1) a bile acid sequesterant,

(2) a HMG-CoA reductase inhibitor,

(3) a HMG-CoA synthase inhibitor,

(4) a cholesterol absorption inhibitor,

(5) an acyl coenzyme A-cholesterol acyl transferase (ACAT) inhibitor,

(6) a cholesteryl ester transfer protein (CETP) inhibitor,

(7) a squalene synthetase inhibitor,

(8) an anti-oxidant,

(9) a PPAR α agonist,

(10) a FXR receptor modulator,

(11) a LXR receptor agonist,

(12) a lipoprotein synthesis inhibitor,

(13) a renin angiotensin system inhibitor,

(14) a microsomal triglyceride transport inhibitor,

(15) a bile acid reabsorption inhibitor,

(16) a PPARδ agonist,

(17) a triglyceride synthesis inhibitor,

(18) a transcription modulator,

(19) a squalene epoxidase inhibitor,

(20) a low density lipoprotein (LDL) receptor inducer, and

(21) a platelet aggregation inhibitor,

(22) a 5-LO or FLAP inhibitor,

(23) a PPAR δ partial agonist,

(24) niacin or a niacin receptor agonist,

and pharmaceutically acceptable salts and esters thereof.

Bile acid sequestrants useful in the present invention include, but are not limited to, cholestyramine, colesevelem, colestipol (Colestid®), dialkylaminoalkyl derivatives of a cross-linked dextran; LoCholest®; and Questran®.

HMG-CoA reductase inhibitors useful in the present invention include, but are not limited to, atorvastatin (Lipitor®), cerivastatin (Baycol®), itavastatin, fluvastatin (Lescol®), lovastatin (Mevacor®), pravastatin (Pravachol®), rivastatin, simvastatin (Zocor®), and ZD4522; and those disclosed in U.S. Pat. No. 4,231,938, U.S. Pat. No. 4,444,784, U.S. Pat. No. 4,739,073, U.S. Pat. No. 4,346, 227; EP 491,226, and U.S. Pat. No. 4,647,576.

HMG-CoA synthase inhibitors useful in the present invention include, but are not limited to, those disclosed in U.S. Pat. No. 5,120,729, U.S. Pat. No. 5,064,856, and U.S. Pat. No. 4,847,271.

Cholesterol absorption inhibitors useful in the present invention include, but are not limited to, stanol esters, beta-sitosterol; sterol glycosides such as tiqueside; and azetidinones such as ezetimibe; and those disclosed in U.S. Pat. No. 5,846,966, U.S. Pat. No. 5,631,365, U.S. Pat. No. 5,767,115, U.S. Pat. No. 6,133,001, U.S. Pat. No. 5,886,171, U.S. Pat. No. 5,856,473, U.S. Pat. No. 5,756,470, U.S. Pat. No. 5,739,321, U.S. Pat. No. 5,919,672, WO 00/63703, WO /0060107, WO 00/38725, WO 00/34240, WO 00/20623, WO 97/45406, WO 97/16424, WO 97/16455, and WO 95/08532, the entire contents of which are hereby incorporated by reference. An examplary cholesterol absorption inhibitor is ezetimibe, which is 1-(4-fluorophenyl)-3(R)-(3(S)-hydroxy-3-(4-fluorophenyl)propyl]-4-(S)-(4-hydroxyphenyl)-2-azetidinone, shown below as

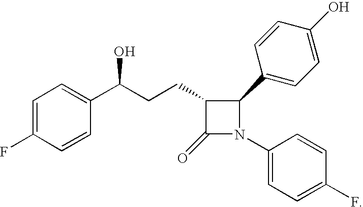

Additional examplary hydroxy-substituted azetidinone cholesterol absorption inhibitors are specifically described in U.S. Pat. No. 5,767,115, column 39, lines 54-61 and column 40, lines 1-51 (hereby incorporated by reference), represented by the formula 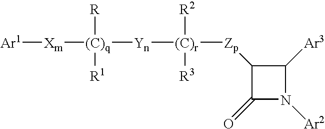
as defined in column 2, lines 20-63 (hereby incorporated by reference).

Acyl coenzyme A-cholesterol acyl transferase (ACAT) inhibitors useful in the present invention include, but are not limited to, avasimibe, eflucimibe, KY505, SMP 797, and the like; and those disclosed in U.S. Pat. No. 5,510,379, and International Patent Application Nos. WO 96/26948 and WO 96/10559.

CETP inhibitors useful in the present invention include, but are not limited to, JTT 705, torcetrapib, CP 532,632, BAY63-2149, SC 591, SC 795, and the like, and those disclosed in U.S. Pat. No. 5,512, 548; WO 99/20302, WO 99/14204, WO 99/41237, WO 95/04755, WO 96/15141, WO 96/05227, EP 796846, EP818197, EP 818448, DE 19704244, DE19741051, DE 19741399, DE 19704243, DE 19709125, DE 19627430, DE 19832159, DE 19741400, JP 11049743, and JP 09059155; and those disclosed in J. Antibiot., 49(8): 815-816 (1996), and Bioorg. Med. Chem. Lett., 6:1951-1954 (1996).

Squalene synthetase inhibitors useful in the present invention include, but are not limited to, those diclosed in Curr. Op. Ther. Patents, 8614, (1993); EP Patent Application Nos. 0 567 026 A1; 0 645 378 A1; 0 645 377 A1; 0 611 749 A1; 0 705 607 A2; 0 701 725 Al; and PCT publication WO 96/09827.

Anti-oxidants useful in the present invention include, but are not limited to, probucol; and pharmaceutically acceptable salts and esters thereof.

PPARα agonists useful in the present invention include, but are not limited to, beclofibrate, benzafibrate, ciprofibrate, clofibrate, etofibrate, fenofibrate, gemcabene, and gemfibrozil, GW 7647, BM 170744, LY518674; and other fibric acid derivatives, such as Atromid®, Lopid® and Tricor®, and the like.

FXR receptor antagonist useful in the present invention include, but are not limited to, GW 4064, SR 103912, and the like;

LXR receptor agonists useful in the present invention include, but are not limited to GW 3965, T9013137, and XTC0179628, and the like.

Lipoprotein synthesis inhibitors useful in the present invention include, but are not limited to, niacin or nicotinic acid.

Bile acid reuptake inhibitors useful in the present invention include, but are not limited to, BARI 1453, SC435, PHA384640, S8921, AZD7706, and the like.

PPARδ agonists useful in the present invention include, but are not limited to, GW 501516 and GW 590735 and the like, and those disclosed in WO97/28149.

Microsomal triglyceride transport inhibitors useful in the present invention include, but are not limited to, inplitapide, LAB687, and CP346086, and the like.

Any compound that decreases HMG-CoA reductase gene expression may be used as an additional compound in the combination therapy aspect of this invention. These agents may be HMG-CoA reductase transcription inhibitors that block the transcription of DNA or translation inhibitors that prevent translation of mRNA coding for HMG-CoA reductase into protein. Such inhibitors may either affect transcription or translation directly, or may be biotransformed into compounds that have the aforementioned attributes by one or more enzymes in the cholesterol biosynthetic cascade or may lead to the accumulation of an isoprene metabolite that has the aforementioned activities. Such regulation is readily determined by those skilled in the art according to standard assays (Methods of Enzymology, 110: 9-19 1985). Several such compounds are described and referenced below however other inhibitors of HMG-CoA reductase gene expression will be known to those skilled in the art. U.S. Pat. No. 5,041,432 discloses certain 15-substituted lanosterol derivatives. Other oxygenated sterols that suppress the biosynthesis of HMG-CoA reductase are discussed by E. I. Mercer (Prog. Lip. Res., 32:357-416 1993).

The above compounds are only illustrative of the anti-obesity agents and anti-dyslipidemic agents that can be used in the compositions of the present invention. As this listing of compounds is not meant to be comprehensive, the methods of the present invention may employ any anti-obesity agent and any anti-dyslipidemic agent, and are not limited to any particular structural class of compounds.

The present invention further relates to the treatment of obesity with a combination of an anti-obesity agent and an antidyslipidemic agent, which may be administered separately, therefore the invention also relates to combining separate pharmaceutical compositions into a kit form. The kit, according to this invention, comprises two separate pharmaceutical compositions: a first unit dosage form comprising a prophylactically or therapeutically effective amount of the anti-obesity agent, or a pharmaceutically acceptable salt or ester thereof, and a pharmaceutically acceptable carrier or diluent in a first unit dosage form, and a second unit dosage form comprising a prophylactically or therapeutically effective amount of the anti-dyslipidemic agent, or a pharmaceutically acceptable salt or ester thereof, and a pharmaceutically acceptable carrier or diluent in a second unit dosage form.

The present invention also relates to a kit comprising at least one unit dosage of a prophylactically or therapeutically effective amount of an anti-obesity agent, and pharmaceutically acceptable salts and esters thereof, and at least one unit dosage of a prophylactically or therapeutically effective amount of an anti-dyslipidemic agent, and pharmaceutically acceptable salts and esters thereof.

In one embodiment of the present invention, the kit further comprises a container. Such kits are especially suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered.

Combination Therapy

The compositions of the present invention may be used in combination with other drugs that may also be useful in the treatment, prevention, or control of those disorders, including dyslipidemia, dyslipidemia associated with obesity, dyslipidemia-related disorders, metabolic syndrome, cardiac hypertrophy, left ventricular hypertrophy, obesity and obesity-related disorders, for which compounds comprising the compositions are useful. Such other drugs may be administered, by a route and in an amount commonly used therefore, contemporaneously or sequentially with a composition of the present invention. When a composition of the present invention is used contemporaneously with one or more other drugs, a pharmaceutical composition in unit dosage form containing such other drugs and the composition of the present invention is preferred. However, the combination therapy also includes therapies in which the composition of the present invention and one or more other drugs are administered on different overlapping schedules. It is also contemplated that when used in combination with one or more other active ingredients, the composition of the present invention and the other active ingredients may be used in lower doses than when each is used singly. Accordingly, the pharmaceutical compositions of the present invention include those that contain one or more other active ingredients, in addition to a composition of the present invention.

Examples of other active ingredients that may be administered in combination with a composition of the present invention, and either administered separately or in the same pharmaceutical composition, include, but are not limited to:

(a) anti-diabetic agents such as (i) PPARγ agonists such as glitazones (e.g. ciglitazone; darglitazone; englitazone; isaglitazone (MCC-555); pioglitazone; rosiglitazone; troglitazone; CLX-0921; 5-BTZD, and the like), and GW-0207, LG-100641, and LY-300512, and the like; (ii) biguanides such as buformin; metformin; and phenformin, and the like; (iii) protein tyrosine phosphatase-1B (PTP-1B) inhibitors; (iv) sulfonylureas such as acetohexamide; chlorpropamide; diabinese; glibenclamide; glipizide; glyburide; glimepiride; gliclazide; glipentide; gliquidone; glisolamide; tolazamide; and tolbutamide, and the like; (v) meglitinides such as repaglinide, and nateglinide, and the like; (vi) alpha glucoside hydrolase inhibitors such as acarbose; adiposine; camiglibose; emiglitate; rniglitol; voglibose; pradimicin-Q; salbostatin; CKD-711; MDL-25,637; MDL-73,945; and MOR 14, and the like; (vii) alpha-amylase inhibitors such as tendamistat, trestatin, and A1-3688, and the like; (viii) insulin secreatagogues such as linogliride; and A4166, and the like; (ix) fatty acid oxidation inhibitors, such as clomoxir, and etomoxir, and the like; (x) A2 antagonists, such as midaglizole; isaglidole; deriglidole; idazoxan; earoxan; and fluparoxan, and the like; (xi) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH₂), and the like; (xii) non-thiazolidinediones such as JT-501, and farglitazar (GW-2570/GI-262579), and the like; (xiii) PPARα/γ dual agonists such as CLX-0940, GW-1536, GW1929, GW-2433, KRP-297, L-796449, LR-90, MK-0767, and SB 219994, and muraglitazar, and the like; (xiv) other insulin sensitizing drugs; (xv) a glucokinase activator; and (xvi) VPAC2 receptor agonists; and

(b) anti-hypertensive agents such as (i) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (ii) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (iii) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemiildipine, lercanidipine, nicardipine, nifedipine, nilvadipine, nimodepine, nisoldipine, nitrendipine, manidipine, pranidipine, and verapamil, and the like; (iv) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (v) neutral endopeptidase inhibitors such as omapatrilat, cadoxatril and ecadotril, fosidotril, sampatrilat, AVE7688, ER4030, and the like; (vi) endothelin antagonists such as tezosentan, A308165, and YM62899, and the like; (vii) vasodilators such as hydralazine, clonidine, minoxidil, and nicotinyl alcohol, and the like; (viii) angiotensin II receptor antagonists such as candesartan, eprosartan, irbesartan, losartan, pratosartan, tasosartan, telmisartan, valsartan, and EXP-3137, FI6828K, and RNH6270, and the like; (viv) α/β adrenergic blockers as nipradilol, arotinolol and amosulalol, and the like; (x) alpha 1 blockers, such as terazosin, urapidil, prazosin, bunazosin, trimazosin, doxazosin, naftopidil, indoramin, WHIP 164, and XEN010, and the like; and (xi) alpha 2 agonists such as lofexidine, tiamenidine, moxonidine, rilmenidine and guanobenz, and the like. Combinations of anti-obesity agents and diuretics or beta blockers may further include vasodilators, which widen blood vessels. Representative vasodilators useful in the compositions and methods of the present invention include, but are not limited to, hydralazine (apresoline), clonidine (catapres), minoxidil (loniten), and nicotinyl alcohol (roniacol).

The above combinations include combinations of a composition of the present invention not only with one other active compound, but also with two or more other active compounds. Non-limiting examples include combinations of the compositions of the present invention with one, two or more active compounds selected from anti-diabetic agents, and anti-hypertensive agents. Combinations of the compositions of the present invention with one, two or more active compounds selected from anti-diabetic agents, and anti-hypertensive agents will be useful to treat, control or prevent metabolic syndrome. Combinations of the compositions of the present invention comprising an anti-obesity agent, an anti-dyslipidemic agent, in addition to an anti-diabetic agent and/or an anti-hypertensive agent will be more efficacious in the treatment, control, or prevention of metabolic syndrome than treatment with any of these agents alone. In particular, compositions comprising an anti-obesity agent, an anti-dyslipidemic agent, an anti-hypertensive agent and/or an anti-diabetic agent will be useful to synergistically treat, control or prevent metabolic syndrome.

### DEFINITIONS

“Halogen atom” refers to fluorine atom, chlorine atom, bromine atom and iodine atom.

“Lower alkyl” refers to a straight- or branched-chain alkyl group of C₁ to C₆, for example, methyl, ethyl, propyl, isopropyl, butyl, isobutyl, sec-butyl, tert-butyl, pentyl, isopentyl, hexyl, isohexyl, and the like.

“Halo(lower)alkyl” refers to the aforesaid lower alkyl substituted with 1 or more than 2, preferably 1 to 3 aforesaid halogen atoms identically or differently at the substitutable, arbitrary positions, for example, fluoromethyl, difluoromethyl, trifluoromethyl, 2-fluoroethyl, 1,2-difluoroethyl, chloromethyl, 2-chloroethyl, 1,2-dichloroethyl, bromomethyl, iodomethyl, and the like.

“Hydroxy(lower)alkyl” refers to the aforesaid lower alkyl substituted with 1 or more than 2, preferably 1 or 2 hydroxy groups at the substitutable, arbitrary positions, for example, hydroxymethyl, 2-hydroxyethyl, 1-hydroxy-1-methylethyl, 1,2-dihydroxyethyl, 3-hydroxypropyl, and the like.

“Cyclo(lower)alkyl” refers to a cycloalkyl group of C₃ to C₆, for example, cyclopropyl, cyclobutyl, cyclopentyl, cyclohexyl, and the like.

“Lower alkenyl” refers to a straight- or branched-chain alkenyl group of C₂ to C₆, for example, vinyl, 1-propenyl, 2-propenyl, isopropenyl, 3-butenyl, 2-butenyl, 1-butenyl, 1-methyl-2-propenyl, 1-methyl-1-propenyl, 1-ethyl-1-ethenyl, 2-methyl-2-propenyl, 2-methyl-1-propenyl, 3-methyl-2-butenyl, 4-pentenyl, and the like.

“Lower alkoxy” refers to a straight- or branched-chain alkoxy group of C₁ to C₆, for example, methoxy, ethoxy, propoxy, isopropoxy, butoxy, sec-butoxy, isobutoxy, tert-butoxy, pentyloxy, isopentyloxy, hexyloxy, isohexyloxy, and the like.

“Halo(lower)alkoxy” refers to the aforesaid lower alkoxy substituted with 1 or more than 2, preferably 1 to 3 aforesaid halogen atoms identically or differently at the substitutable, arbitrary positions, for example, fluoromethoxy, difluoromethoxy, trifluoromethoxy, 2-fluoroethoxy, 1,2-difluoroethoxy, chloromethoxy, 2chloroethoxy, 1,2-dichloroethoxy, bromomethoxy, iodomethoxy, and the like.

“Lower alkylthio” refers to a straight- or branched-chain alkylthio group of C₁ to C₆, for example, methylthio, ethylthio, propylthio, isopropylthio, butylthio, sec-butylthio, isobutylthio, tert-butylthio, pentylthio, isopentylthio, hexylthio, isohexylthio, and the like.

“Lower alkanoyl” refers to an alkanoyl group containing the aforesaid lower alkyl, that is, an alkanoyl group of C₂ to C₇, for example acetyl, propionyl, butyryl, isobutyryl, valeryl, isovaleryl, pivaloyl, and the like.

“Lower alkoxycarbonyl” refers to an alkoxycarbonyl group containing the aforesaid lower alkoxy, that is, an alkoxycarbonyl group of C₂ to C₇, for example, methoxycarbonyl, ethoxycarbonyl, propoxycarbonyl, isopropoxycarbonyl, butoxycarbonyl, isobutoxycarbonyl, tert-butoxycarbonyl, pentyloxycarbonyl, and the like.

“Lower alkylene optionally substituted with oxo” refers to a straight- or branched-chain alkylene group of C₂ to C₆ which may be substituted with 1 or more than 2, preferably 1 oxo group at a substitutable, arbitrary position, for example, ethylene, trimethylene, tetramethylene, pentamethylene, hexamethylene, 1-oxoethylene, 1-oxotrimethylene, 2-oxotrimethylene, 1-oxotetramethylene, 2-oxotetramethylene, and the like.

“Aryl” includes phenyl, naphthyl, and the like.

“Heteroaryl” refers to 5- or 6-membered monocylic heteroaromatic group which contains 1 or more than 2, preferably 1 to 3 hetero atoms identically or differently selected from the group of oxygen atom, nitrogen atom and sulfur atom; or condensed heteroaromatic group, where the aforesaid monocylic heteroaromatic group is condensed with the aforesaid aryl group, or with the identified or different aforesaid monocylic heteroaromatic group each other, for example, pyrrolyl, furyl, thienyl, imidazolyl, pyrazolyl, thiazolyl, isothiazolyl, oxazolyl, isoxazolyl, 1,2,3-triazolyl, 1,2,4-triazolyl, tetrazolyl, oxadiazolyl, 1,2,3-thiadiazolyl, 1,2,4-thiadiazolyl, 1,3,4-thiadiazolyl, pyridyl, pyrazinyl, pyrimidinyl, pyridazinyl, 1,2,4-triazinyl, 1,3,5-triazinyl, indolyl, benzofuranyl, benzothienyl, benzimidazolyl, benzoxazolyl, benzisoxazolyl, benzothiazolyl, benzisothiazolyl, indazolyl, purinyl, quinolyl, isoquinolyl, phthalazyl, naphthylidinyl, quinoxalinyl, quinazolinyl, cinnolinyl, pteridinyl, pyrido[3,2-b]pyridyl, and the like.

“Lower alkylamino” refers to an amino group mono-substituted with the aforesaid lower alkyl, for example, methyl amino, ethylamino, propylamino, isopropylamino, butylamino, sec-butylamino, tert-butylamino, and the like.

“Di-lower alkylamino” refers to an amino group di-substituted with identical or different aforesaid lower alkyl, for example, dimethylamino, diethylamino, ethylmethylamino, dipropylamino, methylpropylamino, diisopropylamino, and the like.

In order to disclose the aforesaid compounds of the general formula (I) more detailed, the various symbols used in the formula (I) are explained in more detail by the use of preferred embodiments.

Ar¹ represents aryl or heteroaryl which may be substituted, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-Ar².

“Aryl or heteroaryl which may be substituted, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-Ar²” refers to unsubstituted aforesaid aryl or aforesaid heteroaryl, or the aforesaid aryl or aforesaid heteroaryl which has substituent(s) at the substitutable, arbitrary position(s). The aforesaid substituent can be, identically or differently, one or more than 2, preferably 1 or 2 selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group of formula: -Q-Ar².

Halogen atom as the aforesaid substituent includes fluorine atom, chlorine atom, and the like preferably.

Lower alkyl as the aforesaid substituent includes methyl, ethyl, propyl, isopropyl, and the like preferably.

Halo(lower)alkyl as the aforesaid substituent includes difluoromethyl, trifluoromethyl, and the like preferably.

Hydroxy(lower)alkyl as the aforesaid substituent includes hydroxymethyl, 2-hydroxyethyl, 1-hydroxy-1-methylethyl, and the like preferably.

Cyclo(lower)alkyl as the aforesaid substituent includes cyclopropyl, cyclobutyl, and the like preferably.

Lower alkenyl as the aforesaid substituent includes vinyl, 1-propenyl, 2-methyl-1-propenyl, and the like preferably.

Lower alkoxy as the aforesaid substituent includes methoxy, ethoxy, and the like preferably.

Halo(lower)alkoxy as the aforesaid substituents includes fluoromethoxy, difluoromethoxy, trifluoromethoxy, and the like preferably.

Lower alkylthio as the aforesaid substituent includes methylthio, ethylthio, and the like preferably.

Lower alkanoyl as the aforesaid substituent includes acetyl, propionyl, and the like preferably.

Lower alkoxycarbonyl as the aforesaid substituent includes methoxycarbonyl, ethoxycarbonyl, and the like preferably.

Lower alkylene optionally substituted with oxo as the aforesaid substituent includes 1-oxotetramethylene, and the like preferably.

In a group of formula: -Q-Ar² as the aforesaid substituent, Ar² represents aryl or heteroaryl which may be substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;

Q represents a single bond or carbonyl.

“Aryl or heteroaryl which may be substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl” refers to unsubstituted aforesaid aryl or aforesaid heteroaryl, or the aforesaid aryl or aforesaid heteroaryl which has substituent(s) at the substitutable, arbitrary position(s). The aforesaid substituent can be, identically or differently, one or not less than 2, preferably 1 or 2 selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl.

Halogen atom as the aforesaid substituent includes, preferably, fluorine atom, chlorine atom, and the like.

Lower alkyl as the aforesaid substituent includes, preferably, methyl, ethyl, propyl, isopropyl, and the like.

Halo(lower)alkyl as the aforesaid substituent includes, preferably, difluoromethyl, trifluoromethyl, and the like.

Hydroxy(lower)alkyl as the aforesaid substituent includes, preferably, hydroxymethyl, 2-hydroxyethyl, 1-hydroxy-1-methylethyl, and the like.

Lower alkoxy as the aforesaid substituent includes, preferably, methoxy, ethoxy, and the like.

Halo(lower)alkoxy as the aforesaid substituent includes, preferably, fluoromethoxy, difluoromethoxy, trifluoromethoxy, and the like.

Lower alkylamino as the aforesaid substituent includes, preferably, methylamino, ethylamino, and the like.

Di-lower alkylamino as the aforesaid substituent includes, preferably, dimethylamino, diethylamino, and the like.

Lower alkanoyl as the aforesaid substituent includes, preferably, acetyl, propionyl, and the like.

Aryl as the aforesaid substituent includes, preferably, phenyl, and the like.

The substituent(s) of Ar² include, preferably, halogen, cyano, lower alkyl, halo(lower)alkyl, hydroxy(lower)alkyl, hydroxy, halo(lower)alkoxy, and the like.

Aryl in Ar² includes, preferably, phenyl, and the like and heteroaryl includes imidazolyl, pyridyl, benzofuranyl, quinolyl, and the like.

Consequently, a group of formula: -Q-Ar² includes, for example, phenyl, 2-fluorophenyl, 3-fluorophenyl, 4-fluorophenyl, 2,3-difluorophenyl, 2,4-difluorophenyl, 3,5-difluorophenyl, 2-chlorophenyl, 3-chlorophenyl, 4-chlorophenyl, 2-cyanophenyl, 3-cyanophenyl, 4-cyanophenyl, 2-methylphenyl, 3-methylphenyl, 4-methylphenyl, 2-fluoro-5-methylphenyl, 3-fluoromethylphenyl, 2-trifluoromethylphenyl, 3-trifluoromethylphenyl, 4-trifluoromethylphenyl, 2-methoxyphenyl, 3-methoxyphenyl, 4-methoxyphenyl, 3-fluoro-5-methoxyphenyl, 3-fluoromethoxyphenyl, 3-difluoromethoxyphenyl, 3-(2-hydroxyethyl)phenyl, 3-hydroxymethylphenyl, 3-(1-hydroxy-1-methylethyl)phenyl, 3-hydroxyphenyl, 4-hydroxyphenyl, 2-imidazolyl, 1-ethyl-2-imidazolyl, 1,2,4-thiadiazol-5-yl, 1,3,4-thiadiaol-2-yl, 2-pyridyl, 3-pyridyl, 4-pyridyl, 2-ethyl4-pyridyl, 4-pyrimidinyl, 5-pyrimidinyl, 4-benzo[b]furanyl, 5-benzo[b]furanyl, 7-benzo[b]furanyl, 2-quinolyl, 3-quinolyl, 4-quinolyl, 5-quinolyl, 6-quinolyl, 8-quinolyl, benzoyl, 2-pyridylcarbonyl, and the like, and preferably, phenyl, 2-fluorophenyl, 3-fluorophenyl, 3,5-difluorophenyl, 3-chlorophenyl, 4-chlorophenyl, 3-cyanophenyl, 3-trifluoromethylphenyl, 3-difluoromethoxyphenyl, 3-(2-hydroxyethyl)phenyl, 3-hydroxyphenyl, 4-hydroxyphenyl, 1-ethyl-2-imidazolyl, 2-pyridyl, 7-benzo[b]furanyl, 2-quinolyl, 3-quinolyl, benzoyl, 2-pyridylcarbonyl, and the like.

The substituent of Ar¹ includes, preferably, halogen, lower alkyl, halo(lower)alkyl, lower alkenyl, lower alkanoyl, lower alkylene optionally substituted with oxo, and a group of formula: -Q-Ar², and the like.

Aryl in Ar¹ includes, preferably, phenyl, and the like and heteroaryl of Ar¹ includes pyrrolyl, imidazolyl, pyrazolyl, thiazolyl, oxazolyl, isoxazolyl, 1,2,3-triazolyl, 1,2,4-thiadiazolyl, pyridyl, pyrazinyl, pyrimidinyl, 1,2,4-triazinyl, benzoxazolyl, benzothiazolyl, quinolyl, pyrido[3,2-b]pyridyl, and the like.

Consequently, Ar¹ includes, for example, 3-fluorophenyl, 4-fluorophenyl, 3,4-difluorophenyl, 3-chlorophenyl, 4-chlorophenyl, 3,4-dichlorophenyl, 4-acetylphenyl, 5-oxo-5,6,7,8-tetrahydro-2-naphthyl, 4-acetyl-3-trifluoromethylphenyl, 4-(1-ethyl-2-imidazolyl)phenyl, 3-(2-pyridyl)phenyl, 3-(4-pyridyl)phenyl, 4-(2-pyridyl)phenyl, 4-(3-pyridyl)phenyl, 4-(2-ethyl-4-pyridyl)phenyl, 4-(4-pyrimidinyl)phenyl, 4-benzoylphenyl, 4-(2-pyridylcarbonyl)phenyl, 1-phenyl-3-pyrrolyl, 1-phenyl-4-imidazolyl, 1-(2-fluorophenyl)-4-imidazolyl, 1-(3-fluorophenyl)-4-imidazolyl, I-(4-fluorophenyl)-4-imidazolyl, 1-(2,3-difluorophenyl)-4-imidazolyl, 1-(2,4-difluorophenyl)-4-imidazolyl, 1-(3,5-difluorophenyl)-4-imidazolyl, 1-(3-chlorophenyl)-4-imidazolyl, 1-(2-cyanophenyl)-4-imidazolyl, 1-(3-cyanophenyl)-4-imidazolyl, 1-4-cyanophenyl)-4-imidazolyl, 1-(3-trifluoromethylphenyl)-4-imidazolyl, 1-[3-(2-hydroxyethyl)phenyl]-4-imidazolyl, 1-[3-(1-hydroxy-1-methylethyl)phenyl]-4-imidazolyl, 1-(3-methoxyphenyl)-4-imidazolyl, 1-(2-difluoromethoxyphenyl)-4-imidazolyl, 1-(3-difluoromethoxyphenyl)-4-imidazolyl, 1-(4-difluoromethoxy-phenyl)4-imidazolyl, 1-(2-pyridyl)-4-imidazolyl, 1-(4-benzo[b]furanyl)-4-imidazolyl, 1-(5-benzo[b]furanyl)-4-imidazolyl, 1-(7-benzo[b]furanyl)-4-imidazolyl, 1-(2-quinolyl)-4-imidazolyl, 1-(3-quinolyl)-4-imidazolyl, 1-(4-quinolyl)-4-imidazolyl, 1-(5-quinolyl)-4-imidazolyl, 1-(6-quinolyl)-4-imidazolyl, 1-(8-quinolyl)-4-imidazolyl, 1-phenyl-3-pyrazolyl, 5-phenyl-3-pyrazolyl, 1-phenyl-4-pyrazolyl, 1-(2-fluorophenyl)-3-pyrazolyl, 5-(2-fluorophenyl)-3-pyrazolyl, 5-(3-fluorophenyl)-3-pyrazolyl, 1-(3-fluorophenyl)-4-pyrazolyl, 1-(4-fluorophenyl)-3-pyrazolyl, 5-(4-fluorophenyl)-3-pyrazolyl, 5-(2-chlorophenyl)-3-pyrazolyl, 5-(3-chlorophenyl)-3-pyrazolyl, 5-(4-chlorophenyl)-3-pyrazolyl, 5-(2-difluoromethoxyphenyl)-3-pyrazolyl, 5-(3-difluoromethoxyphenyl)-3-pyrazolyl, 2-methyl-5-phenyl-3-pyrazolyl, 5-(2-pyridyl)-3-pyrazolyl, 5-(2-quinolyl)-3-pyrazolyl, 5-(3-quinolyl)-3-pyrazolyl, 4-phenyl-2-thiazolyl, 5-phenyl-2-thiazolyl, 5-(3-chlorophenyl)-2-thiazolyl, 5-(4-chlorophenyl)-2-thiazolyl, 5-(4-methoxyphenyl)-2-thiazolyl, 5-(2-pyridyl)-2-thiazolyl, 2-phenyl-4-thiazolyl, 4-phenyl-2-oxazolyl, 5-phenyl-2-oxazolyl, 4-(2-fluoromethoxyphenyl)-2-oxazolyl, 4-(3-fluoromethoxyphenyl)-2-oxazolyl, 5-phenyl-3-isoxazolyl, 3-phenyl-5-isoxazolyl, 3-(2-chlorophenyl)-5-isoxazolyl, 3-(3-chlorophenyl)-5-isoxazolyl, 3-(4-chlorophenyl)-5-isoxazolyl, 3-(2-pyridyl)-5-isoxazolyl, 2-phenyl-1,2,3-triazol-4-yl, 5-phenyl-1,2,4-thiadiazol-3-yl, 5-phenyl-1,3,4-thiadiazol-2-yl, 5-(3-chlorophenyl)-1,3,4-thiadiazol-2-yl, 5-(2-pyridyl)-1,3,4-thiadiazol-2-yl, 5-(2-ethyl-4-pyridyl)-1,3,4-thiadiazol-2-yl, 5-phenyl-2-pyridyl, 6-phenyl-3-pyridyl, 2-phenyl-4-pyridyl, 5-(2-pyridyl)-2-pyridinyl, 5-benzoyl-2-pyridyl, 6-benzoyl-3-pyridyl, 5-chloro-2-pyrazinyl, 5-(2-methyl-1-propenyl)-2-pyrazinyl, 5-acetyl-2-pyrazinyl, 5-propionyl-2-pyrazinyl, 5-phenyl-2-pyrazinyl, 5-(3-hydroxyphenyl)-2-pyrazinyl, 5-(4-hydroxyphenyl)-2-pyrazinyl, 5-(1,2,4-thiadiazol-5-yl)-2-pyrazinyl, 5-(1,3,4-thiadiazol-2yl)-2-pyrazinyl, 5-(2-pyridyl)-2-pyrazinyl, 5-(3-pyridyl)-2-pyrazinyl, 5-(5-pyrimidinyl)-2-pyrazinyl, 5-(3-quinolyl)-2-pyrazinyl, 5-benzoyl-2-pyrazinyl, 5-(2-pyridylcarbonyl)-2-pyrazinyl, 5-acetyl-2-pyrimidinyl, 5-acetyl-3-methyl-2-pyrimidinyl, 4-phenyl-2-pyrimidinyl, 5-phenyl-2-pyrimidinyl, 6-phenyl-4-pyrimidinyl, 2-phenyl-5-pyrimidinyl, 5-(2-fluorophenyl)-2-pyrimidinyl, 5-(3-fluorophenyl)-2-pyrimidinyl, 5-(4-fluorophenyl)-2-pyrimidinyl, 5-(2-chlorophenyl)-2-pyrimidinyl, 5-(3-chlorophenyl)-2-pyrimidinyl, 5-(4-chlorophenyl)-2-pyrimidinyl, 5-(2-methylphenyl)-2-pyrimidinyl, 5-(3-methylphenyl)-2-pyrimidinyl, 5-(2-fluoromethylphenyl)-2-pyrimidinyl, 5-(3-fluoromethylphenyl)-2-pyrimidinyl, 5-(2-trifluoromethylphenyl)-2-pyrimidinyl, 5-(3-trifluoromethylphenyl)-2-pyrimidinyl, 5-(4-trifluoromethylphenyl)-2-pyrimidinyl, 5-(2-hydroxymethylphenyl)-2-pyrimidinyl, 5-(3-hydroxymethylphenyl)-2-pyrimidinyl, 5-(2-hydroxyphenyl)-2-pyrimidinyl, 5-(3-hydroxyphenyl)-2-pyrimidinyl, 5-(2-methoxyphenyl)-2-pyrimidinyl, 5-(3-methoxyphenyl)-2-pyrimidinyl, 5-(4-methoxyphenyl)-2-pyrimidinyl, 5-(2-fluoromethoxyphenyl)-2-pyrimidinyl, 5-(3-fluoromethoxyphenyl)-2-pyrimidinyl, 5-(2-fluoro-5-methylphenyl)-2-pyrimidinyl, 5-(3-fluoro-5-methoxyphenyl)-2-pyrimidinyl, 6-phenyl-3-pyridazinyl, 6-phenyl-1,2,4-triazin-3-yl, 5-chloro-2-benzoxazolyl, 5-fluoro-2-benzothiazolyl, 4-methyl-2-benzothiazolyl, 2-methyl-5-benzothiazolyl, 4-methoxy-2-benzothiazolyl, 3-quinolyl, 6-quinolyl, 7-methyl-2-quinolyl, 2-methyl-6-quinolyl, 6-chloro-2-quinoxalinyl, pyrido[3,2-b]pyridin-2-yl, 7-chloropyrido[3,2-b]pyridin-2-yl, 7-methylpyrido[3,2-b]pyridin-2-yl, 7-trifluoromethylpyrido[3,2-b]pyridin-2-yl, 7-difluoromethoxypyrido[3,2-b]pyridin-2-yl, 7-acetylpyrido[3,2-b]pyridin-2-yl, and the like, preferably 3,4-dichlorophenyl, 4-acetylphenyl, 5-oxo-5,6,7,8-tetrahydro-2-naphthyl, 4-acetyl-3-trifluoromethylphenyl, 4-(1-ethyl-2-imidazolyl)phenyl, 4-benzoylphenyl, 4-(2-pyridylcarbonyl)phenyl, 1-phenyl-3-pyrrolyl, 1-phenyl-4-imidazolyl, 1-(2-fluorophenyl)-4-imidazolyl, 1-(3,5-difluorophenyl)-4-imidazolyl, 1-(3-chlorophenyl)-4-imidazolyl, 1-(3-cyanophenyl)-4-imidazolyl, 1-[3-(2-hydroxyethyl)phenyl]-4-imidazolyl, 1-(3-difluoromethoxyphenyl)-4-imidazolyl, 1-(7-benzo[b]furanyl)-4-imidazolyl, 1-(2-quinolyl)-4-imidazolyl, 1-(3-quinolyl)4-imidazolyl, 1-phenyl-3-pyrazolyl, 5-phenyl-3-pyrazolyl, 1-phenyl-4-pyrazolyl, 1-(3-fluorophenyl)-4-pyrazolyl, 1-(4-fluorophenyl)-3-pyrazolyl, 5-(4-chlorophenyl)-3-pyrazolyl, 5-(3-quinolyl)-3-pyrazolyl, 5-phenyl-2-thiazolyl, 3-phenyl-5-isoxazo methyl-1-propenyl)-2-pyrazinyl, 5-phenyl-2-pyrazinyl, 5-(3-hydroxyphenyl)-2-pyrazinyl, 5-(4-hydroxyphenyl)-2-pyrazinyl, 5-(2-pyridyl)-2-pyrazinyl, 5-benzoyl-2-pyrazinyl, 5-phenyl-2-pyrimidinyl, 5-(2-fluorophenyl)-2-pyrimidinyl, 5-3-fluorophenyl)-2-pyrimidinyl, 5-(3-chlorophenyl)-2-pyrimidinyl, 5-(3-trifluoromethyl-phenyl)-2-pyrimidinyl, 5-chloro-2-benzoxazolyl, 4-methyl-2-benzothia-zolyl, 7-methyl-2-quinolyl, 7-trifluoromethylpyrido[3,2-b]pyridin-2-yl, and the like, especially 1-phenyl-3-pyrazolyl, 5-phenyl-3-pyrazolyl, 5-phenyl-2-pyrazinyl, 5-(3-hydroxyphenyl)-2-pyrazinyl, 5-(4-hydroxyphenyl)-2-pyrazinyl, 5-phenyl-2-pyrimidinyl, 5-(2-fluorophenyl)-2-pyrimidinyl, 5-(3-fluorophenyl)-2-pyrimidinyl, 7-trifluoro-methylpyrido[3,2-b]pyridin-2-yl, and the like.

n represents 0 or 1, 0 is preferable.

T, U, V and W represent independently nitrogen atom or methine which may have a substituent selected from the group consisting of halogen, lower alkyl, hydroxy and lower alkoxy, where at least two of them represent the said methine group.

“Methine which may have a substituent selected from the group consisting of halogen, lower alkyl, hydroxy and lower alkoxy” refers to unsubstituted methine or methine having a substituent which can be selected from the group consisting of halogen, lower alkyl, hydroxy and lower alkoxy.

Halogen atom as the aforesaid substituent includes preferably fluorine atom, chlorine atom, and the like.

Lower alkyl as the aforesaid substituent includes preferably methyl, ethyl, and the like.

Lower alkoxy as the aforesaid substituent includes preferably methoxy, ethoxy, and the like.

The aforesaid substituent include preferably halogen, and the like.

The preferred mode of T, U, V and W includes, for example, T, U, V and W are independently methine optionally having the aforesaid substituent, preferably halogen; or one of T, U, V and W is nitrogen atom.

X represents methine or nitrogen.

Y represents imino which may be substituted with lower alkyl, or oxygen.

“imino which may be substituted with lower alkyl” refers to unsubstituted imino or imino substituted with lower alkyl.

The aforesaid lower alkyl includes, preferably, methyl, ethyl, and the like.

Y is preferably unsubstituted imino or oxygen, especially oxygen.

The term “pharmaceutically acceptable salts” refers to the pharmaceutically acceptable and common salts, for example, a base addition salt to carboxyl group when the compound has a carboxyl group, or an acid addition salt to amino or basic heterocyclyl when the compound has an amino or basic heterocyclyl group, including quaternary ammonium salts, prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, trifluoro acetate, isothionate, triethiodide, lactate, panoate, valerate, and the like which can be used as a dosage form for modifying the solubility or hydrolysis characteristics or can be used in sustained release or pro-drug formulations.

It will be understood that, as used herein, references to anti-obesity agents, and to anti-dyslipidemic agents are meant to also include the pharmaceutically acceptable salts and esters thereof.

The term “at least one anti-obesity agent” includes within its meaning a single anti-obesity agent or two anti-obesity agents, each of which may be selected from a distinct class of anti-obesity agents, e.g. an NPY5 antagonist with a lipase inhibitor. The term “at least one anti-dyslipidemic agent” includes within its meaning a single anti-dyslipidemic agent or two anti-dyslipidemic agents, each of which may be selected from a distinct class of anti-dyslipidemic agents, e.g. a HMG-CoA reductase inhibitor with a cholesterol absorption inhibitor.

The pharmaceutically acceptable salts of the composition of the instant invention include the composition wherein one of the individual components of the composition is in the form of a pharmaceutically acceptable salt, or the composition wherein all of the individual components are in the form of pharmaceutically acceptable salts (wherein the salts for each of the components can be the same or different), or a pharmaceutically acceptable salt of the combined components (i.e., a salt of the composition).

The “pharmaceutically acceptable esters” in the present invention refer to non-toxic esters, for example, the pharmaceutically acceptable, common esters on carboxyl group when the compound has a carboxyl group, for example, esters with lower alkyls (for example methyl, ethyl, propyl, isopropyl, butyl, sec-butyl, tert-butyl, pentyl, isopentyl, neopentyl, cyclopropyl, cyclobutyl, cyclopentyl), aralkyls (for example benzyl, phenethyl), lower alkenyls (for example allyl, 2-butenyl), lower alkoxy (lower) alkyls (for example methoxymethyl, 2-methoxyethyl, 2-ethoxyethyl), lower alkanoyloxy (lower) alkyls (for example acetoxymethyl, pivaloyloxy-methyl, 1-pivaloyloxyethyl), lower alkoxycarbonyl (lower) alkyls (for example methoxycarbonylmethyl, isopropoxycarbonylmethyl), carboxy-(lower)alkyls (for example carboxymethyl), lower alkoxycarbonyloxy-(lower)alkyls (for example 1-(ethoxycarbonyloxy)ethyl, 1-(cyclohexyl-oxycarbonyloxy)ethyl), carbamoyloxy(lower)alkyls (for example carbamoyloxymethyl), phthalidyl group, (5-substituted-2-oxo-1,3-dioxol-4-yl)methyl (for example (5-methyl-2-oxo-1,3-dioxol-4-yl)methyl), and the like.

The compounds in the compositions of the present invention include stereoisomers, such as optical isomers, diastereomers and geometerical isomers, or tautomers depending on the mode of substitution. The compounds may contain one or more chiral centers and occur as racemates, racemic mixtures and as individual diastereomers, enantiomeric mixtures or single enantiomers, or tautomers, with all isomeric forms being included in the present invention. The present invention is meant to comprehend all such isomeric forms of the compounds in the compositions of the present invention, and their mixtures. Therefore, where a compound is chiral, the separate enantiomers, substantially free of the other, are included within the scope of the invention; further included are all mixtures of the two enantiomers. Also included within the scope of the invention are polymorphs, hydrates and solvates of the compounds of the instant invention.

The present invention includes within its scope prodrugs of the compounds in the compositions of this invention. In general, such prodrugs will be functional derivatives of the compounds in these compositions which are readily convertible in vivo into the required compound. Thus, in the methods of treatment of the present invention, the term “administering” shall encompass the treatment of obesity and obesity-related disorders with the compounds specifically disclosed as elements of the composition or with compounds which may not be specifically disclosed, but which convert to the specified compounds in vivo after administration to the patient. Conventional procedures for the selection and preparation of suitable prodrug derivatives are described, for example, in “Design of Prodrugs,” ed. H. Bundgaard, Elsevier, 1985.

Utilities

The compositions of the present invention are useful for the treatment, control or prevention of dyslipidemia. The dyslipidemia herein may be due to any cause, whether genetic or environmental. The compositions of the present invention are useful for the treatment, control or prevention of dyslipidemia associated with obesity. The term “dyslipidemia associated with obesity” refers to dyslipidemia caused by obesity or resulting from obesity.

Treatment of dyslipidemia, or a dyslipidemia related disorder, refers to the administration of the combinations of the present invention to a dyslipidemic subject. Prevention of dyslipidemia, or a dyslipidemia associated disorder, refers to the administration of the combinations of the present invention to a pre-dyslipidemic subject. A pre-dyslipidemic subject is a subject with higher than normal lipid levels that is not yet dyslipidemic.

The compositions of the present invention are useful for the treatment, control, or prevention of dyslipidemia-related disorders. The dyslipidemia-related disorders herein are associated with, caused by, or result from dyslipidemia. Examples of dyslipidemia-related disorders include lipid disorders, hyperlipidemia, hypertriglyceridemia, hypercholesterolemia, low high density lipoprotein (HDL) levels, high plasma low density lipoprotein (LDL) levels, atherosclerosis and its sequelae, coronary artery or carotid artery disease, heart attack, and stroke. In particular, dyslipidemia-related disorders include hyperlipidemia, hypertriglyceridemia, hypercholesterolemia, low high density lipoprotein (HDL) levels, high plasma low density lipoprotein (LDL) levels, and atherosclerosis. Dyslipidemia-related disorders further include metabolic syndrome.

The compositions of the present invention are useful for the treatment, control, or prevention of cardiac hypertrophy and/or left ventricular hypertrophy.

The compositions of the present invention are also useful for the treatment, control, or prevention of metabolic syndrome.

The compositions of the present invention are useful for the treatment, control, or prevention of obesity and obesity-related disorders. The obesity herein may be due to any cause, whether genetic or environmental.

The obesity-related disorders herein are associated with, caused by, or result from obesity. Examples of obesity-related disorders include diabetes, overeating, binge eating, and bulimia, hypertension, elevated plasma insulin concentrations and insulin resistance, dyslipidemias, hyperlipidemia, endometrial, breast, prostate, kidney and colon cancer, osteoarthritis, obstructive sleep apnea, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-sophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, hyperuricaemia, lower back pain, gallbladder disease, hyperuricemia, gout, and kidney cancer, and increased anesthetic risk. The compositions of the present invention are also useful to treat Alzheimer's disease.

The term “dyslipidemia” includes various disorders of lipid metabolism, such as conditions characterized by abnormal concentrations of one or more lipids (i.e. cholesterol and triglycerides), and/or apolipoproteins (i.e., apolipoproteins A, B, C and E), and/or lipoproteins (i.e., the macromolecular complexes formed by the lipid and the apolipoprotein that allow lipids to circulate in blood, such as LDL, VLDL and IDL). The term “dyslipidemia” includes hyperlipidemia, which is associated with abnormally high levels of lipids, LDL and VLDL cholesterol, and/or triglycerides.

The compositions of the present invention are expected to be efficacious in the treatment of lipid disorders, such as dyslipidemia, hyperlipidemia, hypercholesterolemia, hypertriglyceridemia, and low levels of high density lipoproteins; for lowering plasma low density lipoprotein and/or total cholesterol, for lowering plasma triglycerides, for preventing or reducing the risk of developing atherosclerosis, as well as for halting or slowing the progression of atherosclerotic disease once it has become clinically evident. The compositions of the present invention are also useful in the treatment, control and/or prevention of coronary artery or carotid artery disease, heart attack, and stroke.

Prevention of dyslipidemia refers to the administration of a compound or combination of the present invention to prevent the onset of dyslipidemia in a subject in need thereof.

The term “atherosclerosis” as used herein encompasses vascular diseases and conditions that are recognized and understood by physicians practicing in the relevant fields of medicine. Atherosclerotic cardiovascular disease, coronary heart disease (also known as coronary artery disease or ischemic heart disease), cerebrovascular disease and peripheral vessel disease are all clinical manifestations of atherosclerosis and are therefore encompassed by the terms “atherosclerosis” and “atherosclerotic disease.” The combination comprised of a therapeutically effective amount of an anti-obesity agent in combination with a therapeutically effective amount of an anti-dyslipidemic agent may be administered to prevent or reduce the risk of occurrence, or recurrence where the potential exists, of a coronary heart disease event, a cerebrovascular event, or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

The term “metabolic syndrome”, also known as syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following disorders: abdominal obesity, hypertriglyceridemia, low HDL cholesterol, high blood pressure, and high fasting plasma glucose. The criteria for these are defined in ATP-III. Treatment of metabolic syndrome refers to the administration of the combinations of the present invention to a subject with metabolic syndrome. Prevention of metabolic syndrome refers to the administration of the combinations of the present invention to a subject with two of the disorders that define metabolic syndrome. A subject with two of the disorders that define metabolic syndrome is a subject that has developed two of the disorders that define metabolic syndrome, but has not yet developed three or more of the disorders that define metabolic syndrome.

Left ventricular hypertrohpy (LVH) is identified based on left ventricular mass index (LVMI) and relative wall thickness (RWT). Left ventricular mass index is defined as left ventricular mass in grams divided by body surface area in meters². Relative wall thickness is defined as 2× posterior wall thickness/left ventricular end diastolic diameter. Normal LVMI values are typically 85 and normal RWT approximately 0.36. A male subject with LVH has a LVMI greater than 131 g/m²; a female subject with LVH has a LVMI greater than 100 g/m². A subject with an elevated LVMI value is a male subject with a LVMI between 85 g/m² and 131 g/m², or a female subject with a LVMI between 85 g/m² and 100 g/m².

Treatment of cardiac hypertrophy, or left ventricular hypertrophy, refers to the administration of the combinations of the present invention to a subject with cardiac hypertrophy or left ventricular hypertrophy. Prevention of cardiac hypertrophy, or left ventricular hypertrophy, refers to the administration of the combinations of the present invention to decrease or maintain the LVMI in a subject with an elevated LVMI value or to prevent the increase of LVMI in a subject with a normal LVMI value.

One outcome of treatment of cardiac hypertrophy or left ventricular hypertrophy may be a decrease in ventricular mass. Another outcome of treatment of cardiac hypertrophy or left ventricular hypertrophy may be a decrease in the rate of increase of ventricular mass. Another outcome of treatment of cardiac hypertrophy or left ventricular hypertrophy may be a decrease in ventricular wall thickness. Another outcome of treatment of cardiac hypertrophy of left ventricular hypertrophy may be the decrease in the rate of increase in ventricular wall thickness.

The term “obesity” as used herein is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus—type 2, diabetes associated with obesity, impaired glucose tolerance, impaired fasting glucose, insulin resistance syndrome, dyslipidemia, hypertension, hypertension associated with obesity, hyperuricemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

Treatment of obesity and obesity-related disorders refers to the administration of the compounds or combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the compounds or combinations of the present invention to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type 2 diabetes, polycystic ovary disease, cardiovascular diseases, osteoarthritis, dermatological disorders, hypertension, insulin resistance, hypercholesterolemia, hypertriglyceridemia, and cholelithiasis.

The term “diabetes,” as used herein, includes both insulin-dependent diabetes mellitus (i.e., IDDM, also known as type 1 diabetes) and non-insulin-dependent diabetes mellitus (i.e., NIDDM, also known as Type 2 diabetes). Type 1 diabetes, or insulin-dependent diabetes, is the result of an absolute deficiency of insulin, the hormone which regulates glucose utilization. Type 2 diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type 2 diabetics are also obese. The compositions of the present invention are useful for treating both Type 1 and Type 2 diabetes. The term “diabetes associated with obesity” refers to diabetes caused by obesity or resulting from obesity. The compositions are especially effective for treating Type 2 diabetes. The compositions of the present invention are also useful for treating and/or preventing gestational diabetes mellitus.

Diabetes is characterized by a fasting plasma glucose level of greater than or equal to 126 mg/dl. A diabetic subject has a fasting plasma glucose level of greater than or equal to 126 mg/dl. Prediabetes is characterized by an impaired fasting plasma glucose (FPG) level of greater than or equal to 110 mg/dl and less than 126 mg/dl; or impaired glucose tolerance; or insulin resistance. A prediabetic subject is a subject with impaired fasting glucose (a fasting plasma glucose (FPG) level of greater than or equal to 110 mg/dl and less than 126 mg/dl); or impaired glucose tolerance (a 2 hour plasma glucose level of ≧140 mg/dl and <200 mg/dl); or insulin resistance, resulting in an increased risk of developing diabetes.

Treatment of diabetes mellitus refers to the administration of a compound or combination of the present invention to treat a diabetic subject. One outcome of treatment may be decreasing the glucose level in a subject with elevated glucose levels. Another outcome of treatment may be decreasing insulin levels in a subject with elevated insulin levels. Another outcome of treatment may be decreasing plasma triglycerides in a subject with elevated plasma triglycerides. Another outcome of treatment is decreasing LDL cholesterol in a subject with high LDL cholesterol levels. Another outcome of treatment may be increasing HDL cholesterol in a subject with low HDL cholesterol levels. Another outcome of treatment is increasing insulin sensivity. Another outcome of treatment may be enhancing glucose tolerance in a subject with glucose intolerance. Yet another outcome of treatment may be decreasing insulin resistance in a subject with increased insulin resistance or elevated levels of insulin. Prevention of diabetes mellitus, in particular diabetes associated with obesity, refers to the administration of a compound or combination of the present invention to prevent the onset of diabetes in a subject in need thereof. A subject in need of preventing diabetes is a prediabetic subject that is overweight or obese.

The term “hypertension” as used herein includes essential, or primary, hypertension wherein the cause is not known or where hypertension is due to greater than one cause, such as changes in both the heart and blood vessels; and secondary hypertension wherein the cause is known. Causes of secondary hypertension include, but are not limited to obesity; kidney disease; hormonal disorders; use of certain drugs, such as oral contraceptives, corticosteroids, cyclosporin, and the like. The term “hypertension” encompasses high blood pressure, in which both the systolic and diastolic pressure levels are elevated, and isolated systolic hypertension, in which only the systolic pressure is elevated to greater than or equal to 140 mm Hg, while the diastolic pressure is less than 90 mm Hg. Normal blood pressure may be defined as below 120 mm Hg (systolic pressure) over 80 mm Hg (diastolic pressure). A hypertensive subject is a subject with hypertension. A pre-hypertensive subject is a subject with a blood pressure that is between 120 mmHg over 80 mmHg and 139 mmHg over 89 mmHg. One outcome of treatment is decreasing blood pressure in a subject with high blood pressure. Treatment of hypertension refers to the administration of the combinations of the present invention to treat hypertension in a hypertensive subject. Prevention of hypertension refers to the administration of the combinations of the present invention to a pre-hypertensive subject to prevent the onset of hypertension or a hypertension related disorder.

The terms “administration of” and or “administering a” compound should be understood to mean providing a compound of the invention or a prodrug of a compound of the invention to a subject in need of treatment. The instant pharmaceutical compositions include administration of a single pharmaceutical dosage formulation which contains an anti-obesity agent and an anti-dyslipidemic agent, as well as administration of each active agent in its own separate pharmaceutical dosage formulation. Where separate dosage formulations are used, the individual components of the composition can be administered at essentially the same time, i.e., concurrently, or at separately staggered times, i.e. sequentially prior to or subsequent to the administration of the other component of the composition. The instant pharmaceutical composition is therefore to be understood to include all such regimes of simultaneous or alternating treatment, and the terms “administration” and “administering” are to be interpreted accordingly. Administration in these various ways are suitable for the present compositions as long as the beneficial pharmaceutical effect of the combination of the anti-obesity agent and the anti-dyslipidemic agent is realized by the patient at substantially the same time. Such beneficial effect is preferably achieved when the target blood level concentrations of each active drug are maintained at substantially the same time. It is preferred that the combination of the anti-obesity agent and the anti-dyslipidemic agent be co-administered concurrently on a once-a-day dosing schedule; however, varying dosing schedules, such as the anti-obesity agent once a day and the anti-dyslipidemic agent once, twice or more times per day, is also encompassed herein. A single oral dosage formulation comprised of both agents in the combination, for example an anti-obesity agent and an anti-dyslipidemic agent, is preferred. A single dosage formulation will provide convenience for the patient, which is an important consideration especially for patients with diabetes, metabolic syndrome, or obese patients who may be in need of multiple medications.

The term “subject”, as used herein refers to an animal, preferably a mammal, most preferably a human, who has been the object of treatment, observation or experiment. In one embodiment the term “mammal” is a “human” said human being either male or female. The instant combinations are also useful for treating or preventing obesity and obesity-related disorders in cats and dogs. As such, the term “mammal” includes companion animals such as cats and dogs.

The term “subject in need thereof” refers to a subject who is in need of treatment or prophylaxis as determined by a researcher, veterinarian, medical doctor or other clinician. In one embodiment, a subject in need thereof is a mammal. In another embodiment, a subject in need thereof is an obese subject. In another embodiment, a subject in need thereof is an obese subject at risk of developing dyslipidemia. In another embodiment, a subject in need thereof is a subject with dyslipidemia. In another embodiment, a subject in need thereof is a subject at risk of developing dyslipidemia. In another embodiment, a subject in need thereof is an obese subject with dyslipidemia. In another embodiment, a subject in need thereof is an obese subject at risk of developing dyslipidemia. In another embodiment, a subject in need thereof is a dyslipidemic subject at risk of developing obesity. In another embodiment, a subject in need thereof is a subject with metabolic syndrome. In another embodiment, a subject in need thereof is an obese subject with metabolic syndrome. In another embodiment, a subject in need thereof is an obese subject at risk of developing metabolic syndrome. In another embodiment, a subject in need thereof is a dyslipidemic subject with metabolic syndrome. In another embodiment, a subject in need thereof is a dyslipidemic subject at risk of developing metabolic syndrome. In another embodiment, a subject in need thereof is an obese dyslipidemic subject with metabolic syndrome. In another embodiment, a subject in need thereof is an obese dyslipidemic subject at risk of developing metabolic syndrome. In another embodiment, a subject in need thereof is a subject with cardiac hypertrophy and/or left ventricular hypertrophy. In another embodiment, a subject in need thereof is an obese subject with cardiac hypertrophy or left ventricular hypertrophy. In another embodiment, a subject in need thereof is a dyslipidemic subject with cardiac hypertrophy or left ventricular hypertrophy. In another embodiment, a subject in need thereof is an obese dyslipidemic subject with cardiac hypertrophy or left ventricular hypertrophy. In another embodiment, a subject in need thereof is an obese dyslipidemic subject at risk of developing cardiac hypertrophy or left ventricular hypertrophy.

The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated. The novel methods of treatment of this invention are for disorders known to those skilled in the art.

The term “prophylactically effective amount” as used herein means the amount of the active compounds in the composition that will elicit the biological or medical response in a tissue, system, subject, or human that is being sought by the researcher, veterinarian, medical doctor or other clinician, to prevent the onset of dyslipidemia, or obesity, or a dyslipidemia-related disorder, or an obesity-related disorder in a subject at risk of developing the disorder.

The magnitude of prophylactic or therapeutic dose of the active ingredients (e.g. the anti-obesity agent, and the anti-dyslipidemic agent) of the composition will, of course, vary with the nature of the severity of the condition to be treated and with the particular compound in the composition and its route of administration. It will also vary according to the age, weight and response of the individual patient. In general, the daily dose range of each compound in the combination lies within the range of from about 0.0001 mg/kg to about 100 mg/kg, preferably from about 0.001 mg/kg to about 50 mg/kg body weight of a subject in single or divided doses. On the other hand, it may be necessary to use dosages outside these limits in some cases.

For use where a composition for intravenous administration is employed, a suitable dosage range is from about 0.0001 mg/kg to about 50 mg/kg, preferably from 0.001 mg/kg to about 20 mg/kg of each compound in the composition per day.

In the case where an oral composition is employed, a suitable dosage range is, e.g. from about 0.001 mg/kg to about 100 mg/kg of each compound in the composition per day, preferably from about 0.01 mg to about 2000 mg per day. For oral administration, the compositions are preferably provided in the form of tablets containing from 0.01 mg to 1,000 mg, e.g. 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850, 1,000 and 2,000 milligrams of each active ingredient for the symptomatic adjustment of the dosage to the subject to be treated. This dosage regimen may be adjusted to provide the optimal therapeutic response.

In general, for treating, controlling, and/or preventing dyslipidemia, dyslipidemia associated with obesity, a dyslipidemia-related disorder, obesity and an obesity-related disorder, the anti-obesity agent in the combination is administered at a daily dosage of from about 0.0001 mg/kg to about 100 mg/kg of body weight, preferably from about 0.001 mg/kg to about 50 mg/kg, given in a single dose or in divided doses two to six times per day, or in sustained release form; and the anti-dyslipidemic agent in the combination is administered at a daily dosage of from about 0.0001 mg/kg to about 100 mg/kg of body weight, preferably from about 0.001 mg/kg to about 50 mg/kg, given in a single dose or in divided doses two to six times per day, or in sustained release form. The dosage regimen may be adjusted to provide the optimal therapeutic response.

In general, for treating, controlling, and/or preventing cardiac hypertrophy, including left ventricular hypertrophy, the anti-obesity agent in the combination is administered at a daily dosage of from about 0.0001 mg/kg to about 100 mg/kg of body weight, preferably from about 0.001 mg/kg to about 50 mg/kg, given in a single dose or in divided doses two to six times per day, or in sustained release form; and the anti-dyslipidemic agent in the combination is administered at a daily dosage of from about 0.0001 mg/kg to about 100 mg/kg of body weight, preferably from about 0.001 mg/kg to about 50 mg/kg, given in a single dose or in divided doses two to six times per day, or in sustained release form. The dosage regimen may be adjusted to provide the optimal therapeutic response.

In general, for treating, controlling, and/or preventing metabolic syndrome, the anti-obesity agent, the anti-dyslipidemic agent, the anti-hypertensive agent, and the anti-diabetic agent in the given combination are administered at a daily dosage of from about 0.0001 mg/kg to about 100 mg/kg of body weight, preferably from about 0.001 mg/kg to about 50 mg/kg, given in a single dose or in divided doses two to six times per day, or in sustained release form. The dosage regimen may be adjusted to provide the optimal therapeutic response.

The compounds of this invention can be administered to humans in the dosage ranges specific for each compound.

Leptin may be administered at a daily dosage of from about 0.01 mg/kg to about 20 mg/kg, preferably, from about 0.01 mg/kg to about 0.3 mg/kg, preferably injected in a single dose or in divided doses.

Nalmefene may be administered at a daily dosage of from about 0.0001 mg/kg to about 10 mg/kg, preferably from about 0.001 to about 0.05 mg/kg.

Orlistat may be administered at a daily dosage of from about 20 mg to about 1200 mg, preferably from about 120 mg to 400 mg in a single dose or divided doses 2 to 3 times per day or in sustained release form; more preferably a 120 mg single dose 3 times per day, or in sustained release form.

Sibutramine may be administered at a daily dosage of from about 0.01 mg/kg to about 10 mg/kg, preferably from about 0.01 mg/kg to about 1 mg/kg in a single dose or in divided doses 2 to 3 times per day, or in sustained release form; more preferably the single daily dose of sibutramine is 5 mg, 10 mg, 15 mg, 20 mg or 30 mg orally.

Rimonabant may be administered at a daily dosage of from about 0.01 mg/kg to about 8 mg/kg, more preferably from about 0.3 mg/kg to about 3 mg/kg of body weight in a single dose or in divided doses 2 to 3 times per day, or in sustained release form.

Topiramate (Topamax®) may be administered at a daily dosage of from about 10 mg to about 1,600 mg per day, preferably from about 50 mg to about 400 mg per day in a single dose or in divided doses, or in sustained release form.

Specific dosages for commercially available anti-dyslipidemic agents which are useful in the present invention may be found in the Physician's Desk Reference, Edition 56 (2002).

The effective dosage of each of the active ingredients employed in the composition may vary depending on the particular compound employed, the mode of administration, the condition being treated and the severity of the condition being treated. Thus, the dosage regimen utilizing the compositions of the present invention is selected in accordance with a variety of factors including type, species, age, general health, body weight, diet, sex and medical condition of the subject; the severity of the condition to be treated; the renal and hepatic function of the patient; the drug combination; and the particular compounds employed and their routes of administration. A physician, clinician or veterinarian of ordinary skill can readily determine and prescribe the effective amount of the drug required to prevent, counter or arrest the progress of the condition.

The weight ratio of the agents in the combinations of the present invention (e.g. the anti-obesity agent; the anti-dyslipidemic agent) may be varied and will depend upon the effective dose of each ingredient. Generally, an effective dose of each will be used. Thus, for example, when an anti-obesity agent, such as an NPY5 antagonist, is combined with an anti-dyslipidemic agent, such as a HMG-CoA reductase inhibitor like simvastatin the weight ratio of the NPY5 antagonist to the HMG-CoA reductase inhibitor will generally range from about 1000:1 to about 1:1000, preferably about 200:1 to about 1:200. Compositions of the agents in the combinations of the present invention (e.g. the anti-obesity agent; the anti-dyslipidemic agent) will generally also be within the aforementioned range, but in each case, an effective dose of each active ingredient should be used.

Another aspect of the present invention provides pharmaceutical compositions comprising a pharmaceutical carrier and a therapeutically effective amount of each compound in the composition of the present invention. The term “composition”, as in pharmaceutical composition, is intended to encompass a product comprising the active ingredient(s), and the inert ingredient(s), such as pharmaceutically acceptable excipients, that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing an anti-obesity agent and an anti-dyslipidemic agent, and pharmaceutically acceptable excipients.

Any suitable route of administration may be employed for providing a subject, especially a human, with an effective dosage of a composition of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a combination of one or more anti-obesity agents, and one or more anti-dyslipidemic agents, as active ingredients or a pharmaceutically acceptable salt or ester thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compounds suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. These compositions may be conveniently presented in unit dosage form and prepared by any of the methods well-known in the art of pharmacy.

For administration by inhalation, the compositions of the present invention are conveniently delivered in the form of an aerosol spray presentation from pressurized packs or nebulizers. The compositions may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of the instant composition in suitable propellants, such as fluorocarbons or hydrocarbons and dry powder inhalation (DPI) aerosol, which may be formulated as a dry powder of the composition with or without additional excipients.

Suitable topical formulations of the compositions of the present invention include transdermal devices, aerosols, creams, solutions, ointments, gels, lotions, dusting powders, and the like. The topical pharmaceutical compositions containing the compositions of the present invention ordinarily include about 0.005% to 5% by weight of the active compounds in admixture with a pharmaceutically acceptable vehicle. Transdermal skin patches useful for administering the compositions of the present invention include those well known to those of ordinary skill in that art. To be administered in the form of a transdermal delivery system, the dosage administration will, of course be continuous rather than intermittent throughout the dosage regimen.

The compositions of the present invention can also be administered in the form of liposome delivery systems, such as small unilamellar vesicles, large unilamellar vesicles and multilamellar vesicles. Liposomes can be formed from a variety of phospholipids, such as cholesterol, sterylamine or phosphatidylcholines.

Compositions of the present invention may also be delivered by the use of monoclonal antibodies as individual carriers to which the compound molecules are coupled. The compounds in these compositions may also be coupled with soluble polymers as targetable drug carriers. Such polymers can include polyvinylpyrrolidone, pyran copolymer, polyhydroxypropyl-methacrylamide phenol, polyhydroxyethylasparamidepheon, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxybutyric acid, polyorthoesters, polyacetals, polydihydropyrans, polycyanoacrylates and cross-linked or amphipathic block copolymers of hydrogels.

Compositions of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

In practical use, each compound in the compositions of the present invention (e.g. each anti-obesity agent; each anti-dyslipidemic agent) can be combined as the active ingredients in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, pellet, powder and tablets, with the solid oral preparations being preferred over the liquid preparations. Because of their ease of administration, tablets and capsules represent the typical oral dosage unit form, in which case solid pharmaceutical carriers are obviously employed. If desired, tablets may be coated by standard aqueous or nonaqueous techniques.

In addition to the common dosage forms set out above, the composition may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules (including timed release and sustained release formulations), pills, cachets, powders, granules or tablets each containing a predetermined amount of the active ingredients, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water, oils and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. When a dosage unit form is a capsule, it may contain, in addition to materials of the above type, a liquid carrier such as a fatty oil.

Desirably, each tablet contains from 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient in the composition of the present invention (e.g. each anti-obesity agent, each anti-dyslipidemic agent, each anti-hypertensive agent, each anti-diabetic agent) for the symptomatic adjustment of the dosage to the subject to be treated; and each cachet or capsule contains from about 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient in the composition of the present invention (e.g. each anti-obesity agent, each anti-dyslipidemic agent, each anti-hypertensive agent, each anti-diabetic agent) for the symptomatic adjustment of the dosage to the subject to be treated.

Exemplifying the invention is a pharmaceutical composition comprising an anti-obesity agent and an anti-dyslipidemic agent described above and a pharmaceutically acceptable carrier.

Also exemplifying the invention is a pharmaceutical composition made by combining any of the anti-obesity agents and anti-dyslipidemic agents described above and a pharmaceutically acceptable carrier. An illustration of the invention is a process for making a pharmaceutical composition comprising combining any of the anti-obesity agents and anti-dyslipidemic agents described above and a pharmaceutically acceptable carrier.

The dose may be administered in a single daily dose or the total daily dosage may be administered in divided doses of two to six times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compositions of the present invention:

| Injectable Suspension (I.M.) | mg/mL |
|---|---|
| NPY5 antagonist of Formula I or II | 0.70 |
| Simvastatin | 1.0 |
| cyclodextrin | Q.S. ed to |
| (35% weight/volume) | 1 ml volume |
| glycerol | 63.05 |

Water for injection to a total volume of 1 mL

| Tablet | mg/tablet |
|---|---|
| NPY5 antagonist of Formula I or II | 25 |
| Simvastatin | 20 |
| Microcrystalline Cellulose | 40.5 |
| Lactose | 101.5 |
| Croscarmellose Sodium | 5.0 |
| Hydroxypropylcellulose | 6.0 |
| Sodium Dodecyl Sulfate | 1.0 |
| Magnesium Stearate | 1.0 |
| | 200 |

| Capsule | mg/capsule |
|---|---|
| NPY5 antagonist of Formula I or II | 100 |
| Simvastatin | 20 |
| Lactose | 65 |
| Sodium Dodecyl Sulfate | 15 |
| | 200 |

| Aerosol | Per canister | |
|---|---|---|
| NPY5 antagonist of Formula I or II | 4 | mg |
| Simvastatin | 9 | mg |
| Lecithin, NF Liq. Conc. | 1.2 | mg |
| Trichlorofluoromethane, NF | 4.025 | g |
| Dichlorodifluoromethane, NF | 12.15 | g |

It will be understood that the scope of compositions of the compounds of this invention with other agents useful for treating or preventing obesity and obesity-related conditions includes in principle any combination with any pharmaceutical composition useful for treating obesity and obesity-related disorders.

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

### EXAMPLE 1

In vivo study for combination therapy with a NPY5 antagonist and an anti-dyslipidemic agent (effect on obesity/food intake and lipids)

DIO mice are treated simultaneously with an effective dose of a NPY5 antagonist and an effective dose of an anti-dyslipidemic agent.

Materials and Methods

Male C57BL/6J mice (CLEA Japan Inc., 12-16 months old at the beginning of the drug administration) are used. Mice are given water and regular pellet chow (CE-2, CLEA Japan Inc.) ad libitum. They are kept in an animal room which is maintained at 23±2° C. temperature, 55±15% relative humidity and on a 12-hr light-dark cycle (7:00-19:00) during a quarantine and acclimatization period of 1 week. Before the start of drug administration, mice are fed a MHF diet (Oriental BioService Co., Tokyo, Japan) for about 9 to 10 months until the body weight gain reaches a plateau. After the body weight gain reaches a plateau, the diet is changed to a powder MHF diet. The powder MHF diet is given by powder feeder (small dishes). Diet and dishes are changed everyday, and daily food intake is measured. During this period, animals are orally administered vehicle (0.5% methylcellulose in distilled water) by gavage once-daily. After the stable feeding is observed, the amount of new food is adjusted to daily food intake +0.3 g, to minimize the amount of spilled food. After the acclimation period, the MHF diet-fed mice are divided into two groups to match average values of body weight and food intake (n=8-12). One of the groups is orally administered vehicle while the second group is administered a combination of compound A, a NPY5 antagonist anti-obesity agent, and simvastatin, an anti-dyslipidemic agent, both at a dose of 100 mg/kg once-daily for 1.5 months by gavage, respectively. The administration is done one and half hours before the beginning of the dark period following the measurement of body weight. The amount of food is divided into two meals and given at about 8:00 and 18:00 to avoid a long duration of fasting. Food and body weight are measured. At the end of the treatment period, serum cholesterol and lipds are determined.

Effective combinations result in body weight loss of at least 5% and a statistically significant reduction in total cholesterol, LDL, or triglycerides in the treated group compared to the vehicle treated group.

### EXAMPLE 2

Human study for combination therapy with a NPY5 antagonist and an anti-dyslipidemic agent (effect on obesity/food intake and lipids)

Materials and Methods

800 people with a BMI≧30 are advised to diet and increase their physical activity. After a two-week placebo run-in period, which includes a standardized program of diet, physical activity, and lifestyle changes, the patients are randomized into 2 treatment groups: placebo and a second group is administered a combination of a NPY5 antagonist anti-obesity agent, such as for instance 100 mg/kg of compound A, and an anti-dyslipidemic agent, such as 20 mg of simvastatin. The NPY5 antagonist is given once per day, as previously determined to be effective. The anti-dyslipidemic agent is given once or more per day, as previously determined to be effective. Patients are treated for 6 months, body weights are measured biweekly, and appetite, hunger, satiety are measured weekly using standard questionnaires. At the end of the treatment period, serum cholesterol and lipids are determined.

Effective combinations result in body weight loss of ≧5% or a decrease in food intake) and a statistically significant reduction in total cholesterol, LDL, or triglycerides in the treated group compared to the vehicle treated group.

### EXAMPLE 3

Human study for combination therapy with a NPY5 antagonist and an anti-dyslipidemic agent (effect on Cardiac Hypertrophy and Left Ventricular Hypertrophy

Materials and Methods

800 people with a BMI≧30 are advised to diet and increase their physical activity. After a two-week placebo run-in period, which includes a standardized program of diet, physical activity, and lifestyle changes, the patients are randomized into 4 treatment groups: placebo; an effective dose of a NPY5 antagonist, such as 1000 mg of Compound A; an effective dose of an anti-dyslipidemic agent such as simvastatin; and an effective dose of the NPY5 antagonist plus an effective dose of the anti-dyslipidemic agent. The NPY5 antagonist is given once or more per day, as previously determined to be effective. The anti-dyslipidemic agent is given once or more per day, as previously determined to be effective. Patients are treated for 6 months, body weights are measured biweekly, and appetite, hunger, satiety are measured weekly using standard questionnaires. Echocardiographic evaluation of the heart including left ventricular mass is performed at randomization and at the end of the study.

Effective combinations result in body weight loss of ≧5% and a statistically significant decrease in left ventricular mass.

### EXAMPLE 4

Non Diabetic Rodent Model of Syndrome X: study for combination therapy with a NPY5 antagonist and an anti-dyslipidemic agent and/or an anti-diabetic agent and/or an anti-hypertensive agent. (Effect blood pressure, serum insulin levels, triglyceride levels, and fatty acid levels)

The following experiment demonstrates the ability of the composition to lower blood pressure in an animal model of Syndrome X. This experiment uses a non-diabetic rodent model where blood insulin levels, blood pressure and serum triglycerides are elevated but serum glucose levels are within normal limits.

Materials and Methods

Male, Sprague-Dawley rats (Harlan Sprague Dawley, Indianapolis, Ind.), initially weighing 175-199 g are used for all experiments. Prior to dietary manipulation, all rats are fed Purina Rat Chow (no. 5012; St. Louis, Mo.) and water ad libitum and maintained on a 12-h (0600-1800 h) light-dark cycle. The rats are then placed on a diet (TD 78463; Harlan Teklad, Madison, Wis.) which provides 60% of total calories as fructose. The fructose-enriched diet is given for 11 days, during which time the rats are acclimated to the procedure of blood pressure measurement. Ambient temperature is kept at 30 C. The equipment used includes magnetic animal holders connected with manual scanner (model 65-12, IITC, Inc., Woodland Hills, Calif.), pulse amplifier (model 59, IITC, Inc.), and dual-channel recorder (model 1202, Linear Intrs. Corp., Reno, Nev.).

At the end of the initial dietary period, blood pressure is determined, and rats randomly divided into two groups. Both groups are maintained on the fructose-enriched diet, but one group is gavaged with a combination of anti-obesity such as compound A (such as 100 mpk PO) and an antidyslipidemic agent such as simvastatin, and/or an anti-diabetic agent such as pioglitazone, and/or an anti-hypertensive agent such as enalapril, whereas the other group is treated in the same manner with vehicle alone. Blood pressure is measured once per week, before and after doses of either the combination or vehicle (8 weeks of treatment). In both instances, the general procedure is similar. Rats are removed from the animal room and taken to the laboratory at 0900 h. They are allowed free access to water and are kept in a quiet area before the blood pressure is measured at 1300 h. The tail-cuff method, without external preheating, is used to measure the systolic blood pressure. The systolic blood pressure is measured in the conscious state and has been shown with this technique to be similar to that obtained by direct arterial cannulation. The final blood pressure determinations were performed on the afternoon following the last morning dose of the combination or vehicle. In approximately half of the rats studied, tail vein blood is removed at 1300 h (four hours after removal of food), centrifuged, frozen, and later assayed for plasma glucose, insulin, and triglyceride concentrations. Plasma free fatty acid concentration is assayed enzymatically by the ACS-ACOD method using a commercial kit (Waro Chemicals Inc., Richmond, Va.).

The animal model used in this example has many of the features of Syndrome X. Fructose fed rats do not have increased blood glucose and therefore this is not a diabetic model. However, these rats do show increased serum insulin, increased triglycerides and free fatty acid concentration and increased blood pressure. Thus, this animal model is the animal model for Syndrome X.

Effective compositions improve the characteristic cluster of symptoms associated with Syndrome X. Effective compositions lower at least two of the symptonms of Syndrome X: blood pressure, blood insulin, free fatty acid, bodyweight and triglyceride levels in a non-diabetic rat model where blood glucose levels remain normal.

Additional animal models can be used, including BRS3 KO mice (Ohki-Hamazki et al, Nature 390: 165, 1997) and diet-induced obese and hypertensive dogs (Hall et al, Am. J. Hypertension, 2001, 14: 103S-115S).

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A composition comprising an anti-obesity agent, and pharmaceutically acceptable salts and esters thereof, and an anti-dyslipidemic agent, and pharmaceutically acceptable salts and esters thereof.

2. The composition of claim 1 comprising (a) an anti-obesity agent selected from the group consisting of
(1) a 5HT transporter inhibitor,
(2) a NE transporter inhibitor,
(3) a CB-1 antagonist/inverse agonist,
(4) a ghrelin antibody,
(5) a ghrelin antagonist,
(6) a H3 antagonist/inverse agonist,
(7) a MCH1R antagonist,
(8) a MCH2R agonist/antagonist,
(9) a NPY1 antagonist,
(10) a NPY2 agonist,
(11) a NPY5 antagonist,
(12) leptin,
(13) a leptin derivative,
(14) an opioid antagonist,
(15) an orexin antagonist,
(16) a BRS3 agonist,
(17) a CCK-A agonist,
(18) a CNTF,
(19) a CNTF derivative,
(20) a GHS agonist,
(21) 5HT2c agonist,
(22) a GLP-1 agonist,
(23) a DP-IV inhibitor,
(24) a Mc3r agonist,
(25) a Mc4r agonist,
(26) a monoamine reuptake inhibitor,
(27) a serotonin reuptake inhibitor,
(28) topiramate,
(29) phytopharm compound 57,
(30) an ACC2 inhibitor,
(31) a β3 agonist,
(32) a DGAT1 inhibitor,
(33) a DGAT2 inhibitor,
(34) a FAS inhibitor,
(35) a PDE inhibitor,
(36) a thyroid hormone β agonist,
(37) an UCP-1, 2, or 3 activator,
(38) an acyl-estrogen,
(39) a glucocorticoid antagonist,
(40) an 11β HSD-1 inhibitor,
(41) a SCD-1 inhibitor,
(42) a lipase inhibitor,
(43) a fatty acid transporter inhibitor,
(44) a dicarboxylate transporter inhibitor,
(45) a glucose transporter inhibitor, and
(46) a phosphate transporter inhibitor,
and pharmaceutically acceptable salts and esters thereof; and
(b) an anti-dyslipidemic agent selected from the group consisting of
(1) a bile acid sequesterant,
(2) a HMG-CoA reductase inhibitor,
(3) a HMG-CoA synthase inhibitor,
(4) a cholesterol absorption inhibitor,
(5) an acyl coenzyme A-cholesterol acyl transferase inhibitor,
(6) a cholesteryl ester transfer protein inhibitor,
(7) a squalene synthetase inhibitor,
(8) an anti-oxidant,
(9) a PPAR α agonist,
(10) a FXR receptor modulator,
(11) a LXR receptor agonist,
(12) a lipoprotein synthesis inhibitor,
(13) a renin angiotensin system inhibitor,
(14) a microsomal triglyceride transport inhibitor,
(15) a bile acid reabsorption inhibitor,
(16) a PPARδ agonist,
(17) a triglyceride synthesis inhibitor,
(18) a transcription modulator,
(19) a squalene epoxidase inhibitor,
(20) a low density lipoprotein receptor inducer,
(21) a platelet aggregation inhibitor,
(22) a 5-LO or FLAP inhibitor,
(23) a PPAR δ partial agonist, and
(24) niacin or a niacin receptor agonist,
and pharmaceutically acceptable salts and esters thereof.

3. The composition of claim 2 wherein the anti-obesity agent is a NPY5 antagonist, or a pharmaceutically acceptable salt or ester thereof.

4. The composition of claim 3 wherein the NPY5 antagonist is selected from the group consisting of a compound of formula I 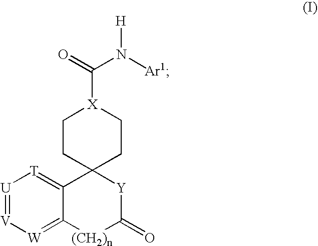
and pharmaceutically acceptable salts and esters thereof, wherein
Ar¹ is selected from the group consisting of:
(1) aryl, and
(2) heteroaryl,
wherein the aryl and heteroaryl groups are unsubstituted or optionally substituted with a substituent selected from the group consisting of:
(a) halogen,
(b) nitro,
(c) lower alkyl,
(d) halo(lower)alkyl,
(e) hydroxy(lower)alkyl,
(f) cyclo(lower)alkyl,
(g) lower alkenyl,
(h) lower alkoxy,
(i) halo(lower)alkoxy,
(j) lower alkylthio,
(k) carboxyl,
(l) lower alkanoyl,
(m) lower alkoxycarbonyl,
(n) lower alkylene optionally substituted with oxo, and
(o) -Q-Ar²;
Ar² is selected from the group consisting of
(1) aryl, and
(2) heteroaryl,
wherein aryl and heteroaryl are unsubstituted or optionally substituted with a substituent selected from the group consisting of:
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl;
n is 0 or 1;
Q is selected from the group consisting of a single bond or carbonyl;
T, U, V and W are each independently selected from the group consisting of
(1) nitrogen, and
(2) methine,
wherein the methine group is unsubstituted or optionally substituted with a substituent selected from the group consisting of
(a) halogen,
(b) lower alkyl,
(c) hydroxy, and
(d) lower alkoxy; and
wherein at least two of T, U, V, and W are methine;
X is selected from the group consisting of
(1) nitrogen, and
(2) methine; and
Y is selected from the group consisting of
(1) imino, unsubstituted or optionally substituted with lower alkyl, and
(2) oxygen.

5. The composition of claim 4 wherein the NPY5 antagonist is selected from the group consisting of:
(1) N-(4-benzoylphenyl)-3-oxospiro[isoindoline-1,4′-piperidine]-1′-carboxamide;
(2) 3-oxo-N-(5-phenyl-2-pyrazinyl)spiro[isoindoline-1,4′-piperidine]-1′-carboxamide;
(3) N-(7-methyl-2-quinolyl)-3-oxospiro[isoindoline-1,4′-piperidine]-1′-carboxamide;
(4) N-(4-benzoylphenyl)-2-methyl-3-oxospiro[isoindoline-1,4′-piperidine]-1′-carboxamide;
(5) N-(4-benzoylphenyl)-3,4-dihydro-3-oxospiro[isoquinoline-1(2H),4′-piperidine]-1′-carboxamide;
(6) 3,4-dihydro-3-oxo-N-(5-phenyl-2pyrazinyl)spiro-[isoquinoline-1(2H),4′-piperidine]-1′-carboxamide;
(7) 3,4-dihydro-N-(7-methyl-2-quinolyl)-3-oxospiro-[isoquinoline-1(2H),4′-piperidine]-1′-carboxamide;
(8) N-(4-acetylphenyl)-3,4-dihydro-3-oxospiro-[isoquinoline-1(2H),4′-piperidine]-1′-carboxamide;
(9) 3,4-dihydro-3-oxo-N-[1-(2-quinolyl)-4-imidazolyl]-spiro[isoquinoline-1(2H),4′-piperidine]-1′-carboxamide;
(10) 3,4-dihydro-3-oxo-N-(5-oxo-5,6,7,8-tetrahydro-2-naphthyl)spiro[isoquinoline-1(2H),4′-piperidine]-1′-carboxamide;
( 11) 3,4-dihydro-N-[5-(2-methyl-1-propenyl)-2-pyrazinyl]-3-oxospiro[isoquinoline-1(2H),4′-piperidine]-1′-carboxamide;
(12) 3,4-dihydro-3-oxo-N-(3-phenyl-5-isoxazolyl)spiro-[isoquinoline-1(2H),4′-piperidine]-1′-carboxamide;
(13) N-[1-(7-benzo[b]furanyl)4-imidazolyl]-3,4-dihydro-3-oxospiro[isoquinoline-1(2H),4′-piperidine]-1′-carboxamide;
(14) N-[1-(3-difluoromethoxyphenyl)4-imidazolyl]-3,4-dihydro-3-oxospiro[isoquinoline-1(2H),4′-piperidine]-1′-carboxamide;
(15) 3,4-dihydro-3-oxo-N-[4-(2-pyridylcarbonyl)phenyl]-spiro[isoquinoline-1(2H),4′-piperidine]-1′-carboxamide;
(16) N-(3,4-dichlorophenyl)-3,4-dihydro-3-oxospiro-[isoquinoline-1(2H),4′-piperidine]-1′-carboxamide;
(17) N-[1-(3-chlorophenyl)-4-imidazolyl]-3,4-dihydro-3-oxospiro[isoquinoline-1(2H),4′-piperidine]-1′-carboxamide;
(18) 3,4-dihydro-3-oxo-N-(5-phenyl-2-thiazolyl)spiro-[isoquinoline-1(2H),4′-piperidine]-1′-carboxamide;
(19) 3,4-dihydro-3-oxo-N-[5-(2-pyridyl)-2-pyrazinyl]spiro-[isoquinoline-1(2H),4′-piperidine]-1′-carboxamide;
(20) 3,4-dihydro-N-(4-methyl-2-benzothiazolyl)-3-oxospiro-[isoquinoline-1(2H),4′-piperidine]-1′-carboxamide;
(21) N-(5-chloro-2-benzoxazolyl)-3,4-dihydro-3-oxospiro-[isoquinoline-1(2H),4′-piperidine]-1′-carboxamide;
(22) N-(4-benzoylphenyl)-3-oxospiro[isobenzofuran-1(3H),4′-piperidine]-1′-carboxamide;
(23) 3-oxo-N-(5-phenyl-2-pyrazinyl)-spiro[isobenzofuran-1(3H),4′-piperidine]-1′-carboxamide;
(24) N-(7-methyl-2-quinolyl)-3-oxospiro[isobenzofuran-1(3H),4′-piperidine]-1′-carboxamide;
(25) 3-oxo-N-(3-phenyl-5-isoxazolyl)spiro[isobenzofuran-1(3H),4′-piperidine]-1′-carboxamide;
(26) 3-oxo-N-(7-trifluoromethylpyrido[3,2-b]pyridin-2-yl)spiro[isobenzofuran-1(3H),4′-piperidine]-1′-carboxamide;
(27) 3-oxo-N-(5-phenyl-2-pyrimidinyl)spiro[isobenzofuran-1(3H),4′-piperidine]-1′-carboxamide;
(28) 3-oxo-N-[1-(3-quinolyl)-4-imidazolyl]spiro[isobenzofuran-1(3H),4′-piperidine]-1′-carboxamide;
(29) 3-oxo-N-(5-phenyl-3-pyrazolyl)spiro[isobenzofuran-1(3H),4′-piperidine]-1′-carboxamide;
(30) N-[5-(4-chlorophenyl)-3-pyrazolyl]-3-oxospiro-[isobenzofuran-1(3H),4′-piperidine]-1′-carboxamide;
(31) 3-oxo-N-[5-(3-quinolyl)-3-pyrazolyl]spiro[isobenzofuran-1(3H),4′-piperidine]-1′-carboxamide;
(32) N-[5-(3-fluorophenyl)-2-pyrimidinyl]-3-oxospiro-[isobenzofuran-1(3H),4′-piperidine]-1′-carboxamide;
(33) 3-oxo-N-[5-(3-trifluoromethylphenyl)-2-pyrimidinyl]-spiro[isobenzofuran-1(3H),4′-piperidine]-1′-carboxamide;
(34) N-[5-(3-chlorophenyl)-2-pyrimidinyl]-3-oxospiro-[isobenzofuran-1(3H),4′-piperidine]-1′-carboxamide;
(35) N-(7-difluoromethoxypyrido[3,2-b]pyridin-2-yl)-3-oxospiro[isobenzofuran-1(3H),4′-piperidine]-1′-carboxamide;
(36) 3-oxo-N-(5-phenyl-1,2,4-thiadiazol-3-yl)spiro-[isobenzofuran-1(3H),4′-piperidine]-1′-carboxamide;
(37) N-{1-[3-(2-hydroxyethyl)phenyl]-4-imidazolyl}-3-oxospiro-[isobenzofuran-1(3H),4′-piperidine]-1′-carboxamide;
(38) N-[4-(1-ethyl-2-imidazolyl)phenyl]-3-oxospiro-[isobenzofuran-1(3H),4′-piperidine]-1′-carboxamide;
(39) N-[1-(3-methoxyphenyl)-4-imidazolyl]-3-oxospiro-[isobenzofuran-1(3H),4′-piperidine]-1′-carboxamide;
(40) 6-fluoro-3-oxo-N-(5-phenyl-2-pyrazinyl)spiro-[isobenzofuran-1(3H),4′-piperidine]-1′-carboxamide;
(41) 6-fluoro-3-oxo-N-(5-phenyl-2-pyrimidinyl)spiro-[isobenzofuran-1(3H),4′-piperidine]-1′-carboxamide;
(42) 5-fluoro-3-oxo-N-(5-phenyl-2-pyrazinyl)spiro[isobenzofuran-1(3H),4′-piperidine]-1′-carboxamide;
(43) 5-fluoro-3-oxo-N-(5-phenyl-2-pyrimidinyl)spiro-[isobenzofuran-1(3H),4′-piperidine]-1′-carboxamide;
(44) N-(4-benzoylphenyl)-3,4-dihydro-3-oxospiro[1H-2-benzopyran-1,4′-piperidine]-1′-carboxamide;
(45) 3,4-dihydro-3-oxo-N-(5-phenyl-2-pyrazinyl)spiro[1H-2-benzopyran-1,4′-piperidine]-1′-carboxamide;
(46) N-(5-benzoyl-2-pyrazinyl)-3,4-dihydro-3-oxospiro[1H-2-benzopyran-1,4′-piperidine]-1′-carboxamide;
(47) trans-N-(4-benzoylphenyl)-3′-oxospiro[cyclohexane-1,1′(3′H)-isobenzofuran]-4-carboxamide;
(48) trans-3′-oxo-N-(5-phenyl-2-pyrazinyl)spiro[cyclohexane-1,1′(3′H)-isobenzofuran]4-carboxamide;
(49) trans-3′-oxo-N-(1-phenyl-4-imidazolyl)spiro[cyclohexane-1,1′(3′H)-isobenzofuran]-4-carboxamide;
(50) trans-3′-oxo-N-(5-phenyl-2-pyrimidinyl)spiro[cyclohexane-1,1′(3′H)-isobenzofuran]-4-carboxamide;
(51) trans-N-[1-(3,5-difluorophenyl)-4-imidazolyl]-3′-oxospiro-[cyclohexane-1,1′(3′H)-isobenzofuran]-4-carboxamide;
(52) trans-3′-oxo-N-(5-phenyl-3-pyrazolyl)spiro[cyclohexane-1,1′(3′H)-isobenzofuran]-4-carboxamide;
(53) trans-N-[1-(2-fluorophenyl)-4-imidazolyl]-3′-oxospiro-[cyclohexane-1,1′(3′H)-isobenzofuran]-4-carboxamide;
(54) trans-N-(4-acetyl-3-trifluoromethylphenyl)-3′-oxospiro-[cyclohexane-1,1′(3′H)-isobenzofuran]-4-carboxamide;
(55) trans-3′-oxo-N-[1-(3-quinolyl)-4-imidazolyl]-spiro[cyclohexane-1,1′(3′H)-isobenzofuran]-4-carboxamide;
(56) trans-N-[1-(3-cyanophenyl)-4-imidazolyl]-3′-oxospiro-[cyclohexane-1,1′(3′H)-isobenzofuran]-4-carboxamide;
(57) trans-N-(4-benzoylphenyl)-3-oxospiro[4-azaisobenzofuran-1(3H),1′-cyclohexane]-4′-carboxamide;
(58) trans-3-oxo-N-(5-phenyl-2-pyrazinyl)spiro[4-azaisobenzofuran-1(3H),1′-cyclohexane]-4′-carboxamide;
(59) trans-3-oxo-N-(3-phenyl-5-isoxazolyl)spiro[4-azaisobenzofuran-1(3H),1′-cyclohexane]-4′-carboxamide;
(60) trans-3-oxo-N-(5-phenyl-2-pyrimidinyl)spiro[4-azaisobenzofuran-1(3H),1′-cyclohexane]-4′-carboxamide;
(61) trans-N-(4-benzoylphenyl)-3-oxospiro[5-azaisobenzofuran-1(3H),1′-cyclohexane]-4′-carboxamide;
(62) trans-N-(4-benzoylphenyl)-3-oxospiro[6-azaisobenzofuran-1(3H),1′-cyclohexane]-4′-carboxamide;
(63) N-[5-(4-hydroxyphenyl)-2-pyrazinyl]-3-oxospiro[isobenzofuran-1(3H),4′-piperidine]-1′-carboxamide;
(64) N-[5-(3-hydroxyphenyl)-2-pyrazinyl]-3-oxospiro[isobenzofuran-1(3H),4′-piperidine]-1′-carboxamide;
(65) 4-fluoro-3-oxo-N-(5-phenyl-2-pyrimidinyl)spiro[isobenzofuran-1(3H),4′-piperidine]-1′-carboxamide;
(66) 7-fluoro-3-oxo-N-(5-phenyl-2-pyrimidinyl)spiro[isobenzofuran-1(3H),4′-piperidine]-1′-carboxamide;
(67) 6-ethyl-3-oxo-N-(5-phenyl-2-pyrazinyl)spiro[isobenzofuran-1(3H),4′-piperidine]-carboxamide;
(68) 6-hydroxy-3-oxo-N-(5-phenyl-2-pyrazinyl)spiro[isobenzofuran-1(3H),4′-piperidine]-1′-carboxamide;
(69) trans-3-oxo-N-(5-phenyl-2-pyrimidinyl)spiro[5-azaisobenzofuran-1(3H),1′-cyclohexane]-4′-carboxamide;
(70) trans-N-[5-(3-fluorophenyl)-2-pyrimidinyl-3-oxospiro[5-azaisobenzofuran-1(3H),1′-cyclohexane]-4′-carboxamide;
(71) trans-N-[5-(2-fluorophenyl)-2-pyrimidinyl]-3-oxospiro[5-azaisobenzofuran-1(3H),1′-cyclohexane]-4′-carboxamide;
(72) trans-3-oxo-N-(4-phenyl-2-oxazolyl)spiro[5-azaisobenzofuran-1(3H),1′-cyclohexane]-4′-carboxamide;
(73) trans-N-[5-(2-methylphenyl)-2-pyrimidinyl]-3-oxospiro[5-azaisobenzofuran-1(3H),1′-cyclohexane]-4′-carboxamide;
(74) trans-N-[5-(3-methylphenyl)-2-pyrimidinyl]-3-oxospiro[5-azaisobenzofuran-1(3H),1′-cyclohexane]-4′-carboxamide;
(75) trans-N-[5-(3-fluoromethoxyphenyl)-2-pyrimidinyl]-3-oxospiro[5-azaisobenzofuran-1(3H),1′-cyclohexane]-4′-carboxamide;
(76) trans-N-[5-(3-fluoromethylphenyl)-2-pyrimidinyl]-3-oxospiro[5-azaisobenzofuran-1(3H),1′-cyclohexane]-4′-carboxamide;
(77) trans-N-[5-(3-fluoro-5-methoxyphenyl)-2-pyrimidinyl]-3-oxospiro[5-azaisobenzofuran-1(3H),1′-cyclohexane]-4′-carboxamide;
(78) trans-N-[5-(2-fluoro-5-methylphenyl)-2-pyrimidinyl]-3-oxospiro[5-azaisobenzofuran-1(3H),1′-cyclohexane]-4′-carboxamide;
(79) trans-N-[4-(3-fluoromethoxyphenyl)-2-oxazolyl]-3-oxospiro[5-azaisobenzofuran-1(3H),1′-cyclohexane]-4′-carboxamide;
(80) trans-N-[5-(3-hydroxymethylphenyl)-2-pyrimidinyl]-3-oxospiro[5-azaisobenzofuran-1(3H),1′-cyclohexane]-4′-carboxamide;
(81) trans-N-[5-(3-hydroxyphenyl)-2-pyrimidinyl]-3-oxospiro[5-azaisobenzofuran-1(3H),1′-cyclohexane]-4′-carboxamide;
(82) trans-3-oxo-N-(5-phenyl-2-pyrimidinyl)spiro[6-azaisobenzofuran-1(3H), 1′-cyclohexane]-4′-carboxamide;
(83) trans-N-[5-(3-fluoromethylphenyl)-2-pyrimidinyl]-3-oxospiro[6-azaisobenzofuran-1(3H),1′-cyclohexane]-4′-carboxamide;
(84) trans-N-[5-(3-fluoromethoxyphenyl)-2-prymidinyl]-3-oxospiro[6-azaisobenzofuran-1(3H),1′-cyclohexane]-4′-carboxamide;
(85) trans-3-oxo-N-(6-phenyl-1,2,4-triazin-3-yl)spiro[6-azaisobenzofuran-1(3H), 1′-cyclohexane]-4′-carboxamide;
(86) trans-N-[5-(2-difluoromethoxyphenyl)-3-pyrazolyl]-3-oxospiro[6-azaisobenzofuran-1(3H), 1′-cyclohexane]-4′-carboxamide;
(87) trans-N-[5-(3-difluoromethoxyphenyl)-3-pyrazolyl]-3-oxospiro[6-azaisobenzofuran-1(3H),1′-cyclohexane]-4′-carboxamide;
(88) trans-N-[5-(3-fluorophenyl)-3-pyrazolyl]-3-oxospiro[6-azaisobenzofuran-1(3H),1′-cyclohexane]-4′-carboxamide;
(89) trans-N-[5-(4-fluorophenyl)-3-pyrazolyl]-3-oxospiro[6-azaisobenzofuran-1(3H),1′-cyclohexane]-4′-carboxamide;
(90) trans-N-(4-benzoylphenyl)-3-oxospiro[7-azaisobenzofuran-1(3H),1′-cyclohexane]-4′-carboxamide;
(91) trans-N-[1-(3,5-difluorophenyl)4-imidazolyl]-3-oxospiro[7-azaisobenzofuran-1(3H),1′-cyclohexane]-4′-carboxamide;
(92) trans-3-oxo-N-[2-phenyl4-pyridyl]spiro[7-azaisobenzofuran-1(3H),1′-cyclohexane]-4′-carboxamide;
(93) trans-3-oxo-N-(1-phenyl-4-pyrazolyl)spiro[7-azaisobenzofuran-1(3H), 1′-cyclohexane]-4′-carboxamide;
(94) trans-3-oxo-N-(1-phenyl-3-pyrrolyl)spiro[7-azaisobenzofuran-1(3H),1′-cyclohexane]-4′-carboxamide;
(95) trans-N-[1-(4-fluorophenyl)-3-pyrazolyl]-3-oxospiro[7-azaisobenzofuran-1(3H),1′-cyclohexane]-4′-carboxamide;
(96) trans-3-oxo-N-(1-phenyl-3-pyrazolyl)spiro[4-azaisobenzofuran-1(3H),1′-cyclohexane]-4′-carboxamide;
(97) trans-3-oxo-N-(1-phenyl-4-pyrazolyl)spiro[4-azaisobenzofuran-1(3H),1′-cyclohexane]-4′-carboxamide;
(98) trans-N-[1-(3-fluorophenyl)-4-pyrazolyl]-3-oxospiro[4-azaisobenzofuran-1(3H),1′-cyclohexane]-4′-carboxamide;
(99) trans-3-oxo-N-(1-phenyl-3-pyrazolyl)spiro[6-azaisobenzofuran-1(3H), 1′-cyclohexane]-4′-carboxamide;
(100) trans-N-[1-(4-fluorophenyl)-3-pyrazolyl]-3-oxospiro[6-azaisobenzofuran-1(3H),1′-cyclohexane]-4′-carboxamide;
(101) trans-N-[1-(2-fluorophenyl)-3-pyrazolyl]-3-oxospiro[6-azaisobenzofuran-1(3H),1′-cyclohexane]-4′-carboxamide;
(102) trans-3-oxo-N-(5-phenyl-1,2,4-thiadiazol-3-yl)spiro[6-azaisobenzofuran-1(3H),1′-cyclohexane]-4′-carboxamide;
(103) trans-3-oxo-N-(5-phenyl-3-isoxazolyl)spiro[6-azaisobenzofuran-1(3H),1′-cyclohexane]-4′-carboxamide;
(104) trans-3-oxo-N-(6-phenyl-3-pyridyl)spiro[6-azaisobenzofuran-1(3H),1′-cyclohexane]-4′-carboxamide;
(105) trans-3-oxo-N-(2-phenyl-3-thiazolyl)spiro[6-azaisobenzofuran-1(3H),1′-cyclohexane]-4′-carboxamide;
(106) trans-3-oxo-N-(2-phenyl-1,2,3-triazol-4-yl)spiro[6-azaisobenzofuran-1(3H),1′-cyclohexane]-4′-carboxamide;
and pharmaceutically acceptable salts and esters thereof.

6. The composition of claim 3 wherein the NPY5 antagonist is selected from the group consisting of a compound of formula II 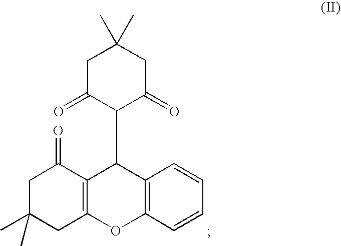
and pharmaceutically acceptable salts and esters thereof.

7. The composition of claim 3 wherein the anti-dyslipidemic agent is a HMG-CoA reductase inhibitor selected from the group consisting of atorvastatin, cerivastatin, itavastatin, fluvastatin, lovastatin, pravastatin, rivastatin, simvastatin, and ZD4522, or a pharmaceutically acceptable salt or ester thereof.

8. The composition of claim 3 wherein the NPY5 antagonist is trans-N-[1-(2-fluorophenyl)-3-pyrazolyl]-3-oxospiro[6-azaisobenzofuran-1(3H),1′-cyclohexane]-4′-carboxamide, or a pharmaceutically acceptable salt thereof, and the anti-dyslipidemic agent is simvastatin, or a pharmaceutically acceptable salt thereof.

9. The composition of claim 3 wherein the anti-dyslipidemic agent is a cholesterol absorption inhibitor selected from the group consisting of beta-sitosterol, ezetimibe, and tiqueside, or a pharmaceutically acceptable salt or ester thereof.

10. The composition of claim 3 wherein the NPY5 antagonist is trans-N-[1-(2-fluorophenyl)-3-pyrazolyl]-3-oxospiro[6-azaisobenzofuran-1(3H),1′-cyclohexane]-4′-carboxamide, or a pharmaceutically acceptable salt thereof, and the anti-dyslipidemic agent is ezetimibe, or a pharmaceutically acceptable salt thereof.

11. The composition of claim 3 wherein the anti-dyslipidemic agent is a composition comprising a cholesterol absorption inhibitor, or a pharmaceutically acceptable salt or ester thereof; and a HMG-CoA reductase inhibitor, or a pharmaceutically acceptable salt or ester thereof.

12. The composition of claim 3 wherein the NPY5 antagonist is trans-N-[1-(2-fluorophenyl)-3-pyrazolyl]-3-oxospiro[6-azaisobenzofuran-1(3H),1′-cyclohexane]-4′-carboxamide, or a pharmaceutically acceptable salt thereof, and the anti-dyslipidemic agent is a composition comprising ezetimibe, or a pharmaceutically acceptable salt thereof; and simvastatin, or a pharmaceutically acceptable salt thereof.

13. A pharmaceutical composition comprising a composition of claim 1 and a pharmaceutically acceptable carrier.

14. The composition of claim 1 wherein
(a) the anti-obesity agent selected from the group consisting of: aminorex; amphechloral; amphetamine; benzphetamine; chlorphentermine; clobenzorex; cloforex; clominorex; clortermine; cyclexedrine; dexfenfluramine; dextroamphetamine; diethylpropion; diphemethoxidine; N-ethylamphetamine; fenbutrazate; fenfluramine; fenisorex; fenproporex; fludorex; fluminorex; furfurylmethylamphetamine; levamfetamine; levophacetoperane; mazindol; mefenorex; metamfepramone; methamphetamine; norpseudoephedrine; pentorex; phendimetrazine; phenmetrazine; phentermine; phenylpropanolamine; and picilorex; and zonisamide; and pharmaceutically acceptable salts and esters thereof; and
(b) the anti-dyslipidemic agent is selected from the group consisting of
(1) bile acid sequesterant,
(2) a HMG-CoA reductase inhibitor,
(3) a HMG-CoA synthase inhibitor,
(4) a cholesterol absorption inhibitor,
(5) an acyl coenzyme A-cholesterol acyl transferase inhibitor,
(6) a cholesteryl ester transfer protein inhibitor,
(7) a squalene synthetase inhibitor,
(8) an anti-oxidant,
(9) a PPAR (x agonist,
(10) a FXR receptor antagonist,
(11) a LXR receptor agonist,
(12) a lipoprotein synthesis inhibitor,
(13) a renin angiotensin system inhibitor,
(14) a microsomal triglyceride transport inhibitor,
(15) a bile acid reabsorption inhibitor,
(16) a PPARδ agonist,
(17) a triglyceride synthesis inhibitor,
(18) a transcription modulator,
(19) a squalene epoxidase inhibitor,
(20) a low density lipoprotein receptor inducer, and
(21) a platelet aggregation inhibitor,
(22) a 5-LO or FLAP inhibitor,
(23) a PPAR δ partial agonist,
(24) niacin or a niacin receptor agonist,
and pharmaceutically acceptable salts and esters thereof;
and a pharmaceutically acceptable carrier.

15. A pharmaceutical composition comprising (1) a composition of claim 1, and (2) one or more compounds selected from the group consisting of:
(a) anti-diabetic agents such as (i) PPARγ agonists such as glitazones (e.g. ciglitazone; darglitazone; englitazone; isaglitazone (MCC-555); pioglitazone; rosiglitazone; troglitazone; CLX-0921; 5-BTZD, and the like), and GW-0207, LG-100641, and LY-300512, and the like; (ii) biguanides such as buformin; metformin; and phenformin, and the like; (iii) protein tyrosine phosphatase-1B (PTP-1B) inhibitors; (iv) sulfonylureas such as acetohexamide; chlorpropamide; diabinese; glibenclamide; glipizide; glyburide; glimepiride; gliclazide; glipentide; gliquidone; glisolamide; tolazamide; and tolbutamide, and the like; (v) meglitinides such as repaglinide, and nateglinide, and the like; (vi) alpha glucoside hydrolase inhibitors such as acarbose; adiposine; camiglibose; emiglitate; miglitol; voglibose; pradimicin-Q; salbostatin; CKD-711; MDL-25,637; MDL-73,945; and MOR 14, and the like; (vii) alpha-amylase inhibitors such as tendamistat, trestatin, and A1-3688, and the like; (viii) insulin secreatagogues such as linogliride; and A-4166, and the like; (ix) fatty acid oxidation inhibitors, such as clomoxir, and etomoxir, and the like; (x) A2 antagonists, such as midaglizole; isaglidole; deriglidole; idazoxan; earoxan; and fluparoxan, and the like; (xi) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH₂), and the like; (xii) non-thiazolidinediones such as JT-501, and farglitazar (GW-2570/GI-262579), and the like; (xiii) PPARα/γ dual agonists such as CLX-0940, GW-1536, GW1929, GW-2433, KRP-297, L-796449, LR-90, MK-0767, SB 219994, and muraglitazar, and the like; (xiv) other insulin sensitizing drugs; (xv) a glucokinase activator; and (xvi) VPAC2 receptor agonists; and
(b) anti-hypertensive agents such as (i) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (ii) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (iii) calcium channel blockers such as amlodipine, aranidipine, azelnidipine, bamidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (iv) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (v) neutral endopeptidase inhibitors such as omapatrilat, cadoxatril and ecadotril, fosidotril, sampatrilat, AVE7688, ER4030, and the like; (vi) endothelin antagonists such as tezosentan, A308165, and YM62899, and the like; (vii) vasodilators such as hydralazine, clonidine, minoxidil, and nicotinyl alcohol, and the like; (viii) angiotensin II receptor antagonists such as candesartan, eprosartan, irbesartan, losartan, pratosartan, tasosartan, telmisartan, valsartan, and EXP-3137, F16828K, and RNH6270, and the like; (viv) ax/, adrenergic blockers as nipradilol, arotinolol and amosulalol, and the like; (x) alpha I blockers, such as terazosin, urapidil, prazosin, bunazosin, trimazosin, doxazosin, naftopidil, indoramin, WHIP 164, and XEN010, and the like; and (xi) alpha 2 agonists such as lofexidine, tiamenidine, moxonidine, rilmenidine and guanobenz, and the like; and (3) a pharmaceutically acceptable carrier.

16. A method of treating or preventing dyslipidemia comprising administration of a therapeutically or prophylactically effective amount of a composition of claim 1 to a subject in need of such treatment.

17. A method of treating or preventing dyslipidemia comprising administration of a therapeutically or prophylactically effective amount of the composition of claim 8 to a subject in need of such treatment.

18. A method of treating or preventing dyslipidemia comprising administration of a therapeutically or prophylactically effective amount of the composition of claim 10 to a subject in need of such treatment.

19. A method of treating or preventing dyslipidemia comprising administration of a therapeutically or prophylactically effective amount of the composition of claim 12 to a subject in need of such treatment.

20. A method of treating or preventing a dyslipidemia-related disorder comprising administration of a therapeutically or prophylactically effective amount of a composition of claim 1 to a subject in need of such treatment.

21. The method according to claim 20 wherein the dyslipidemia-related disorder is selected from the group consisting of hyperlipidemia, hypertriglyceridemia, hypercholesterolemia, low high density lipoprotein cholesterol levels, high low density lipoprotein cholesterol levels, atherosclerosis, coronary artery disease, heart attack, and stroke.

22. The method according to claim 20 wherein the dyslipidemia-related disorder is atherosclerosis.

23. A method of treating or preventing left ventricular hypertrophy comprising administration of a therapeutically or prophylactically effective amount of an anti-obesity agent, or a pharmaceutically acceptable salt thereof, and a therapeutically or prophylactically effective amount of an anti-dyslipidemic agent, or a pharmaceutically acceptable salt thereof, to a subject in need of such treatment.

24. The method of claim 23 wherein the anti-obesity agent is a NPY5 antagonist.

25. A method of treating or preventing left ventricular hypertrophy comprising administration of a therapeutically or prophylactically effective amount of the composition of claim 8 to a subject in need of such treatment.

26. A method of treating or preventing left ventricular hypertrophy comprising administration of a therapeutically or prophylactically effective amount of the composition of claim 10 to a subject in need of such treatment.

27. A method of treating or preventing left ventricular hypertrophy comprising administration of a therapeutically or prophylactically effective amount of the composition of claim 12 to a subject in need of such treatment.

28. A method of treating or preventing obesity comprising administration of a therapeutically or prophylactically effective amount of a composition of claim 1 to a subject in need of such treatment.

29. A method of treating or preventing obesity comprising administration of a therapeutically or prophylactically effective amount of the composition of claim 8 to a subject in need of such treatment.

30. A method of treating or preventing obesity comprising administration of a therapeutically or prophylactically effective amount of the composition of claim 10 to a subject in need of such treatment.

31. A method of treating or preventing obesity comprising administration of a therapeutically or prophylactically effective amount of the composition of claim 12 to a subject in need of such treatment.

32. A method of treating or preventing an obesity-related disorder comprising administration of a therapeutically or prophylactically effective amount of a composition of claim 1 to a subject in need of such treatment.

33. A method of treating or preventing metabolic syndrome comprising administration of a therapeutically or prophylactically effective amount of a composition of claim 1 and a therapeutically or prophylactically effective amount of an anti-hypertensive agent to a subject in need of such treatment.

34. A method of treating or preventing metabolic syndrome comprising administration of a therapeutically or prophylactically effective amount of a composition of claim 1 and a therapeutically or prophylactically effective amount of an anti-diabetic agent to a subject in need of such treatment.

35. A method of treating or preventing metabolic syndrome comprising administration of a therapeutically or prophylactically effective amount of the composition of claim 8 to a subject in need of such treatment.

36. A method of treating or preventing metabolic syndrome comprising administration of a therapeutically or prophylactically effective amount of the composition of claim 10 to a subject in need of such treatment.

37. A method of treating or preventing metabolic syndrome comprising administration of a therapeutically or prophylactically effective amount of the composition of claim 12 to a subject in need of such treatment.

38. A method of treating or preventing metabolic syndrome comprising administration of a therapeutically or prophylactically effective amount of a composition of claim 1, a therapeutically or prophylactically effective amount of an anti-diabetic agent, and a therapeutically effective or prophylactically amount of an anti-hypertensive agent to a subject in need of such treatment.

39. A method of treating, controlling, or preventing metabolic syndrome, said method comprising the administration of an effective amount of a composition of claim 1, and an effective amount of one or more other compounds selected from the group consisting of:
(a) anti-diabetic agents such as (i) PPARγ agonists such as glitazones (e.g. ciglitazone; darglitazone; englitazone; isaglitazone (MCC-555); pioglitazone; rosiglitazone; troglitazone; CLX-0921; 5-BTZD, and the like), and GW-0207, LG-100641, and LY-300512, and the like; (ii) biguanides such as buformin; metformin; and phenformin, and the like; (iii) protein tyrosine phosphatase-1B (PTP-1B) inhibitors; (iv) sulfonylureas such as acetohexamide; chlorpropamide; diabinese; glibenclamide; glipizide; glyburide; glimepiride; gliclazide; glipentide; gliquidone; glisolamide; tolazamide; and tolbutamide, and the like; (v) meglitinides such as repaglinide, and nateglinide, and the like; (vi) alpha glucoside hydrolase inhibitors such as acarbose; adiposine; camiglibose; emiglitate; miglitol; voglibose; pradimicin-Q; salbostatin; CKD-711; MDL-25,637; MDL-73,945; and MOR 14, and the like; (vii) alpha-amylase inhibitors such as tendamistat, trestatin, and A1-3688, and the like; (viii) insulin secreatagogues such as linogliride; and A-4166, and the like; (ix) fatty acid oxidation inhibitors, such as clomoxir, and etomoxir, and the like; (x) A2 antagonists, such as midaglizole; isaglidole; deriglidole; idazoxan; earoxan; and fluparoxan, and the like; (xi) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH₂), and the like; (xii) non-thiazolidinediones such as JT-501, and farglitazar (GW-2570/GI-262579), and the like; (xiii) PPARα/γ dual agonists such as CLX-0940, GW-1536, GW1929, GW-2433, KRP-297, L-796449, LR-90, MK-0767, and SB 219994, and muraglitizar, and the like; (xiv) other insulin sensitizing drugs; (xv) a glucokinase activator; and (xvi) VPAC2 receptor agonists; and
(b) anti-hypertensive agents such as (i) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (ii) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (iii) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (iv) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (v) neutral endopeptidase inhibitors such as omapatrilat, cadoxatril and ecadotril, fosidotril, sampatrilat, AVE7688, ER4030, and the like; (vi) endothelin antagonists such as tezosentan, A308165, and YM62899, and the like; (vii) vasodilators such as hydralazine, clonidine, minoxidil, and nicotinyl alcohol, and the like; (viii) angiotensin II receptor antagonists such as candesartan, eprosartan, irbesartan, losartan, pratosartan, tasosartan, telmisartan, valsartan, and EXP-3137, F16828K, and RNH6270, and the like; (viv) α/β adrenergic blockers as nipradilol, arotinolol and amosulalol, and the like; (x) alpha 1 blockers, such as terazosin, urapidil, prazosin, bunazosin, trimazosin, doxazosin, naftopidil, indoramin, WHIP 164, and XEN010, and the like; and (xi) alpha 2 agonists such as lofexidine, tiamenidine, moxonidine, rilmenidine and guanobenz, and the like.

40-51. (canceled)
